(19) United States
(12) Patent Application Publication
(10) Pub. No.: US 2023/0233458 A1
(43) Pub. Date: Jul. 27, 2023

(54) Gastroretentive structured dosage form

(71) Applicant: Aron H. Blaesi, Cambridge, MA (US)

(72) Inventors: Aron H. Blaesi, Cambridge, MA (US); Nannaji Saka, Cambridge, MA (US)

(73) Assignee: Aron H. Blaesi, Cambridge, MA (US)

(21) Appl. No.: 18/124,381

(22) Filed: Mar. 21, 2023

Related U.S. Application Data
(63) Continuation of application No. PCT/US2021/053027, filed on Sep. 30, 2021.
(60) Provisional application No. 63/085,893, filed on Sep. 30, 2020.
(60) Provisional application No. 63/158,870, filed on Mar. 9, 2021.
(60) Provisional application No. 63/229,016, filed on Aug. 3, 2021.
(60) Provisional application No. 63/247,291, filed on Sep. 22, 2021.

(51) Int. Cl. A61K 9/00; A61K 31/192; A61K 51/12; A61K 9/20

(52) U.S. Cl. CPC A61K 9/0065; A61K 31/192; A61K 51/1258; A61K 9/2054; A61K 9/2027

(57) ABSTRACT

Many drug therapies could be greatly improved by dosage forms that reside in the stomach for prolonged time and release the drug slowly. In this specification, therefore, a gastroretentive structured dosage form is disclosed. The dosage form comprises a solid core having at least a fluid-absorptive first excipient. The dosage form further comprises a semi-permeable surface layer substantially encapsulating said solid core. The surface layer comprises at least a mechanically strengthening second excipient. Upon ingestion, the surface layer-supported solid core expands with physiological fluid absorption and remains in the stomach for prolonged time.

### CROSS-REFERENCE TO RELATED INVENTIONS

This application is a continuation of, and incorporates herein by reference in its entirety, the International Application No. PCT/US21/053027 filed on Sep. 30, 2021 and titled “Gastroretentive structured dosage form”, which claims priority to and the benefit of, the U.S. Provisional Application No. 63/085,893 filed on Sep. 30, 2020, the U.S. Provisional Application No. 63/158,870 filed on Mar. 9, 2021, the U.S. Provisional Application No. 63/229,016 filed on Aug. 3, 2021, and the U.S. Provisional Application No. 63/247,291 filed on Sep. 22, 2021. All foregoing applications are hereby incorporated by reference in their entirety.

This application is related to and incorporates herein by reference in their entirety, the U.S. application Ser. No. 15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, the U.S. application Ser. No. 16/860,911 filed on Apr. 28, 2020 and titled “Expandable structured dosage form for immediate drug delivery”, the U.S. application Ser. No. 16/916,208 filed on Jun. 30, 2020 and titled “Dosage form comprising structural framework of two-dimensional elements”, the U.S. application Ser. No. 17/237,034 filed on Apr. 21, 2021 and titled “Method for 3D-micro-patterning”, the U.S. application Ser. No. 17/327,721 filed on May 23, 2021 and titled “Expandable multi-excipient dosage form”, the International Application No. PCT/US19/19004 filed on Feb. 21, 2019 and titled “Expanding structured dosage form”, the International Application No. PCT/US19/52030 filed on Sep. 19, 2019 and titled “Dosage form comprising structured solid-solution framework of sparingly-soluble drug and method for manufacture thereof”, the International Application No. PCT/US21/22857 filed on Mar. 17, 2021 and titled “Expandable, multi-excipient structured dosage form for prolonged drug release”, and the International Application No. PCT/US21/22860 filed on Mar. 17, 2021 and titled “Method and apparatus for 3D-micro-patterning”.

### BACKGROUND OF THE INVENTION

For decades, the development of gastroretentive dosage forms has been the subject of intense research. Such dosage forms enable the release of drug into the stomach for prolonged time, and hence better control of drug absorption time and drug concentration in blood. This in turn enables improved efficacy, safety, and convenience of many prevailing drug therapies. For a non-limiting overview of advantages of gastroretentive dosage forms, see e.g., S. S. Davis et al., The effect of density on the gastric emptying of single- and multiple-unit dosage forms, Pharm. Res. 3 (1986) 208-213; S. S. Davis, Formulation strategies for absorption windows, Drug discovery today 10 (2005) 249-257; A. Streubel, J. Siepmann, R. Bodmeier, Gastroretentive drug delivery systems, Expert Opin. Drug Deliv. 3 (2006) 217-233; and R. Langer, G. Traverso, Special delivery for the gut, Nature 519 (2015) S19.

The concepts mostly examined for gastric retention are the mucoadhesive, floating, and expandable dosage forms. The mucoadhesive forms are designed to adhere to the stomach walls, while the floating forms float over the gastric contents in the upper stomach. Both concepts, however, have not shown any significant increase in gastric residence time.

The expandable dosage forms are more promising. They are smaller than the diameter of the esophagus to facilitate ingestion. But in the stomach they expand to a size greater than the diameter of the pylorus, thus precluding their immediate passage into the small intestine.

The most common types of expandable dosage forms are the swelling and the unfolding devices. The swelling dosage forms generally transition to a low-viscosity mass upon water absorption, which deforms and disintegrates fast, and thus the gastric residence time is limited.

The unfolding devices, by contrast, can be strong. However, because the unfolded device is slender, its mechanical components must be rigid enough to prevent premature deformation and disintegration. Such rigid components may injure the gastric mucosa. For further details on prior expandable gastroretentive dosage forms and their limitations, see, e.g., E. A. Klausner et al., Expandable gastroretentive dosage forms, J. Control. Release 90 (2003) 143-162; K. C. Waterman, A critical review of gastric retentive controlled drug delivery, Pharm. Dev. and Tech. 12 (2007) 1-10; A. M. Bellinger, et al., Oral, ultra-long-lasting drug delivery: Application toward malaria elimination goals, Sci. Trans. Med. 8, 365ra157 (2016) 1-12.

To overcome the limitations of the prior art, therefore, in the International Application No. PCT/US2021/022857 and in the publications Mater. Sci. Eng. C 120 (2021) 110144 and Int. J. Pharm., 120396, in press, the present inventors (Blaesi and Saka) have introduced expandable fibrous dosage forms. As shown schematically in the non-limiting FIG. 1, a promising dosage form consisted of a sparingly-soluble drug; water-absorbing, high-molecular-weight excipient; and fiber-strengthening, enteric excipient. Upon immersion in a dissolution fluid, the dosage form expanded rapidly and formed a semi-solid mass. Depending on the volume fraction of fibers, 80 percent of the drug was released in about 2-40 hours.

In the stomach, however, the loads applied on the expanded semi-solid dosage form are expected to be greater than those in a stirred dissolution vessel. For further information on stomach physiology, see, e.g., H. Minami, R. W. McCallum, The physiology and pathophysiology of gastric emptying in humans. Gastroenterology 86 (1984) 1592-1610.

To assure that the expanded dosage form is retained in the stomach for the desired time, in the present disclosure new dosage form microstructures and formulations are presented.

### SUMMARY OF THE INVENTION

In one aspect, the invention herein comprises a pharmaceutical dosage form comprising a drug-containing solid having a fluid-absorptive solid core and a mechanically strengthening, semi-permeable surface layer; said fluid-absorptive solid core comprising at least a fluid-absorptive first excipient; said fluid-absorptive solid core further substantially encapsulated by said mechanically strengthening, semi-permeable surface layer, said semi-permeable surface layer comprising at least a mechanically strengthening second excipient; whereby upon exposure of the dosage form to a physiological fluid, the surface layer-encapsulated solid core expands with fluid absorption.

In some embodiments, upon exposure of the dosage form to a physiological fluid, the surface layer-encapsulated solid core expands primarily with fluid absorption.

In some embodiments, the surface layer-encapsulated solid core transitions to a viscous or semi-solid mass as it expands with fluid absorption.

In some embodiments, upon exposure of the dosage form to a physiological fluid, the mechanically strengthening, semi-permeable surface layer forms a semi-permeable, viscoelastic membrane.

In some embodiments, upon exposure of the dosage form to a physiological fluid, said mechanically strengthening, semi-permeable surface layer is substantially permeable to said physiological fluid.

In some embodiments, upon exposure of the dosage form to a physiological fluid, said mechanically strengthening, semi-permeable surface layer is substantially impermeable to at least one fluid-absorptive first excipient.

In some embodiments, upon exposure of the dosage form to a physiological fluid, the mechanically strenghtening, semi-permeable surface layer expands due to an internal pressure in the core, said internal pressure generated by osmotic flow of fluid into said core.

In some embodiments, upon exposure of the dosage form to a physiological fluid, the drug-containing solid forms an expanded, viscoelastic composite mass.

In another aspect, the invention herein comprises a pharmaceutical dosage form comprising a drug-containing solid having a fluid-absorptive solid core and a mechanically strengthening, semi-permeable surface layer; said fluid-absorptive solid core comprising at least a fluid-absorptive first excipient; said fluid-absorptive solid core further substantially encapsulated by said mechanically strengthening, semi-permeable surface layer, said semi-permeable surface layer comprising at least a mechanically strengthening second excipient; whereby upon exposure of the dosage form to a physiological fluid, the surface layer-encapsulated solid core expands primarily with fluid absorption, thereby transitioning to a viscous or semi-solid mass; and the mechanically strengthening, semi-permeable surface layer forms a semi-permeable, viscoelastic membrane; wherein said semi-permeable, viscoelastic membrane expands due to an internal pressure in the core generated by osmotic flow of fluid into said core; and the drug-containing solid forms an expanded, viscoelastic composite mass.

In some embodiments, the fluid-absorptive solid core has at least one dimension greater than 6 mm (e.g., greater than 7 mm, or greater than 8 mm).

In some embodiments, upon exposure of the dosage form to a physiological fluid, the drug-containing solid forms an expanded, surface layer-supported viscoelastic composite mass having a length between 1.2 and 5 times (e.g., between 1.3 and 4 times, or between 1.4 and 4 times) its length prior to exposure to said physiological fluid.

In some embodiments, upon exposure of the dosage form to a physiological fluid for no more than 10 hours (e.g., for no more than 8 hours, or for no more than 6 hours, or for no more than 5 hours), the drug-containing solid forms an expanded, surface layer-supported viscoelastic composite mass having a length between 1.3 and 5 times its length prior to exposure to said physiological fluid.

In another aspect, the invention herein comprises a pharmaceutical dosage form comprising a drug-containing solid having a fluid-absorptive solid core and a mechanically strengthening, semi-permeable surface layer, said fluid-absorptive solid core having at least one dimension greater than 6 mm; said fluid-absorptive solid core comprising at least a fluid-absorptive first excipient; said fluid-absorptive solid core further substantially encapsulated by said mechanically strengthening, semi-permeable surface layer, said semi-permeable surface layer comprising at least a mechanically strengthening second excipient; whereby upon exposure of the dosage form to a physiological fluid, the surface layer-encapsulated solid core expands primarily with fluid absorption, thereby transitioning to a viscous or semi-solid mass; and the mechanically strengthening, semi-permeable surface layer forms a semi-permeable, viscoelastic membrane; wherein said semi-permeable, semi-solid membrane expands due to an internal pressure in the core generated by osmotic flow of fluid into said core, so that within no more than 10 hours of exposure to said physiological fluid the drug-containing solid forms an expanded, viscoelastic composite mass having a length between 1.3 and 5 times its length priorto exposure to said physiological fluid.

In some embodiments, the solubility of a physiological fluid in one or more fluid-absorptive excipients is greater than 600 mg/ml (e.g., greater than 700 mg/ml, or greater than 800 mg/ml).

In some embodiments, upon exposure to a physiological fluid under physiological conditions, the diffusivity of said physiological fluid through a fluid-absorptive core is greater than 0.2×10⁻¹² m²/s (e.g., greater than 0.5×10⁻² m²/s, or greater than 10-12 m²/s).

In some embodiments, at least one fluid-absorptive excipient comprises hydroxypropyl methylcellulose.

In some embodiments, at least one fluid-absorptive first excipient comprises hydroxypropyl methylcellulose with an average molecular weight greater than 30 kg/mol.

In some embodiments, at least one fluid-absorptive first excipient comprises hydroxypropyl methylcellulose with an average molecular weight greater than 30 kg/mol, and wherein the volume or weight fraction of hydroxypropyl methylcellulose with average molecular weight greater than 30 kg/mol in the fluid-absorptive solid core is greater than 0.1.

In some embodiments, at least one fluid-absorptive excipient is selected from the group comprising hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), polyacrylic acid, polyethylene oxide, or vinylpyrrolidone-vinyl acetate copolymer.

In some embodiments, average molecular weight of one or more fluid-absorptive excipients is in the range of 30 kg/mol to 100,000 kg/mol (e.g., in the range of 40 kg/mol to 50,000 kg/mol, or in the range of 50 kg/mol to 50,000 kg/mol).

In some embodiments, volume or weight fraction of one or more fluid-absorptive excipients in the fluid-absorptive solid core is greater than 0.1 (e.g., greater than 0.15, or greater than 0.2).

In some embodiments, the solubility of a mechanically strengthening second excipient is no greater than 0.5 mg/ml (e.g., no greater than 0.2 mg/ml, or no greater than 0.1 mg/ml, or no greater than 0.05 mg/ml) in a relevant physiological fluid (e.g., gastric fluid) under physiological conditions.

In some embodiments, the solubility of a relevant physiological fluid in at least one mechanically strengthening second excipient is no greater than 750 mg/ml (e.g., no greater than 650 mg/ml, or no greater than 550 mg/ml) under physiological conditions.

In some embodiments, at least a mechanically strengthening second excipient (or the strength-enhancing excipient in its totality, or a mechanically strengthening, semi-permeable surface layer) comprises a strain at fracture greater than 0.4 (e.g., greater than 0.5, or greater than 0.6, or greater than 0.8, or greater than 1) after soaking with a physiological fluid under physiological conditions.

In some embodiments, at least one mechanically strengthening second excipient (or the strength-enhancing excipient in its totality, or a mechanically strengthening, semi-permeable surface layer) comprises an elastic modulus in the range of 0.1 MPa-100 MPa (e.g., 0.2 MPa-50 MPa, or 0.5 MPa-20 MPa) after soaking with a physiological fluid under physiological conditions.

In some embodiments, at least one mechanically strengthening second excipient (or the strength-enhancing excipient in its totality, or a mechanically strengthening, semi-permeable surface layer) comprises a tensile strength in the range of 0.05 MPa-100 MPa (e.g., 0.1 MPa-50 MPa, or 0.2 MPa-20 MPa) after soaking with a physiological fluid under physiological conditions.

In some embodiments, elongational viscosity of mechanically strengthening, semi-permeable surface layer is in the range of 5×10⁵ Pa·s-1×10¹¹ Pa·s (e.g., 1×10⁶ Pa·s-5×10¹⁰ Pa·s, 2×10⁶ Pa·s-1×10¹⁰ Pa·s) after soaking with a physiological fluid under physiological conditions.

In some embodiments, at least one mechanically strengthening second excipient comprises an enteric polymer.

In some embodiments, at least one mechanically strenghtening second excipient comprises an enteric polymer, said enteric polymer having a solubility at least 10 times (e.g., at least 100 times) greater in basic solution having a pH value greater than 7 (e.g., greater than 8) than in acidic solution having a pH value no greater than 5 (e.g., no greater than 4).

In some embodiments, at least one mechanically strengthening second excipient comprises methacrylic acid-ethyl acrylate copolymer.

In some embodiments, at least one mechanically strengthening second excipient comprises polyvinyl acetate.

In some embodiments, at least one mechanically strengthening second excipient is selected from the group comprising methacrylic acid-ethyl acrylate copolymer, methacrylic acic-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose.

In some embodiments, said fluid-absorptive solid core comprises at least a drug.

In some embodiments, said fluid-absorptive solid core comprises a mixture of drug and at least a fluid-absorptive first excipient.

In some embodiments, upon exposure to a physiological fluid, the drug-containing solid or semi-solid releases drug overtime (e.g., over a time greater than 30 minutes, or over a time greater than 1 hour, or over a time greater than 2 hours).

In some embodiments, said fluid-absorptive solid core comprises at least a mechanically strengthening third excipient.

In some embodiments, said fluid-absorptive solid core comprises a mixture of drug, at least a fluid-absorptive first excipient, and at least a mechanically strengthening third excipient.

In some embodiments, at least one mechanically strengthening third excipient comprises methacrylic acid-ethyl acrylate copolymer.

In some embodiments, said fluid-absorptive solid core comprises a three-dimensional structural framework of structural elements.

In some embodiments, the thickness of one or more structural elements is precisely controlled.

In some embodiments, average thickness of one or more structural elements is in the range between 5 μm and 2.5 mm (e.g., between 10 μm and 2.5 mm, or between 10 μm and 2 mm).

In some embodiments, one or more structural elements are repeatably arranged.

In some embodiments, one or more elements comprise segments spaced apart from adjoining segments by element-free spacings, thereby defining one or more element-free spaces in the drug-containing solid.

In some embodiments, average element-free spacing across one or more element-free spaces is in the range between 10 μm and 4 mm (e.g., between 10 μm and 3 mm, or between 10 μm and 2 mm).

In some embodiments, the spacing between elements or segments across the three-dimensional structural framework is precisely controlled.

In some embodiments, one or more surface layer-encapsulated elements comprise surface layer-encapsulated segments spaced apart from adjoining surface layer-encapsulated segments by free spacings, thereby defining one or more free spaces in the drug-containing solid.

In some embodiments, average free spacing across one or more free spaces is in the range between 5 μm and 3 mm (e.g., between 5 μm and 2 mm, or between 5 μm and 1.5 mm).

In some embodiments, at least one free space is filled with matter removable by a physiological fluid under physiological conditions.

In some embodiments, at least one free space is filled with a matter comprising a gas.

In some embodiments, a three dimensional structural framework of elements comprises an outer surface and an outer volume, said outer volume defined by the volume enclosed by said outer surface, and wherein the volume fraction of fluid-absorptive structural elements within said outer volume is in the range between 0.05 and 0.95 (e.g., between 0.1 and 0.95, or between 0.15 and 0.95, or between 0.2 and 0.95).

In some embodiments, a three dimensional structural framework of elements comprises an outer surface and an outer volume, said outer volume defined by the volume enclosed by said outer surface, and wherein the volume fraction of mechanically strengthening, semi-permeable surface layer within said outer volume is in the range between 0.005 and 0.5 (e.g., between 0.01 and 0.4, or between 0.015 and 0.3).

In some embodiments, a thickness of a mechanically strengthening, semi-permeable surface layer is greater than 1 μm (e.g., greater than 2 μm, or greater than 5 μm).

In some embodiments, a three dimensional structural framework comprises a single continuous structure.

In some embodiments, an element or framework comprises a plurality of segments having substantially the same weight fraction of physiological fluid-absorptive excipient distributed within the segments.

In some embodiments, a mechanically strengthening, semi-permeable surface layer forms a substantially connected structure through the three dimensional structural framework.

In some embodiments, one or more structural elements comprise one or more fibers.

In some embodiments, said fluid-absorptive solid core comprises a three-dimensional structural network of criss-crossed stacked layers of fibers.

In another aspect, the invention herein comprises a pharmaceutical dosage form comprising a drug-containing solid having a fluid-absorptive solid core and a semi-permeable surface layer; said fluid-absorptive solid core comprising a three-dimensional structural framework of one or more structural elements; said elements comprising a mixture of drug and at least a first excipient, wherein said first excipient includes at least a fluid-absorptive polymeric constituent; said semi-permeable surface layer substantially encapsulating said elements; said semi-permeable surface layer further comprising at least a second excipient, said second excipient including at least a mechanically strengthening polymeric constituent; whereby upon exposure of the dosage form to physiological fluid, the surface layer-supported solid core expands with fluid absorption, thereby forming a viscoelastic composite mass.

In yet another aspect, the invention herein comprises a pharmaceutical dosage form comprising a drug-containing solid having a fluid-absorptive solid core and a semi-permeable surface layer; said fluid-absorptive solid core comprising a three-dimensional structural network of criss-crossed stacked layers of fibers; said fibers comprising a mixture of drug and at least a first excipient, wherein said first excipient includes at least a fluid-absorptive polymeric constituent; said semi-permeable surface layer substantially encapsulating said fibers; said semi-permeable surface layer further comprising at least a second excipient, said second excipient including at least a mechanically strengthening polymeric constituent; whereby upon exposure of the dosage form to physiological fluid, the surface layer-supported solid core expands with fluid absorption, thereby forming a viscoelastic composite mass.

In a further aspect, the invention herein comprises a pharmaceutical dosage form comprising: a drug-containing solid having a fluid-absorptive solid core and a semi-permeable surface layer; said fluid-absorptive solid core comprising a three-dimensional structural network of criss-crossed stacked layers of fibers, said fibers having an average fiber thickness in the range of 5 μm to 2 mm; said fibers further comprising fiber segments spaced apart from adjoining segments by fiber-free spacings, thereby defining one or more fiber-free spaces in the drug-containing solid; said fibers further comprising a mixture of drug and at least a first excipient, wherein said first excipient includes at least a fluid-absorptive polymeric constituent; said fibers further substantially encapsulated by said mechanically strengthening semi-permeable surface layer, said semi-permeable surface layer comprising at least a mechanically strengthening second excipient; whereby upon exposure of the dosage form to physiological fluid, the surface layer-supported solid core expands with fluid absorption, thereby forming a viscoelastic composite mass.

In a further aspect, the invention herein comprises a pharmaceutical dosage form comprising a drug-containing solid having a fluid-absorptive solid core and a semi-permeable surface layer; said fluid-absorptive solid core comprising a three-dimensional structural network of criss-crossed stacked layers of fibers, said fibers having an average fiber thickness in the range of 5 μm to 2 mm; said fibers further comprising fiber segments spaced apart from adjoining segments by fiber-free spacings, thereby defining one or more fiber-free spaces in the drug-containing solid; said fibers further comprising a mixture of drug and at least a first excipient, wherein said first excipient includes at least a fluid-absorptive polymeric constituent; said fibers further substantially encapsulated by said mechanically strengthening semi-permeable surface layer, said semi-permeable surface layer comprising at least a mechanically strengthening second excipient; said surface layer-encapsulated fibers comprising surface layer-encapsulated segments spaced apart from adjoining surface layer-encapsulated segments by free spacings, thereby defining one or more free spaces in the drug-containing solid; whereby upon exposure of the dosage form to physiological fluid, the surface layer-supported solid core expands with fluid absorption, thereby forming a viscoelastic composite mass.

In some embodiments, an expanded, surface layer-supported viscoelastic composite mass comprises an elastic modulus in the range of 0.005 MPa-15 MPa (e.g., 0.01 MPa-10 MPa, or 0.01 MPa-5 MPa).

In some embodiments, an expanded, surface layer-supported viscoelastic composite mass comprises a tensile strength in the range between 0.002 MPa and 15 MPa (e.g., between 0.005 MPa and 10 MPa, or between 0.0075 MPa and 5 MPa).

In some embodiments, upon prolonged exposure to a physiological fluid, said expanded framework or viscoelastic composite mass maintains its length between 1.3 and 5 times the initial length for prolonged time (e.g., for a time longer than 20 hours, or for a time longer than 30 hours, or for a time longer than 40 hours).

In some embodiments, upon prolonged exposure to a physiological fluid, said expanded framework or viscoelastic composite mass maintains a tensile strength greater than 0.005 MPa (e.g., greater than 0.0075 MPa) over a time greater than 15 hours (e.g., over a time greater than 25 hours, or over a time greater than 35 hours).

In some embodiments, upon immersion in a physiological fluid, the drug-containing solid transitions to a viscoelastic composite mass comprising a length in the range between 1.3 and 3.5 times its length prior to exposure to said physiological fluid within no more than 500 minutes (e.g., no more than 300 minutes) of immersion in said physiological fluid.

In some embodiments, upon ingestion by a human or animal subject, said dosage form is gastroretentive.

In yet another aspect, the invention herein comprises a pharmaceutical dosage form comprising a drug-containing solid having a fluid-absorptive solid core and a mechanically strengthening, semi-permeable surface layer; said fluid-absorptive solid core comprising a three-dimensional structural framework of structural elements; said structural elements comprising at least a fluid-absorptive first excipient; said structural elements further substantially encapsulated by said mechanically strengthening, semi-permeable surface layer, said semi-permeable surface layer comprising at least a mechanically strengthening second excipient; whereby upon exposure of the dosage form to physiological fluid, the surface layer-supported structural framework expands with fluid absorption.

In a further aspect, the invention herein comprises a pharmaceutical dosage form comprising a drug-containing solid having a fluid-absorptive solid core and a mechanically strengthening, fluid-permeable surface layer; said fluid-absorptive solid core comprising a three-dimensional structural framework of structural elements; said structural elements comprising at least a fluid-absorptive first excipient; said structural elements further substantially encapsulated by said mechanically strengthening, fluid-permeable surface layer, said fluid-permeable surface layer comprising at least a mechanically strengthening second excipient; whereby upon exposure of the dosage form to physiological fluid, the surface layer-supported structural framework expands with fluid absorption.

Embodiments or parts of embodiments described with respect to one aspect of the invention can be applied with respect to another aspect. By way of example but not by way of limitation, certain embodiments of the claims described with respect to the first aspect can include features of the claims described with respect to the second or third aspect, etc. and vice versa.

This invention may be better understood by reference to the accompanying drawings, attention being called to the fact that the drawings are primarily for illustration, and should not be regarded as limiting.

### BRIEF DESCRIPTION OF THE DRAWINGS

The objects, embodiments, features, and advantages of the present invention are more fully understood when considered in conjunction with the following accompanying drawings:

FIG. 1 presents a non-limiting schematic of an expandable fibrous dosage form as previously disclosed;

FIG. 2 shows a non-limiting example of a pharmaceutical dosage form as disclosed herein, and its expansion upon immersion in a dissolution fluid (throughout this disclosure the following symbols represent the following: t: time, to: immersion time, t₁: a specific time after immersion);

FIG. 3 presents another non-limiting example of a pharmaceutical dosage form according to the invention herein, and its expansion upon immersion in a dissolution fluid;

FIG. 4 presents a further non-limiting example of a pharmaceutical dosage form according to the invention herein, and its expansion upon immersion in a dissolution fluid;

FIG. 5 schematically illustrates a non-limiting course of a disclosed dosage form after ingestion by a human or animal subject;

FIG. 6 presents another non-limiting example of a pharmaceutical dosage form according to the invention herein, and its expansion upon immersion in a dissolution fluid;

FIG. 7 presents a non-limiting example of a fiber in diffusion-limited expansion: (a) fiber immediately after immersion in a physiological or dissolution fluid, and (b) fiber at time t after immersion (the symbols represent the following: c_{w}: water concentration in fiber, c_{b}: boundary concentration of water in fiber, r: radial coordinate, R₀: initial fiber radius, R_{f}: radius of expanding or expanded fiber);

FIG. 7 presents a non-limiting schematic of a thinly-coated fiber in diffusion-limited expansion: (a) fiber immediately after immersion in a physiological or dissolution fluid, and (b) fiber at time t after immersion;

FIG. 8 presents a non-limiting schematic of a coated fiber in strain rate-limited expansion: (a) fiber immediately after immersion in a physiological or dissolution fluid, and (b) fiber at time t after immersion;

FIG. 9 is a non-limiting schematic to visualize a non-limiting model for deriving the gastric residence time of a dosage form in static fatigue. P: load per unit length; Pₘₐₓ: maximum load intensity due to contracting stomach walls; σ: tensile stress; σₘₐₓ: maximum tensile stress due to contracting stomach walls; R_{df}: radius of expanded dosage form; tᵣ: gastric residence time;

FIG. 10 is a non-limiting schematic of a dosage form exposed to cyclic loading. P: load per unit length; Pₘₐₓ: maximum load intensity due to contracting stomach walls; σ: tensile stress; σₘₐₓ: maximum tensile stress due to contracting stomach walls; a: semi-width of contact; R_{df}: radius of expanded dosage form; tₚᵤₗₛₑ: period of contraction pulse;

FIG. 11 presents a non-limiting dosage form core according to the invention herein along with its microstructure;

FIG. 12 presents a non-limiting fibrous microstructure of a dosage form herein, and a histogram of the length of fiber segments between adjacent contacts;

FIG. 13 shows another non-limiting fibrous microstructure herein, and a histogram of the angle between contacting fibers;

FIG. 14 presents a non-limiting dosage form according to the invention herein along with its microstructure;

FIG. 15 presents non-limiting examples of solid cores substantially encapsulated by mechanically strengthening, semi-permeable surface layers according to the invention herein;

FIG. 16 presents scanning electron micrographs of dosage forms dip-coated with mechanically strengthening, enteric excipient: (a) low-magnification image of top and (b) front views of the microstructure, and (c) high-magnification image of the cross-section of a coated fiber;

FIG. 17 presents top-view images of non-limiting experimental dosage forms after immersion in a dissolution fluid: (a) sugar and (b) enteric-coated dosage form;

FIG. 18 plots the normalized radial expansion of the dosage forms, ΔR_{df}/R_{df,0}, versus time, t;

FIG. 19 presents images of non-limiting experimental dosage forms at different times during diametral compression: (a) dosage form with sugar-coated and (b) enteric-excipient-coated fibers;

FIG. 20 shows images of a non-limiting experimental, expanded dosage form with enteric-excipient-coated fibers: (a) before and (b) after diametral compression. The compression-tested coated dosage form had visible cracks along the axis of symmetry;

FIG. 21 presents results of diametral compression tests of non-limiting experimental dosage forms: (a) load intensity, P, versus displacement, δ, of dosage forms with enteric-excipient-coated and sugar-coated fibers, and (b) dP/dδ versus δ in diametral compression. The inset of FIG. 21a shows a schematic of the loads applied on a homogeneous, isotropic, linear elastic cylinder compressed by diametrically opposed flat platen. P is the load intensity or force per unit thickness of the cylinder. R_{df} is the radius of the cylinder (or expanded dosage form). The small arrows represent the Hertzian contact pressure distributed over the contact width 2a;

FIG. 22 illustrates the position and shape of a non-limiting experimental dosage form with sugar-coated fibers after administration to a fasted dog. Dry food was given 4-6 hours after administration; it is visible in the bottom row images. The images were obtained by biplanar fluoroscopy. They show the abdomen in lateral projection (cranial left, caudal right);

FIG. 23 illustrates the position and shape of a non-limiting experimental dosage form with enteric-excipient-coated fibers after administration to a fasted dog. Dry food was given 4-6 hours and 30 hours after administration. The images were obtained by biplanar fluoroscopy. They show the abdomen in lateral projection (cranial left, caudal right);

FIG. 24 shows the normalized expansion of the radius of non-limiting experimental dosage forms in vivo and compares it with in vitro data: (a) normalized radial expansion, ΔR_{df}/R_{df,0}, versus time, t, after administration of the dosage forms to the dogs, and (b) in vivo/in vitro comparison of ΔR_{df}/R_{df,0}, versus t;

FIG. 25 presents fluoroscopic image sequences of non-limiting experimental dosage forms during contraction pulsing by the stomach walls: (a) dosage form with sugar-coated fibers 2 hours after administration, and (b) dosage form with enteric excipient coated fibers 7 hours after administration;

FIG. 26 presents results of sorption of dissolution fluid by non-limiting films of strengthening, enteric excipient: (a) weight fraction of water versus time after immersion, and (b) M_{w}(t)/IM_{w,∞} versus t^{1/2}/h₀;

FIG. 27 presents results of nominal tensile stress, σ, versus engineering strain, ε, in thin, acidic water-soaked tensile specimen films of Eudragit L100-55. The stress was derived as: σ=F/Wh where F is the force applied by the grips, W the width of the thin section of the specimen film, and h its thickness. The engineering strain, ε=ΔL/L₀ where ΔL is the distance travelled by the grips and L₀ the initial distance between grips;

FIG. 28 presents scanning electron micrographs of a non-limiting experimental fibrous dosage form with uncoated fibers: (a) top and (b) front views of the microstructure;

FIG. 29 depicts scanning electron micrographs of non-limiting experimental, coated fibrous dosage forms: (a) c_{coat}=60 mg/ml (φ_{c,n}=0.025), (b) c_{coat}=100 mg/ml ((φ_{c,n}=0.041), and (c) c_{coat}=166 mg/ml (φ_{c,n}=0.068). c_{coat}: concentration of polymer in the dip-coating solution; φ_{c,n} nominal volume fraction of coating in dosage form by Eq. (37), Table 5;

FIG. 30 shows top-view images of non-limiting experimental dosage forms after immersion in a dissolution fluid: (a) (φ_{c,n}=0.025, (b) (φ_{c,n}=0.041, and (c) (φ_{c,n}=0.068. (φ_{c,n} is the nominal volume fraction of coating in the solid dosage forms;

FIG. 31 presents the normalized radial expansion of non-limiting experimental dosage forms, ΔR_{df}/R_{df,0}, versus time, t: (a) measured data over the entire range, and (b) data truncated to the region t=[0, tₑₓₚ] where tₑₓₚ is the expansion time. The fit equations are A: ΔR_{df}/R_{df,0}=0.25 t, B: ΔR_{df}/R_{df,0}=0.15 t, and C: ΔR_{df}/R_{df,0}=0. It;

FIG. 32 presents load intensity, P, versus displacement, 8, in diametral compression of non-limiting experimental, expanded dosage forms. The load intensity, P, is the force per unit thickness of the expanded dosage form. The experiments were conducted at the time t=tₑₓₚ after immersion of the dosage forms in the dissolution fluid. The terminal expansion time, tₑₓₚ=4.5, 6, and 7.5 hours for dosage forms A, B, and C;

FIG. 33 plots the elastic modulus of non-limiting experimental dosage forms expanded up to t=tₑₓₚ versus nominal volume fraction of the coating in the solid dosage forms. The expansion time, tₑₓₚ=4.5, 6, and 7.5 hours for dosage forms A, B, and C;

FIG. 34 plots load intensity at fracture, P_{f,df}, and fracture strength, f_{df}, of non-limiting experimental dosage forms expanded up to a time t=tₑₓₚ versus nominal volume fraction of the coating, (φ_{c,n}: (a) P_{f,df} versus φ_{c,n} and (b) of g versus φ_{c,n}. The expansion time, tₑₓₚ=4.5, 6, and 7.5 hours for dosage forms A, B, and C;

FIG. 35 plots load intensity at fracture, P_{f,df}, and tensile strength, f_{df}, versus time after expansion of the dosage forms, t−tₑₓₚ. The fit equations are A: P_{f,df}=−1.46×10⁻²(t−tₑₓₚ)+0.83; σ_{f,df}=−3.86×10⁻⁴(t−tₑₓₚ)+0.022, B: P_{f,df}=−2.99×10⁻²(t−tₑₓₚ)+1.36; f_{df}=−7.93×10⁻⁴(t−tₑₓₚ)+0.036, and C: P_{f,df}==−6.48×10⁻²(t−tₑₓₚ)+2.56; σ_{f,df}=−1.72×10³−(t−tₑₓₚ)+0.068, Table 2. t is the time after immersion of the dosage forms in the dissolution fluid. The expansion times, tₑₓₚ=4.5, 6, and 7.5 hours for dosage forms A, B, and C;

FIG. 36 illustrates position, shape and size of non-limiting experimental dosage form A after administration to a pig. The pig always had access to food and water before and during the experiment. The images were obtained by biplanar fluoroscopy. They show the abdomen in lateral projection (cranial left, caudal right);

FIG. 37 depicts position, shape and size of non-limiting experimental dosage form B after administration to a pig. The pig always had access to food and water before and during the experiment. The images were obtained by biplanar fluoroscopy. They show the abdomen in lateral projection (cranial left, caudal right).

FIG. 38 depicts position, shape and size of non-limiting experimental dosage form C after administration to a pig. The pig always had access to food and water before and during the experiment. The images were obtained by biplanar fluoroscopy. They show the abdomen in lateral projection (cranial left, caudal right).

FIG. 39 presents the normalized expansion of dosage form radius, ΔR_{df}/R_{df,0}, versus time, t, after administration to the pigs: (a) measured data over the entire range, and (b) data truncated to the region t=[0, 1000] min. The fit equations in FIG. 13b are A: ΔR_{df}/R_{df,0}=1.98×10⁻³ t, B: ΔR_{df}/R_{df,0}=1.26×10⁻³ t, and C: ΔR_{df}/R_{df,0}=0.86×10⁻³ t;

FIG. 40 plots static fatigue strength of non-limiting experimental dosage forms. The data points are from FIG. 35;

FIG. 41 plots gastric residence time of non-limiting experimental dosage forms versus nominal volume fraction of the coating;

FIG. 42 presents viscous creep of non-limiting experimental, acidic water-soaked tensile specimen films of Eudragit L100-55: (a) strain, ΔL/L₀, versus time, t, and (b) strain rate, dε/dt, versus stress, σ. The stress was derived as: σ=F/Wh where F is the weight of the applied load, W the width of the thin section of the specimen film, and h its thickness. The fit equations in FIG. 42a are as follows. I: ΔL/L₀=1.087 t, II: ΔL/L₀=0.45 t, III: ΔL/L₀=0.27 t, and IV: ΔL/L₀=0.145 t.

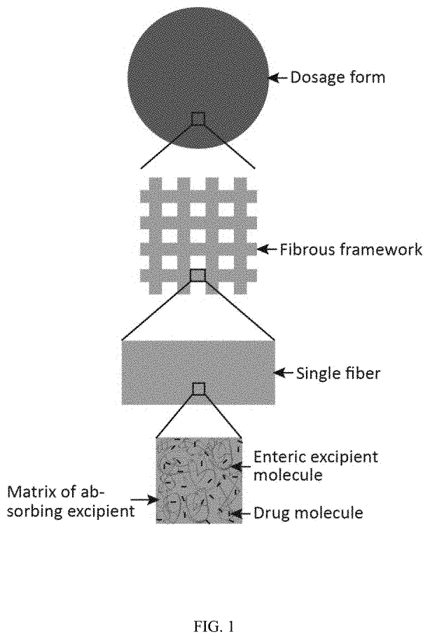

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Moreover, in the disclosure herein, the terms “one or more active ingredients” and “drug” are used interchangeably. As used herein, an “active ingredient” or “active agent” or “drug” refers to an agent whose presence or level correlates with elevated level or activity of a target, as compared with that observed absent the agent (or with the agent at a different level). In some embodiments, an active ingredient is one whose presence or level correlates with a target level or activity that is comparable to or greater than a particular reference level or activity (e.g., that observed under appropriate reference conditions, such as presence of a known active agent, e.g., a positive control).

In the invention herein, a drug-containing solid generally comprises a solid that includes or contains at least a drug. A drug-containing solid generally can have any shape, geometry, or form.

Furthermore, in the context of some embodiments herein, a three dimensional structural framework (or network) of one or more elements comprises a structure (e.g., an assembly or an assemblage or an arrangement or a skeleton or a skeletal structure or a three-dimensional lattice structure of one or more drug-containing elements) that extends over a length, width, and thickness greater than 100 μm. This includes, but is not limited to structures that extend over a length, width, and thickness greater than 200 μm, or greater than 300 μm, or greater than 500 μm, or greater than 700 μm, or greater than 1 mm, or greater than 1.25 mm, or greater than 1.5 mm, or greater than 2 mm.

In other embodiments, a three dimensional structural framework (or network) of elements may comprise a structure (e.g., an assembly or an assemblage or a skeleton or a skeletal structure of one or more elements) that extends over a length, width, and thickness greater than the average thickness of at least one element (or at least one segment) in the three dimensional structural framework (or network) of elements. This includes, but is not limited to structures that extend over a length, width, and thickness greater than 1.5, or greater than 2, or greater than 2.5, or greater than 3, or greater than 3.5, or greater than 4 times the average thickness of at least one element (or at least one segment) in the three dimensional structural framework (or network) of elements.

In some embodiments, a three dimensional structural framework (or network) of elements is continuous. Furthermore, in some embodiments, one or more elements or segments thereof are bonded to each other or interpenetrating.

It may be noted that the terms “three dimensional structural network”, “three dimensional structural framework”, and “three dimensional lattice structure” are used interchangeably herein. Also, the terms “three dimensional structural framework of drug-containing elements”, “three dimensional structural framework of elements”, “three dimensional structural framework of one or more elements”, “three dimensional framework of elements”, “three dimensional structural framework of fibers”, “three dimensional framework”, “structural framework”, etc. are used interchangeably herein.

In the invention herein, a “structural element” or “element” refers to a two-dimensional element (or 2-dimensional structural element), or a one-dimensional element (or 1-dimensional structural element), or a zero-dimensional element (or 0-dimensional structural element).

As used herein, a two-dimensional structural element is referred to as having a length and width much greater than its thickness. In the present disclosure, the length and width of a two-dimensional structural element are greater than 2 times its thickness. An example of such an element is a “sheet”. A one-dimensional structural element is referred to as having a length much greater than its width and thickness. In the present disclosure, the length of a one-dimensional structural element is greater than 2 times its width and thickness. An example of such an element is a “fiber”. A zero-dimensional structural element is referred to as having a length and width of the order of its thickness. In the present disclosure, the length and width of a zero-dimensional structural element are no greater than 2 times its thickness. Furthermore, the thickness of a zero-dimensional element is less than 2.5 mm. Examples of such zero-dimensional elements are “particles” or “beads” and include polyhedra, spheroids, ellipsoids, or clusters thereof.

Moreover, in the invention herein, a segment of a one-dimensional element is a fraction of said element along its length. A segment of a two-dimensional element is a fraction of said element along its length and/or width. A segment of a zero-dimensional element is a fraction of said element along its length and/or width and/or thickness. The terms “segment of a one-dimensional element”, “fiber segment”, “segment of a fiber”, and “segment” are used interchangeably herein. Also, the terms “segment of a two-dimensional element” and “segment” are used interchangeably herein. Also, the terms “segment of a zero-dimensional element” and “segment” are used interchangeably herein.

As used herein, the terms “fiber”, “fibers”, and “one or more fibers”, are used interchangeably. They are understood as the solid, structural elements (or building blocks) that make up part of or the entire three dimensional structural framework or network (e.g., part of or the entire dosage form structure, or part of or the entire structure of a drug-containing solid, etc.). A fiber has a length much greater than its width and thickness. In the present disclosure, a fiber is referred to as having a length greater than 2 times its width and thickness (e.g., the length is greater than 2 times the fiber width and the length is greater than 2 times the fiber thickness). This includes, but is not limited to a fiber length greater than 3 times, or greater than 4 times, or greater than 5 times, or greater than 6 times, or greater than 8 times, or greater than 10 times, or greater than 12 times the fiber width and thickness. In other embodiments that are included but not limiting in the disclosure herein, the length of a fiber may be greater than 0.3 mm, or greater than 0.5 mm, or greater than 1 mm, or greater than 2.5 mm.

Moreover, as used herein, the term “fiber segment” or “segment” refers to a fraction of a fiber along the length of said fiber.

In the invention herein, fibers (or fiber segments) may be bonded, and thus they may serve as building blocks of “assembled structural elements” with a geometry different from that of the original fibers. Such assembled structural elements include two-dimensional elements, one-dimensional elements, or zero-dimensional elements.

In the invention herein, drug release from a solid element (or a solid dosage form, or a solid matrix, or a drug-containing solid) refers to the conversion of drug (e.g., one or more drug particles, or drug molecules, or clusters thereof, etc.) that is/are embedded in or attached to the solid element (or the solid dosage form, or the solid matrix, or three dimensional structural framework, or the drug-containing solid) to drug in a dissolution medium.

As used herein, the terms “dissolution medium”, “physiological fluid”, “body fluid”, “dissolution fluid”, “medium”, “fluid”, “aqueous solution”, and “penetrant” are used interchangeably. They are understood as any fluid produced by or contained in a human body under physiological conditions, or any fluid that resembles a fluid produced by or contained in a human body under physiological conditions. Generally, a dissolution fluid contains water and thus may be aqueous. Examples include, but are not limited to: water, saliva, stomach fluid, gastrointestinal fluid, saline, etc. at a temperature of 37° C. and a pH value adjusted to the relevant physiological condition.

In the invention herein, moreover, a “relevant physiological fluid” is understood as the relevant physiological fluid surrounding the dosage form in the relevant physiological application. For example, if the dosage form is a gastroretentive dosage form, a relevant physiological fluid is gastric fluid.

Furthermore, in the invention herein, a “fluid-absorptive excipient” is referred to as an excipient that is “absorptive” of gastric or a relevant physiological fluid under physiological conditions. Generally, said absorptive excipient is a solid, or a semi-solid, or a viscoelastic material in the dry state at room temperature. Upon contact with (e.g., immersion in) gastric or a relevant physiological fluid under physiological conditions, however, said absorptive excipient can absorb said fluid and form solutions or mixtures with said fluid having a weight fraction of gastric or relevant physiological fluid greater than 0.4. This includes, but is not limited to the formation of solutions or mixtures with a weight fraction of gastric or relevant physiological fluid greater than 0.5, or greater than 0.6, or greater than 0.7, or greater than 0.75, or greater than 0.8, or greater than 0.85, or greater than 0.9, or greater than 0.95. In other words, the solubility of gastric fluid or a relevant physiological fluid in the absorptive excipient under physiological conditions generally is greater than about 400 mg/ml. This includes, but is not limited to solubility of gastric or relevant physiological fluid in an absorptive excipient greater than 500 mg/ml, or greater than 600 mg/ml, or greater than 700 mg/ml, or greater than 750 mg/ml, or greater than 800 mg/ml, or greater than 850 mg/ml, or greater than 900 mg/ml, or greater than 950 mg/ml. Preferably, absorptive excipient is mutually soluble with a relevant physiological fluid. Non-limiting examples of preferred absorptive, high-molecular-weight excipients may include, but are not limited to water-soluble polymers of large molecular weight and with amorphous molecular structure, such as hydroxypropyl methylcellulose with a molecular weight greater than 50 kg/mol or hydroxypropyl methylcellulose with a molecular weight in the range between 50 kg/mol and 300 kg/mol. The terms “physiological fluid-absorptive excipient”, “absorptive excipient”, “fluid-absorptive excipient”, and “water-absorptive excipient” are used interchangeably herein.

In the invention herein, moreover, a “strengthening excipient”, too, generally is a solid, or a semi-solid, or a viscoelastic material in the dry state at room temperature. Upon contact with (e.g., immersion in) gastric or a relevant physiological fluid under physiological conditions, however, said strength-enhancing excipient is far less absorptive of said fluid, and thus it remains a semi-solid, or viscoelastic, or highly viscous material. Generally, the solubility of gastric or relevant physiological fluid in strength-enhancing excipient under physiological conditions is no greater than 800 mg/ml. This includes, but is not limited to a solubility of gastric or a relevant physiological fluid in strength-enhancing excipient under physiological conditions no greater than 750 mg/ml, or no greater than 700 mg/ml, or no greater than 650 mg/ml, or no greater than 600 mg/ml, or no greater than 550 mg/ml, or no greater than 500 mg/ml, or no greater than 450 mg/ml, or no greater than 400 mg/ml. In the non-limiting extreme case, the relevant physiological fluid can be insoluble or practically insoluble in the strength-enhancing excipient.

Typically, however, a relevant physiological fluid is sparingly-soluble in a strengthening excipient. Thus, upon immersion of said strengthening excipient in said relevant physiological fluid, the stiffness (e.g., the elastic modulus) or the viscosity of said strengthening excipient may decrease somewhat compared with the stiffness or viscosity of the dry strengthening excipient. Similarly, upon immersion of strengthening excipient in a relevant physiological fluid, the strain at fracture of said strengthening excipient may increase compared with the strain at fracture of the dry strengthening excipient. Because the strengthening excipient can be a viscoelastic, semi-solid, or highly viscous material even after prolonged immersion in a relevant physiological fluid, it is also referred to herein as “stabilizing excipient”, or “viscoelastic excipient”.

In the invention herein, moreover, the term “mechanically strengthening surface layer”, also referred to as “strength-enhancing surface layer” or “strengthening surface layer”, is generally understood as a membrane, layer, film, coating, coating film, etc. attached to a core. Upon exposure to a relevant physiological fluid, the mechanical properties, such as elastic modulus, yield strength, tensile strength, viscosity, and so on, of said surface layer-supported core (e.g., said core with attached surface layer) are generally greater than the mechanical properties of said core without any mechanically strengthening surface layer. Typically, upon exposure to a relevant physiological fluid, at least a mechanical property, such as elastic modulus, yield strength, tensile strength, viscosity, and so on, of said surface layer-supported core (e.g., said core with attached surface layer) is generally at least two times greater than the corresponding mechanical property of said core without any mechanically strengthening surface layer. This includes, but is not limited to at least a mechanical property, such as elastic modulus, yield strength, tensile strength, viscosity, and so on, of said surface layer-supported core (e.g., said core with attached surface layer) at least three, or at least four, or at least five or at least six, or at least seven, or at least eight times, or at least nine, or at least ten times greater than the corresponding mechanical property of said core without any mechanically strengthening surface layer.

In the invention herein, furthermore, the term “semi-permeable surface layer” is generally understood as a membrane, layer, film, coating, coating film, etc. through which physiological fluid (e.g., water or water molecules) can fairly readily (e.g., fairly easily, fairly rapidly, etc.) pass upon exposure to said physiological fluid, but through which passage of at least an absorptive excipient is hindered or slow or slowed down. Thus, a “semi-permeable surface layer” is generally referred to as a membrane through which the diffusivity of physiological fluid is substantially greater than the diffusivity of a fluid-absorptive excipient. Typically, upon exposure of a semi-permeable surface layer to a physiological fluid (e.g., water, saliva, gastric fluid, etc.) the diffusivity of said fluid through said surface layer is at least 2-5 times greater than the diffusivity of a fluid-absorptive excipient through said surface layer. This includes, but is not limited to diffusivity of physiological fluid through a semi-permeable surface layer at least 5-10 times, or at least 10 times, or at least 20 times, or at least 50 times greater than diffusivity of a fluid-absorptive excipient through said semi-permeable surface layer.

In the invention herein, a material (e.g., a membrane, a composite mass, etc) is generally referred to as “viscoelastic” if it exhibits both viscous and elastic characteristics when undergoing deformation. By way of example but not by way of limitation, upon exposure of a viscoelastic material to a small stress or load for a short time, said viscoelastic material may behave similar to an elastic solid and spring back after unloading. If the viscous material is exposed to said small stress or load for a long time, however, said viscoelastic material may behave more like a highly viscous mass and deform plastically. An estimate of the “critical time” (e.g., the loading time below which a viscoelastic material may behave more like an elastic solid and above which said viscoelastic material may exhibit substantial plastic deformation) is the “relaxation time” defined as the ratio of elongational viscosity and elastic modulus of the material. Typically, as used herein the relaxation time of a viscoelastic material is greater than about 0.1-0.5 seconds, and more preferably greater than about a second, and even more preferably greater than about 2-5 seconds. Also, upon loading and unloading a viscoelastic material the stress-strain curve of said viscoelastic material may exhibit a hysteresis loop. A non-limiting example of a viscoelastic material is rubber, such as natural rubber.

In the invention herein, moreover, a core may generally be referred to as “substantially encapsulated” by a surface layer if said surface layer covers (e.g., encloses, coats, etc.) at least 20 percent of the surface of said core. This includes, but is not limited to said surface layer covering at least 30 percent, or at least 40 percent, or at least 50 percent, or at least 60 percent, or at least 70 percent, or at least 80 percent, or at least 90 percent, or about 100 percent of the surface of said core.

Further information related to the definition, characteristics, features, composition, analysis etc. of the disclosed dosage forms, and the elements for fabricating or constructing them, is provided throughout this specification.

### SCOPE OF THE INVENTION

It is contemplated that a particular feature described either individually or as part of an embodiment in this disclosure can be combined with other individually described features, or parts of other embodiments, even if the other features and embodiments make no mention of the particular feature. Thus, the invention herein extends to such specific combinations not already described. Furthermore, the drawings and embodiments of the invention herein have been presented as examples, and not as limitations. Thus, it is to be understood that the invention herein is not limited to these precise embodiments. Other embodiments apparent to those of ordinary skill in the art are within the scope of what is claimed.

By way of example but not by way of limitation, it is contemplated that compositions, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Furthermore, where compositions, articles, and devices are described as having, including, or comprising specific components, or where processes and methods are described as having, including, or comprising specific steps, it is contemplated that, additionally, there are compositions, articles, and devices of the present invention that consist essentially of, or consist of, the recited components, and that there are processes and methods according to the present invention that consist essentially of, or consist of, the recited processing steps.

Similarly, where compositions, articles, and devices are described as having, including, or comprising specific compounds and/or materials, it is contemplated that, additionally, there are compositions, articles, and devices of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. Headers are provided for organizational purposes and are not meant to be limiting

### DETAILED DESCRIPTION OF THE INVENTION

### Aspects of the Dosage Form

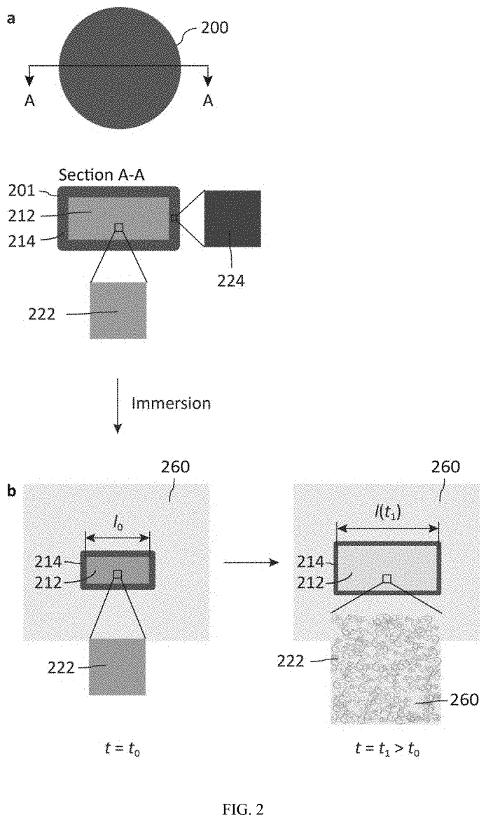

As shown schematically in the non-limiting FIG. 2a, the dosage forms 200 disclosed herein generally comprise a drug-containing solid 201 having a physiological fluid-absorptive solid core 212, also referred to herein as “fluid-absorptive solid core”, “fluid-absorptive core”, “solid core”, or “core”, and a mechanically strengthening, semi-permeable surface layer 214. The fluid-absorptive core 212 generally comprises at least a fluid-absorptive first excipient 222. The fluid-absorptive core 212 further is substantially encapsulated (e.g., substantially coated, substantially surrounded, etc.) by the mechanically strengthening, semi-permeable surface layer 214. The semi-permeable surface layer 214 generally comprises at least a mechanically strengthening second excipient 224. As shown in the non-limiting FIG. 2b, upon exposure of the dosage form 200 or drug-containing solid 201 to physiological fluid 260, the surface layer 214 encapsulated solid core 212, 222 (or surface layer 214 supported solid core 212, 222) expands with fluid 260 absorption.

In preferred embodiments, moreover, upon exposure of the dosage form 200 or drug-containing solid 201 to a physiological fluid 260, the surface layer-encapsulated solid core 212, 222 expands primarily with fluid 260 absorption. In the invention herein, a solid is generally understood as “expanding primarily with fluid absorption” if upon exposure of said solid to a physiological fluid, the greatest expansion of said solid (e.g., the greatest longitudinal expansion, such as the greatest increase in length or normalized length; the greatest volumetric expansion, such as the greatest increase in volume or normalized volume; etc.) is mostly or primarily due to the absorption of said physiological fluid. It may be noted that the surface layer-encapsulated solid core 212, 222 may generally transition to a viscous (e.g., a highly viscous) or semi-solid mass as it expands with fluid 260 absorption.

In preferred embodiments, upon exposure of the dosage form 200 or drug-containing solid 201 to a physiological fluid 260, the mechanically strenghtening, semi-permeable surface layer 214 forms a semi-permeable, viscoelastic membrane.

In preferred embodiments, moreover, upon exposure of the dosage form 200 or drug-containing solid 201 to a physiological fluid 260, a mechanically strengthening, semi-permeable surface layer 214 is substantially permeable to said physiological fluid 260. In the invention herein, a membrane or layer is generally referred as “substantially permeable” to a physiological fluid if the diffusivity of water in said membrane or layer is greater than about 0.005 times the self-diffusivity of water. Thus, generally, in the invention herein a membrane or layer is understood “substantially permeable” to a physiological fluid if the diffusivity of water in said membrane or layer under physiological conditions (e.g., at a temperature of 37° C.) is greater than about 0.5×10⁻¹¹ m²/s.

Similarly, in preferred embodiments, upon exposure of the dosage form 200 or drug-containing solid 201 to a physiological fluid 260, said mechanically strengthening, semi-permeable surface layer 214 is substantially impermeable to at least one fluid-absorptive first excipient 222. In the invention herein, a membrane or layer is generally understood “substantially impermeable” to a fluid-absorptive first excipient if a diffusivity of said fluid-absorptive first excipient in or through said membrane or layer is smaller than 0.1 times the diffusivity of water in or through said membrane or layer. This includes, but is not limited to a diffusivity of said fluid-absorptive first excipient in or through said membrane or layer smaller than 0.05 times, or smaller than 0.02 times, or smaller than 0.01 times, or smaller than 0.005 times the diffusivity of water in or through said membrane or layer.

In preferred embodiments, moreover, upon exposure of the dosage form 200 or drug-containing solid 201 to a physiological fluid 260, the mechanically strenghtening, semi-permeable surface layer 214 expands due to an internal pressure in the core 212, said internal pressure generated by osmotic flow of fluid 260 into said core 212.

Generally, furthermore, upon exposure of the dosage form 200 or drug-containing solid 201 to a physiological fluid 260, the drug-containing solid 201 forms an expanded, viscoelastic composite mass 205.

It may be noted, furthermore, that in preferred embodiments, the solid core 212 may comprise a mixture of a drug and at least one fluid-absorptive first excipient 222.

In preferred embodiments, moreover a solid core 212 generally has at least one dimension (e.g., a length, width, or thickness) greater than 3 mm.

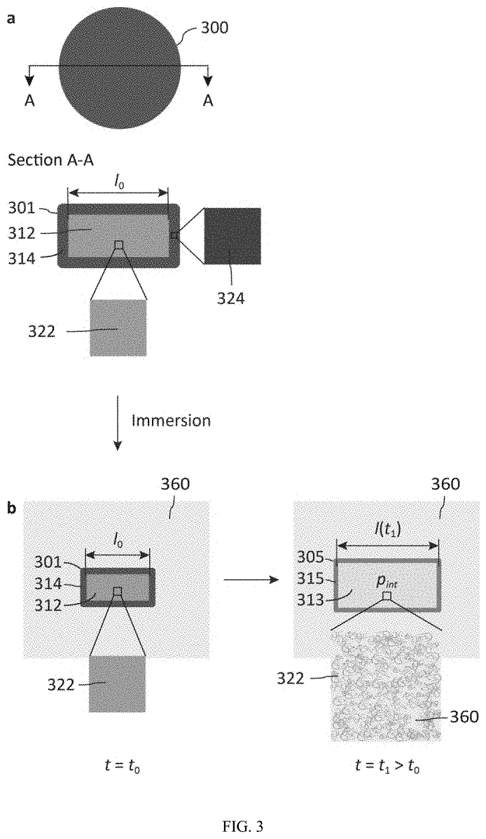

Another non-limiting pharmaceutical dosage form according to the invention herein is shown in FIG. 3a. The dosage form 300 comprises a drug-containing solid 301 having a fluid-absorptive solid core 312 and a mechanically strengthening, semi-permeable surface layer 314. The fluid-absorptive solid core 312 comprises at least a first excipient 322, said first excipient 322 includes at least a fluid-absorptive polymer 322. The fluid-absorptive solid core 312 further is substantially encapsulated (e.g., substantially coated, substantially surrounded, etc.) by the mechanically strengthening, semi-permeable surface layer 314. The semi-permeable surface layer 314 comprises at least a second excipient 324, wherein said second excipient 324 includes at least a mechanically strengthening polymer 324. As shown schematically in the non-limiting FIG. 3b, upon exposure of the dosage form 300 or drug-containing solid 301 to a physiological fluid 360, the surface layer 312 encapsulated solid core 312 expands primarily with fluid 360 absorption, thereby transitioning to a viscous or semi-solid mass 313. Additionally, the mechanically strenghtening, semi-permeable surface layer 314 forms a semi-permeable, viscoelastic membrane 315. The semi-permeable, viscoelastic membrane expands 314, 315 due to an internal pressure, pᵢₙₜ, in the core 312, 313 generated by osmotic flow of fluid 360 into said core 312, 313. Furthermore, upon exposure to said physiological fluid the drug-containing solid 301 forms an expanded, viscoelastic composite mass 305 having a length (e.g., l(t₁)) greater than 1.3 times its length prior to exposure to said physiological fluid (e.g., l₀).

FIG. 3a presents a further non-limiting pharmaceutical dosage form according to the invention herein. The dosage form 300 comprises: a drug-containing solid 301 having a fluid-absorptive solid core 312 and a mechanically strengthening, semi-permeable surface layer 314. The fluid-absorptive solid core 312 has at least a dimension (e.g., l₀) greater than 3 mm. The fluid-absorptive solid core 312 further comprises at least a first excipient 322, said first excipient 322 includes at least a fluid-absorptive polymer 322. The fluid-absorptive solid core 312 further is substantially encapsulated by (e.g., substantially coated, substantially surrounded, etc.) the mechanically strengthening, semi-permeable surface layer 314. The semi-permeable surface layer 314 comprises at least a second excipient 324, wherein said second excipient 324 includes at least a mechanically strengthening polymer 324. As shown schematically in the non-limiting FIG. 3b, upon exposure of the dosage form 300 or drug-containing solid 301 to a physiological fluid 360, the surface layer 312 encapsulated solid core 312 expands primarily with fluid 360 absorption, thereby transitioning to a viscous or semi-solid mass 313. Additionally, the mechanically strenghtening, semi-permeable surface layer 314 forms a semi-permeable, viscoelastic membrane 315. The semi-permeable, viscoelastic membrane expands 314, 315 due to an internal pressure, pᵢₙₜ, in the core 312, 313 generated by osmotic flow of fluid 360 into said core 312, 313. Consequently, upon exposure to a physiological fluid the drug-containing solid 301 forms an expanded, viscoelastic composite mass 305.

FIG. 3a presents yet another non-limiting pharmaceutical dosage form according to the invention herein. The dosage form 300 comprises: a drug-containing solid 301 having a fluid-absorptive solid core 312 and a mechanically strengthening, semi-permeable surface layer 314. The fluid-absorptive solid core 312 has at least a dimension (e.g., l₀) greater than 3 mm. The fluid-absorptive solid core 312 further comprises at least a first excipient 322, said first excipient 322 includes at least a fluid-absorptive polymer 322. The fluid-absorptive solid core 312 further is substantially encapsulated (e.g., substantially coated, substantially surrounded, etc.) by the mechanically strengthening, semi-permeable surface layer 314. The semi-permeable surface layer 314 comprises at least a second excipient 324, wherein said second excipient 324 includes at least a mechanically strengthening polymer 324. As shown schematically in the non-limiting FIG. 3b, upon exposure of the dosage form 300 or drug-containing solid 301 to a physiological fluid 360, the surface layer 312 encapsulated solid core 312 expands primarily with fluid 360 absorption, thereby transitioning to a viscous or semi-solid mass 313. Additionally, the mechanically strenghtening, semi-permeable surface layer 314 forms a semi-permeable, viscoelastic membrane 315. The semi-permeable, viscoelastic membrane expands 314, 315 due to an internal pressure, pᵢₙₜ, in the core 312, 313 generated by osmotic flow of fluid 360 into said core 312, 313. Furthermore, within no more than 10 hours of exposure to said physiological fluid the drug-containing solid 301 forms an expanded, viscoelastic composite mass 305 having a length (e.g., l(t₁)) between 1.3 and 5 times its length prior to exposure to said physiological fluid (e.g., l₀).

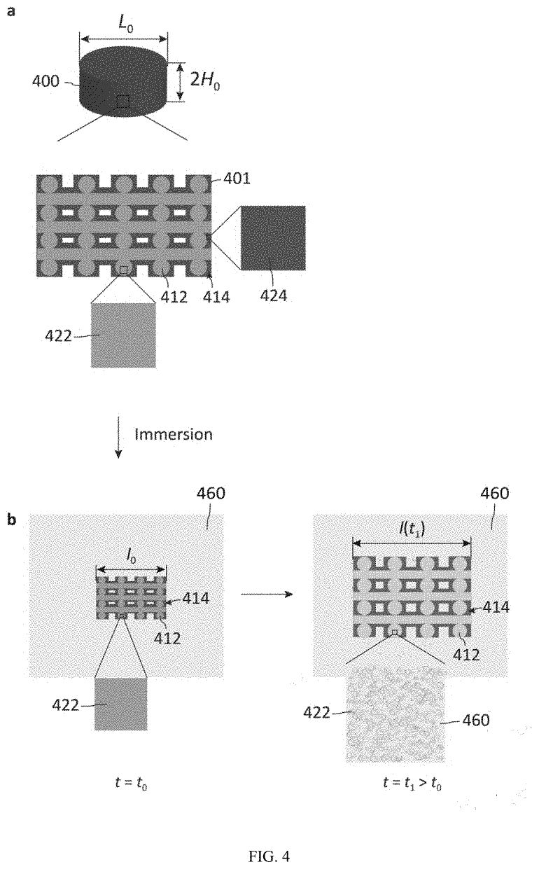

Another non-limiting pharmaceutical dosage form according to the invention herein is shown in FIG. 4a. The dosage form 400 comprises a drug-containing solid 401 having a fluid-absorptive solid core 412 and a mechanically strengthening, semi-permeable surface layer 414. The fluid-absorptive solid core 412 comprises a three-dimensional structural framework of structural elements 412. The structural elements 412 comprise at least a fluid-absorptive first excipient 422. The structural elements 412 are further substantially encapsulated (e.g., substantially coated, substantially surrounded, etc.) by said mechanically strengthening, semi-permeable surface layer 414. The semi-permeable surface layer 414 comprises at least a mechanically strengthening second excipient 424. As shown schematically in the non-limiting FIG. 4b, upon exposure of the dosage form 400 or drug-containing solid 401 to physiological fluid 460, the surface layer 414 supported structural framework 412 expands with fluid 460 absorption.

FIG. 4a presents a further non-limiting pharmaceutical dosage form according to the invention herein. The dosage form 400 comprises a drug-containing solid 401 having a fluid-absorptive solid core 412 and a mechanically strengthening, semi-permeable surface layer 414. The fluid-absorptive solid core 412 comprises a three-dimensional structural framework (or network) of criss-crossed stacked layers of fibers 412. The fibers 412 comprise at least a fluid-absorptive first excipient 422. The fibers 412 are further substantially encapsulated (e.g., substantially coated, substantially surrounded, etc.) by said mechanically strengthening, semi-permeable surface layer 414. The semi-permeable surface layer 414 comprises at least a mechanically strengthening second excipient 424. As shown schematically in the non-limiting FIG. 4b, upon exposure of the dosage form 400 or drug-containing solid 401 to physiological fluid 460, the surface layer 414 supported structural framework 412 expands with fluid 460 absorption.

In some preferred embodiments, moreover, a surface layer supported solid core (e.g., a surface layer supported three dimensional structural framework of elements, a three-dimensional structural framework (or network) of criss-crossed stacked layers of fibers, etc.) and/or a drug-containing solid may expand in all dimensions with fluid absorption. The terms “expanding in all dimensions”, “expand in all dimensions”, or “expansion in all dimensions” are generally understood as an increase in a length of a sample (e.g., the length, and/or width, and/or thickness, etc. of said sample) and an increase in volume of said sample. Thus, pure shear deformation is not considered “expansion in all dimensions” herein.

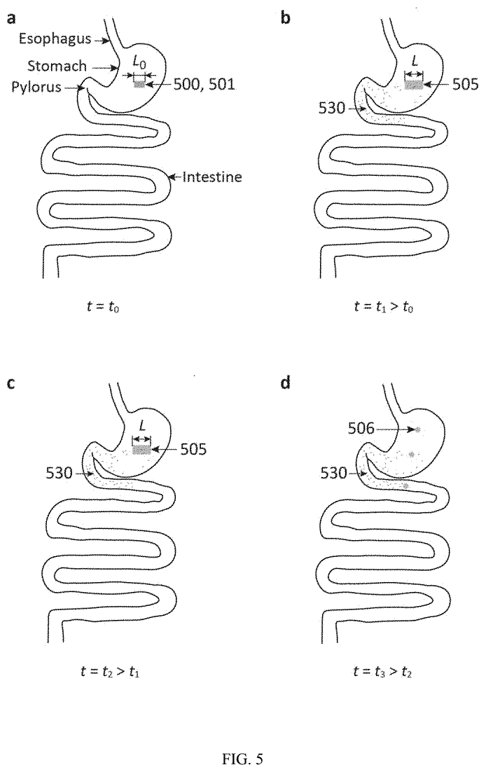

FIG. 5 presents a non-limiting course of a dosage form 500 (or a drug-containing solid 501) after ingestion by a human or animal subject (e.g., a dog, a pig, etc.). Initially, the dosage form 500 is solid and has a swallowable size and geometry. Upon ingestion, the dosage form 500 enters the stomach, and the drug-containing solid 500, 501 expands with fluid absorption. As a result, a viscoelastic mass 505 is formed with a size (e.g., a width, diameter, etc.) greater than the diameter or width of the pylorus and a strength or stiffness so large that it is substantially unfragmentable in the gastric environment (e.g., under normal gastric conditions) for prolonged time, FIG. 5b.

Moreover, drug molecules 530 may be released from the drug-containing solid 500, 501 or the viscoelastic mass 505 into the gastric fluid over prolonged time, FIGS. 5b and 5c. Thus, because the size and the strength or stiffness of the viscoelastic mass 505 may remain sufficiently large to prevent its passage through the pylorus into the intestines for prolonged time, drug 530 release into the stomach can be prolonged and/or controlled. Eventually, however, the stiffness or strength of the viscoelastic mass 505, 506 may be so low that it disintegrates, or deforms excessively, or breaks up, or fragments, or dissolves, etc. in the stomach. The fragments 506 may pass into the intestines, FIG. 5d. It may be noted that the terms “disintegrate” or “disintegration” are used as equivalents to “fragment”, “fragmentation”, “deform”, “excessive deformation”, “dissolve”, “dissolution”, “erode”, “erosion”, “mechanically weaken”, “soften”, “break up”, “rupture”, and so on.

Additional aspects and embodiments of dosage forms disclosed herein are described throughout this specification. Any more aspects and embodiments that would be obvious to a person of ordinary skill in the art are all within the spirit and scope of this invention.

### Models of Expansion, Mechanical Properties, and Gastric Residence Time of Dosage Forms

This section presents non-limiting ways by which the expansion, mechanical properties, and gastric residence time of disclosed dosage forms may be modeled. The models and examples will enable one of skill in the art to more readily understand the conceptual details and advantages of the invention. The models and examples are for illustrative purposes only, and are not meant to be limiting in any way.

### (a) Dosage Form Microstructures and Formulation

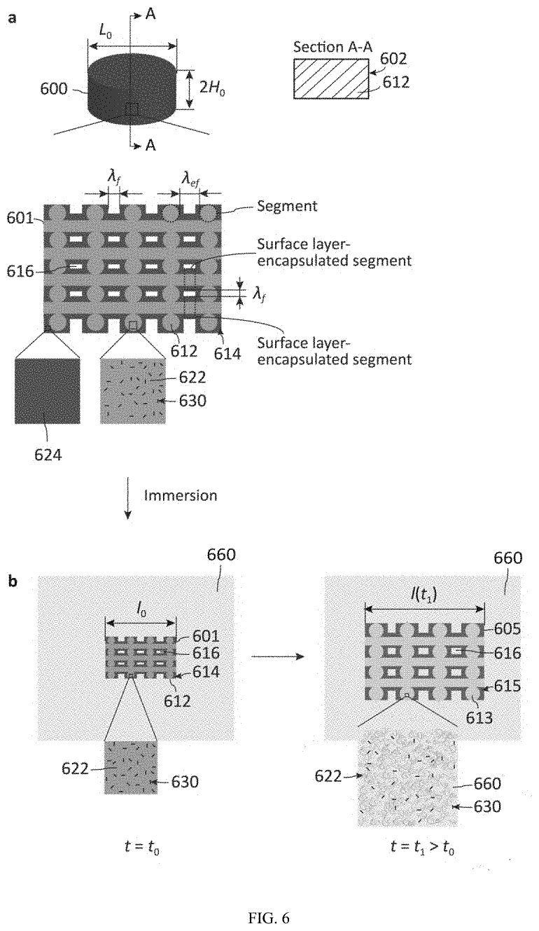

The non-limiting models refer to dosage forms as shown schematically in the non-limiting FIG. 6a. The dosage forms 600 comprise a drug-containing solid 601 having a fluid-absorptive solid core 612 and a mechanically strengthening, semi-permeable surface layer 614. The fluid-absorptive solid core 612 comprises a three-dimensional structural framework (or network) of criss-crossed stacked layers of fibers 612. The fibers 612 comprise a mixture of at least a drug 630 and a fluid-absorptive first excipient 622. The fibers 612 are further substantially encapsulated (e.g., substantially coated, substantially surrounded, etc.) by said mechanically strengthening, semi-permeable surface layer 614. The semi-permeable surface layer 614 comprises at least a mechanically strengthening second excipient 624. The surface layer-encapsulated fibers 612, 614 (e.g., the fibers 612 and the surface layer 614 combined) further comprise surface layer-encapsulated segments spaced apart from adjoining surface layer-encapsulated segments by free spacings, λ_{f}, thereby defining one or more free spaces 616 in the drug-containing solid 601.

In the specific non-limiting dosage forms modeled herein, moreover, the fibers 612 further comprise segments separated and spaced apart from adjoining segments by element-free or fiber-free spacings, λ_{fe}, defining one or more element-free or fiber-free spaces 614, 616 in the drug-containing solid 601. In the specific, non-limiting examples modeled herein the fiber-free space 614, 616 is substantially connected, or substantially contiguous, through the drug-containing solid 601 or through the outer volume of the three dimensional structural framework of fibers 612. Moreover, the free space 616 is filled with a matter comprising a gas, such as air.

Furthermore, in the specific non-limiting dosage forms modeled herein, the physiological fluid-absorptive polymeric excipient 622 generally comprises hydroxypropylmethylcellulose (HPMC) of molecular weight about 120 kg/mol. The weight fraction of said absorptive excipient 622 in the fibers 612 (e.g., the weight fraction of HPMC in the solid core 612) is about 0.42. The volume fraction of said absorptive excipient 622 in the fibers 612 (e.g., the volume fraction of HPMC in the solid core 612) is about 0.46.

In the specific non-limiting dosage forms modeled herein, moreover, the mechanically strengthening, second excipient 624 (e.g., the mechanically strengthening, semi-permeable surface layer 614) comprises methacrylic acid-ethyl acrylate copolymer with a molecular weight of about 250 kg/mol (also referred to herein as “Eudragit L100-55”).

The three dimensional structural framework of fibers 612 further comprises an outer surface 602 and an outer volume defined by the volume enclosed by said outer surface 602. In the non-limiting dosage forms modeled herein, the volume fraction of mechanically strengthening, semi-permeable surface layer 614 (e.g., the volume fraction of mechanically strengthening, second excipient 624) within said outer volume generally is in the range between about 0.025 and about 0.14 (e.g., 0.025 (dosage form A), 0.041 (dosage form B), 0.068 (dosage form C), and 0.14 (dosage form D)). A non-limiting method for estimating or predicting the volume fraction of mechanically strengthening, semi-permeable surface layer 614, 624 in an outer volume is given in experimental example 2.4 titled “Microstructures of dosage forms” herein.

### (b) Expansion

Without wishing to be bound to a particular theory, as shown schematically in the non-limiting FIG. 6b, upon immersion of the dosage form 600 or drug-containing solid 601 in a stirred dissolution fluid 660, such as deionized (DI) water with 0.1 M hydrochloric acid (HCl), said fluid may percolate at least a free space 616 and wet mechanically strengthening surface layer 614 of the structural framework 612. This may allow the fluid 660 to diffuse through mechanically strenghtening, semi-permeable surface layer 614 and into one or more said fibrous elements 612. As a result, the fibrous framework 612, 614 may expand in all dimensions with fluid 660 absorption and transition to a semi-solid or viscous mass 613. Additionally, the mechanically strenghtening, semi-permeable surface layer 614 may form a semi-permeable, viscoelastic membrane 615. The semi-permeable, viscoelastic membrane may expand 614, 615 due to an internal pressure, pᵢₙₜ, in the core 612, 613 generated by osmotic flow of fluid 660 into said core 612, 613. As a result, the drug-containing solid 601 forms an expanded, viscoelastic composite mass 605 having a length (e.g., l(t₁)) between 1.3 and 5 times its length prior to exposure to said physiological fluid (e.g., l₀).

An in-depth analysis of dosage form expansion is far beyond the scope of this paper. Thus, highly approximate engineering models of dosage form expansion are developed based on models of the expansion of coated, single fibers. Two “extreme” cases are considered.

(b1) Expansion of Single Fibers Limited by Diffusion of Water into the Fibers

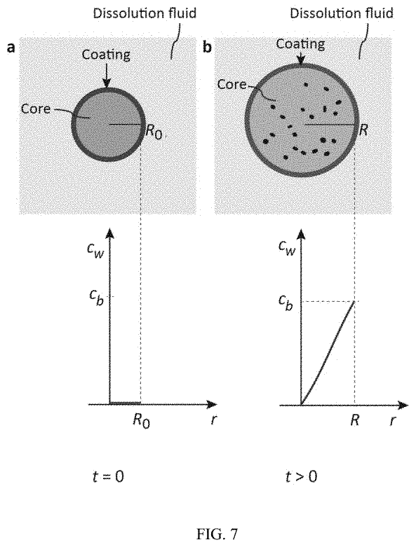

In the first case, the coating is thin and compliant, and the internal pressure is small compared with the osmotic pressure. Fiber expansion may then be limited by diffusion of physiological fluid, or water, into the fiber, as shown schematically in FIG. 7.

An in-depth analysis of the diffusion of water into the expanding fiber is beyond the scope of this paper. As shown in prior work, however, an engineering approximation of the diffusion-limited normalized radial fiber expansion may be written as (for further details, see, e.g., A. H. Blaesi, N. Saka, Solid-solution fibrous dosage forms for immediate delivery of sparingly-soluble drugs: Part 2. Dosage forms by 3D-micro-patterning, Mater. Sci. Eng. C 119, 2021, 110211; A. H. Blaesi, N. Saka, Expandable, dual-excipient fibrous dosage forms for prolonged delivery of sparingly soluble drugs, Int. J. Pharm., 2021, 120396):

$$\frac{Δ {R}_{f}}{{R}_{0}} ≅ \frac{4}{3 \sqrt{π}} \frac{{c}_{b}}{{ρ}_{w}} {( \frac{{D}_{w} t}{{R}_{0}^{2}} )}^{1 / 2} \qquad ( 1 )$$

where R_{f} is the fiber radius at time t, R₀ the initial fiber radius, c_{b} the boundary concentration of water in the fiber, p_{w} the water density, and D, the water diffusivity in the fiber.

Thus, by Eq. (1) a primary parameter to adjust the fiber expansion rate in diffusion-limited expansion is the radius of the solid fiber, R₀. Substituting the non-limiting parameters ρ_{w}/c_{b}˜ 1, D_{w}˜2×10⁻¹¹ m²/s, and R₀=150 μm in Eq. (1), ΔR_{f}/R₀=0.5 in about 8 minutes. By contrast, if R₀ is increased to 1.5 mm and all other parameters remain unchanged, by Eq. (1) about 800 minutes (13.3 hours) would be required to expand the fiber to a normalized radial expansion of 0.5.

For further information related to the diffusion of dissolution fluid into fibers or other geometries, see, e.g., J. Crank, “The Mathematics of Diffusion”, second edition, Oxford University Press, 1975. More models for estimating the expansion rate of the fibers obvious to a person of ordinary skill in the art are all within the spirit and scope of this disclosure.

### (b2) Expansion of Single Fibers Limited by the Strain Rate of the Mechanically Strengthening, Semi-Permeable Surface Layer

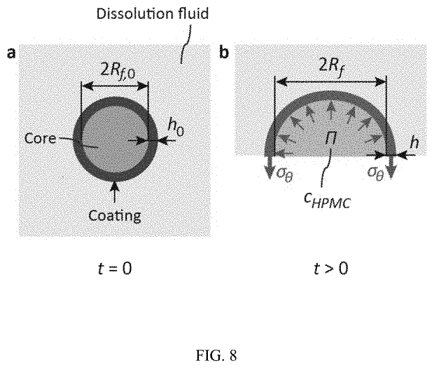

In the second case, the coating is stiffer, and the internal pressure is about equal to the osmotic pressure, Π. The osmotic pressure may induce a tensile stress, σ_{θ}, in the coating that may cause the coating to deform and expand, FIG. 8.

Under the highly approximate assumption that the fluid-penetrated fiber core is a dilute solution of water and HPMC molecules, the osmotic pressure may be written as (for further details, see, e.g., J. van′t Hoff, XII The function of osmotic pressure in the analogy between solutions and gases, Phil. Mag. S.5. 26 (1888) 81-105; G. N. Lewis, The osmotic pressure of concentrated solutions, and the laws of the perfect solution, J. Am. Chem. Soc. 30 (1908) 668-683; P. W. Atkins, Physical Chemistry, 5^{th} edn., Oxford University Press, Oxford, U K, 1994 pp. 227-228, 846-849):

$$Π = \frac{{RTc}_{HPMC}}{{M}_{HPMC}} \qquad ( 2 )$$

where R is the ideal gas constant, T the temperature, M_{HPMC} the molecular weight of HPMC (e.g., the water-absorbing excipient), and c_{HPMC} the concentration of HPMC in the expanding fiber. In isotropic expansion,

$${c}_{HPMC} = \frac{{φ}_{HPMC} {ρ}_{HPMC}}{{( 1 + Δ {R}_{f} / {R}_{0} )}^{3}} \qquad ( 3 )$$

where φ_{HPMC} is the volume fraction of HPMC in the solid fiber core, ρ_{HPMC} the density of solid HPMC, and ΔR/R₀ the normalized radial expansion of the fiber.

Substituting Eq. (3) in Eq. (2) the osmotic pressure can be estimated as:

$$Π = \frac{RT {φ}_{HPMC} {ρ}_{HPMC}}{{( 1 + Δ {R}_{f} / {R}_{0} )}^{3} {M}_{HPMC}} \qquad ( 4 )$$

Substituting non-limiting parameters, R=8.314 J/molK, T=310, φ_{HPMC}=0.46, ρ_{HPMC}=1300 kg/m³, M_{HPMC}=120 kg/mol in Eq. (4), at the normalized radial fiber expansion, ΔR/R₀=0.5 the osmotic pressure, Π=3.81 kPa. Moreover, by Eq. (4) the osmotic pressure could be altered (e.g., increased or decreased) by adjusting (e.g., increasing or decreasing) the volume fraction of absorptive excipient (e.g., HPMC) in the fibers.

The hoop stress in the coating may be estimated from the osmotic pressure by:

$${σ}_{θ} = Π \frac{{R}_{f}}{h} \qquad ( 5 )$$

where h is the coating thickness and R_{f} the radius of the expanding fiber core. The fiber radius and coating thickness may further be related to the volume fractions of core (e.g the volume fraction of fiber core) and the volume fraction of coating:

$$\frac{{φ}_{f}}{{φ}_{c}} = \frac{π {R}_{f}^{2}}{2 π {R}_{f} h} = \frac{{R}_{f}}{2 h} \qquad ( 6 a )$$

$$Thus ,$$

$$\frac{{R}_{f}}{h} = \frac{2 {φ}_{f}}{{φ}_{c}} \qquad ( 6 b )$$

Substituting Π=3.81 kPa and φ_{f}/φ_{c}=27.5, 16.5, and 9.9 (the ratios in the non-limiting experimental dosage forms A, B, and C, Table 5 later) in Eqs. (6) and (5), σ_{θ}=210, 126, and 75 kPa for the fibers in the non-limiting experimental dosage forms A, B, and C.

If the coating is a viscoelastic membrane that creeps or deforms plastically or viscously upon prolonged exposure to a stress, the normalized radial expansion rate of the fibers may be written by an adapted form of Hooke's law as:

$$\frac{1}{{R}_{0}} \frac{{dR}_{f}}{dt} = \frac{{σ}_{θ}}{η} \qquad ( 7 )$$

where J is the elongational viscosity of the coating. Integrating gives:

$$\frac{Δ {R}_{f}}{{R}_{0}} = \frac{{σ}_{θ}}{η} t \qquad ( 8 )$$

Substituting the non-limiting parameters of dosage forms A, B, and C, σ_{θ}=210, 126, and 75 kPa, and η=1.36×10⁸ Pa·s in Eq. (8), ΔR_{f}/R₀=0.5 after about 5.5, 9.5, and 15 minutes.

By Eqs. (4)-(8) the fiber expansion rate could be altered (e.g., increased or decreased) by adjusting (e.g., increasing or decreasing) the elongational viscosity of the coating or the osmotic pressure (e.g., the volume fraction of absorptive excipient (e.g., HPMC)) in the fibers, among others. More models for estimating the expansion rate of the fibers obvious to a person of ordinary skill in the art are all within the spirit and scope of this disclosure.

### (b3) Expansion of Dosage Forms

Because the dosage forms may expand as the fibers expand, the normalized radial or longitudinal expansion of the dosage form, ΔR_{df}/R₀, may be roughly proportional to the normalized radial expansion of the single fibers.

Thus, in diffusion-limited expansion:

$$\frac{Δ {R}_{df}}{{R}_{df , 0}} ≅ {κ}_{1} \frac{4}{3 \sqrt{π}} \frac{{c}_{b}}{{ρ}_{w}} {( \frac{{D}_{w} t}{{R}_{0}^{2}} )}^{1 / 2} \qquad ( 9 )$$

In expansion limited by the strain rate of the coating:

$$\frac{Δ {R}_{df}}{{R}_{df , 0}} = {κ}_{2} \frac{{σ}_{θ} t}{η} \qquad ( 10 )$$

where κ₁ and κ₂ are constants. Non-limiting ranges of the constants, κ₁˜0.5-1 and κ₂˜0.05-1.

Thus, for the non-limiting parameters above, to achieve a normalized radial expansion of the dosage form of 0.5 in less than about 600 min (10 h), by Eq. (9) the fiber radius should be no greater than about 650 μm. Similarly, for the non-limiting stresses in the coating, σ_{θ}=210, 126, and 75 kPa, by Eq. (10) the elongational viscosity of the coating should be no greater than about 7.5×10⁸, 4.5×10⁸, and 3×10⁸ Pa·s (e.g., no greater than about 1×10⁹ Pa·s) to achieve ΔR_{df}/R₀˜0.5 in less than about 600 min (10 h).

It may be noted again that all the above and below calculations are approximate and for illustrative purposes only. None of the numbers should be considered as exact. More models for estimating the expansion rate of dosage forms obvious to a person of ordinary skill in the art are all within the spirit and scope of this disclosure.

### (c) Mechanical Properties of Expanded Dosage Forms

The expanded dosage forms may be a viscoelastic mass that behaves similar to an elastic solid if the load is applied for a short time. An in-depth derivation of the mechanical properties of the expanded dosage forms is again far beyond the scope of this disclosure. The coating over the fibers may, however, enhance the stiffness and strength of the expanded form substantially. Thus, for obtaining highly approximate engineering approximations of the expanded dosage form's mechanical properties, the coating over the fibers may be treated as a “stiff” and “strong” cellular network, and the stiffness and strength of the fiber core may be neglected.

The elastic modulus of the expanded dosage form may then be estimated as (for further details on how the mechanical properties of a cellular structure may be estimated, see, e.g., L. J. Gibson, M. F. Ashby, G. S. Schajer, C. I. Robertson, The mechanics of two-dimensional cellular materials, Proc. R. Soc. Lond. A, 382 (1982) 25-42; M. F. Ashby, The mechanical properties of cellular solids, Metall. Trans. A 14A (1983) 1755-1769; L. J. Gibson, M. F. Ashby, Cellular solids: Structure and properties, second ed. Cambridge University Press, Cambridge, UK, 1997):

E_{df}=C₂Eφ_{c}² (11)

where E is the elastic modulus of the physiological fluid-soaked coating, C₂ is a constant of about unity, and φ_{c} the volume fraction of the coating in the expanded dosage form.

Substituting the non-limiting parameters C₂=1, E=5.7 MPa, and φ_{c}=0.025-0.14 in Eq. (11), the calculated elastic modulus of the expanded dosage forms, E_{df}=0.004 MPa-0.11 MPa (e.g., about 0.004 MPa-0.5 MPa).

Similarly, the fracture strength of the expanded dosage form may be estimated as:

σ_{f,df}=σ_{f}Cₛφ_{c}^{3/2} (12)

where C₈ is a constant and σ_{f} the fracture strength of the physiological fluid-soaked coating. Substituting the non-limiting parameters, σ_{f}=1.8 MPa, Cₛ=0.65, and φ_{ec}=0.025-0.14 in Eq. (12), the fracture strength, σ_{f,df}=0.0046 MPa-0.061 MPa (e.g., about 0.004 MPa-0.5 MPa).

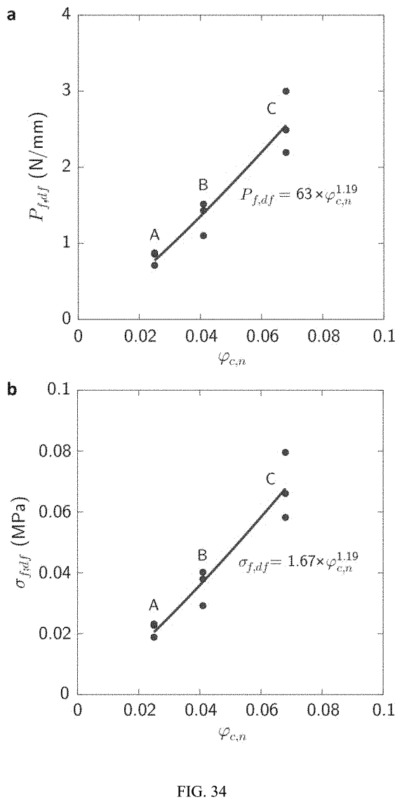

It may be noted that in some embodiments, as shown in the non-limiting FIG. 34 later the fracture strength of the dosage forms may also be estimated as σ_{f,df}=σ_{f}Cₛφ_{c}ⁿ, where m is a constant.

It may be noted again that all the above and below calculations are approximate and for illustrative purposes only. None of the numbers should be considered as exact. More models for estimating the mechanical properties of expanded dosage forms obvious to a person of ordinary skill in the art are all within the spirit and scope of this disclosure.

### (d) Gastric Residence Time of Dosage Forms

As for the previous models, an in-depth analysis of the gastric residence time of expanded dosage forms is far beyond the scope of this disclosure. Two highly approximate, non-limiting engineering models for estimating the gastric residence time are considered below.

### (d1) Dosage Form Disintegration Due to Static Fatigue

In the first, the coating membrane over the expanded fibers deteriorates and weakens due to chemical processes. As a result, the strength of the expanded dosage form may decrease with time. In analogy to the “universal static fatigue curve” of glasses (for further details, see, e.g., R. E. Mould, R. D. Southwick, Strength and static fatigue of abraded glass under controlled ambient conditions: II, effect of various abrasions and the universal fatigue curve, J. Amer. Ceram. Soc. 42 (1959) 582-592; R. E. Mould, Strength and static fatigue of abraded glass under controlled ambient conditions: IV, effect of surrounding medium, J. Amer. Ceram. Soc. 44 (1961) 481-491; S. M. Wiederhorn, Crack growth and static fatigue, Journal of Non-Crystalline Solids, 19 (1975) 169-181) an empirical equation for the time-dependent strength of the dosage forms, σ_{f}(t), may be written as:

$$\frac{{σ}_{f} ( t )}{{σ}_{f , 0}} = - a ( t - {t}_{exp} ) + 1 \qquad ( 13 )$$

where a is a constant.

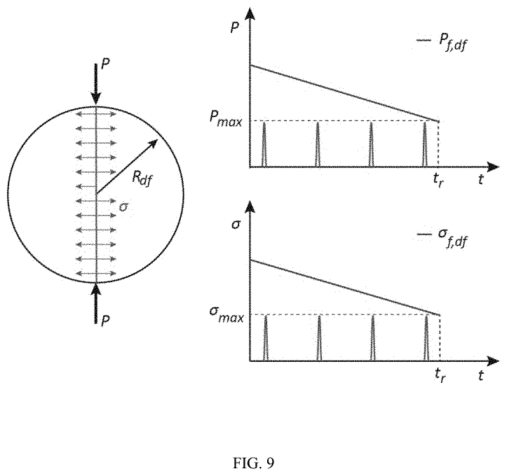

Thus, as shown schematically in FIG. 9, the strength of the expanded dosage form may decrease with time due to static fatigue. The dosage form may fracture as soon as the fracture strength is smaller the maximum stress, σₘₐₓ, applied due to the contracting stomach walls.

Substituting the maximum stress, σₘₐₓ, for the fracture stress, σ_{f}, and the gastric residence time, tᵣ, for the time, t, in Eq. (13) gives:

$$\frac{{σ}_{max}}{{σ}_{f , 0}} = - a ( {t}_{r} - {t}_{exp} ) + 1 \qquad ( 14 )$$

The fracture strength of the expanded dosage forms at time t=tₑₓₚ, may be written as:

σ_{f,0}=C₈σ_{f}φ_{c}^{m} (15)

where C₈ and m are constants, and σ_{f} the fracture strength of the physiological fluid-soaked coating.

Substituting Eq. (15) in Eq. (14) and rearranging gives:

$${t}_{r} - {t}_{exp} = \frac{1}{a} ( 1 - \frac{{σ}_{max}}{{σ}_{f} {C}_{8} {φ}_{c}^{m}} ) \qquad ( 16 )$$

Substituting the non-limiting parameters a=0.027/h, σₘₐₓ=0.0195 MPa, σ_{f}=1.8 MPa, C₈=0.93, tₑₓₚ=6 h, m=1.19, and φ_{c}=0.025-0.068 in Eq. (16), tᵣ=8-39 h.

Thus, by adjusting the nominal volume fraction of the strengthening coating, the gastric residence time of the dosage form can be controlled within about 10-40 hours and beyond.

More models for estimating the gastric residence time of dosage forms obvious to a person of ordinary skill in the art are all within the spirit and scope of this disclosure.

### (d2) Dosage Form Disintegration Due to Dynamic Fatigue

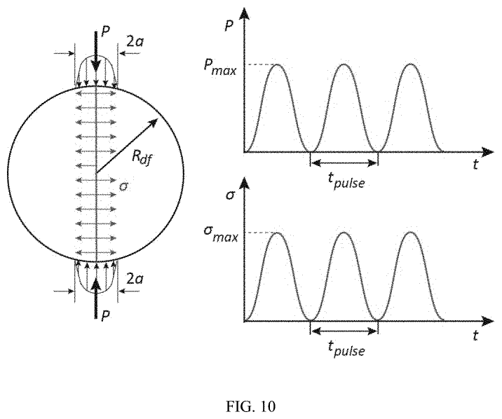

In the second model, the dosage forms is exposed to cyclic loads imposed by the contracting stomach walls, as shown in FIG. 10. It eventually fractures due to dynamic fatigue failure.

For estimating the gastric residence time due to such dynamic fatigue failure, it is assumed that the maximum load intensity Pₘₐₓ, and maximum stress, σₘₐₓ, applied by the contracting stomach walls are time-invariant. In analogy with Basquin's equation (for further details, see, e.g., O. H. Basquin, The exponential law of endurance tests. Proc. Am. Soc. Test Mater. 10, 1910, 625-630; J. C. Grosskreutz, Strengthening and fracture in fatigue (approaches for achieving high fatigue strength), Metallurgical Transactions 3, 1972, 1255-1262), a power function for the fatigue life of the dosage form may be proposed as:

σₘₐₓ=σ_{f,df}N_{f}^{b} (17)

where σ_{f,df} is the tensile strength of the dosage form, N_{f} the number of cycles to failure, and b a constant.

Rearranging Eq. (17) gives:

$${N}_{f} = {( \frac{{σ}_{max}}{{σ}_{f , df}} )}^{1 / b} \qquad ( 18 )$$

The gastric residence time, tᵣ=N_{f}×tₚᵤₗₛₑ, where tₚᵤₗₛₑ is the period of a compression cycle by the stomach walls. Substituting this term in Eq. (18) and rearranging gives:

$${t}_{r} = {{t}_{pulse} ( \frac{{σ}_{max}}{{σ}_{f , df}} )}^{1 / b} \qquad ( 19 )$$

Combining Eq. (19) with Eq. (15),

$${t}_{r} = {{t}_{pulse} ( \frac{{σ}_{max}}{{σ}_{f} {C}_{8} {φ}_{c}^{m}} )}^{1 / b} \qquad ( 20 )$$

and combining Eq. (20) with Eq. (16),

$$\begin{matrix}{t}_{r} = {{t}_{pulse} ( \frac{{P}_{max}}{π {R}_{f} {σ}_{f} {C}_{8} {φ}_{c}^{3 / 2}} )}^{1 / b} \\ or & ( 21 a ) \\ {t}_{r} = {{t}_{pulse} ( \frac{π {R}_{f} {σ}_{f} {C}_{8} {φ}_{c}^{3 / 2}}{{P}_{max}} )}^{- 1 / b} & ( 21 b )\end{matrix}$$

Substituting the non-limiting parameters (tₚᵤₗₛₑ=55 s, Pₘₐₓ=1.25 N/mm, R_{df}=11.5 mm, σ_{f}=1.8 MPa, C₈=0.65, φ_{c}=0.14, and b=−0.0692 in Eq. (21b), the gastric residence time, tᵣ=31 hours.

It may be noted again that all the above calculations are approximate and for illustrative purposes only. None of the numbers should be considered as exact. More models for estimating the gastric residence time of dosage forms obvious to a person of ordinary skill in the art are all within the spirit and scope of this disclosure.

### Embodiments of the Dosage Form

In view of the theoretical models and non-limiting examples above, which are suggestive and approximate rather than exact, and other considerations, the dosage forms disclosed herein may further comprise the following embodiments.

### (a) Outer Geometry of Drug-Containing Solid and Surface Layer-Encapsulated Three Dimensional Structural Framework of Elements

Because the dosage form (either in the solid or in the expanding or expanded state) should generally have a length greater the diameter of the pylorus to prevent its premature passage into the intestines, and the maximum expansion and the expansion rate may be limited, a greater length of a drug-containing solid or three dimensional structural framework of one or more elements can be preferred. In some embodiments, therefore, the average length, and/or the average width, and/or average thickness of a drug-containing solid (e.g., a three dimensional structural framework of one or more elements, an outer surface of a three dimensional structural framework of one or more elements, etc.) is/are greater than 1 mm. This includes, but is not limited to an average length, and/or average width, and/or average thickness of a drug-containing solid (e.g., a three dimensional structural framework of one or more elements, an outer surface of a three dimensional structural framework of one or more elements, etc.) greater than 1.5 mm, or greater than 2 mm, or greater than 3 mm, or greater than 4 mm, or greater than 5 mm, or greater than 6 mm.

To assure that the dosage form is swallowable by a human or animal subject, however, the length, width, or thickness of the dosage form should also not be too large. Thus, in some embodiments, the average length, and/or the average width, and/or average thickness of a drug-containing solid (e.g., a three dimensional structural framework of one or more elements, an outer surface of a three dimensional structural framework of one or more elements, etc.) is/are in ranges 2 mm-30 mm. This includes, but is not limited to average length, and/or the average width, and/or average thickness of a drug-containing solid (e.g., a three dimensional structural framework of one or more elements, an outer surface of a three dimensional structural framework of one or more elements) in the ranges 2 mm-25 mm, 5 mm-20 mm, 5 mm-18 mm, 6 mm-20 mm, 7 mm-20 mm, 7 mm-19 mm, 7 mm-18 mm, 7 mm-17 mm, 7 mm-16 mm, 8 mm-20 mm, 8 mm-18 mm, 8 mm-16 mm, 8 mm-15 mm, or 8 mm-14 mm. In the invention herein, “the length” is usually referred to as a measure of distance in direction of the longest distance, the thickness is usually referred to a measure of distance in direction of the shortest distance, and the width is smaller than the length but greater than the thickness. Moreover, in some embodiments the direction of the “width” may be perpendicular to the direction of the length and/or to the direction of the thickness.

In some embodiments, moreover, a width perpendicular to the direction of the longest dimension of the dosage form or drug-containing solid herein is greater than 6 mm. This includes, but is not limited to a width perpendicular to the direction of the longest dimension of the dosage form or drug-containing solid greater than 7 mm, or greater than 8 mm, or greater than 9 mm, or in the ranges 6 mm-18 mm, 6 mm-16 mm, 6 mm-15 mm, 7 mm-18 mm, 7 mm-16 mm, 7 mm-15 mm, or 8 mm-18 mm, 8 mm-16 mm, or 8 mm-15 mm.

The dosage forms or drug-containing solids or three dimensional structural frameworks herein can have any common or uncommon outer shape of a drug-containing solid (e.g., a tablet, capsule, etc.). For non-limiting examples of common tablet shapes, see, e.g., K. Alexander, Dosage forms and their routes of Administration, in M. Hacker, W. Messer, and K. Bachmann, Pharmacology: Principles and Practice, Academic Press, 2009. Any other outer geometries, outer shapes, outer surfaces, or dimensions of dosage forms, drug-containing solids, or three dimensional structural frameworks of elements obvious to a person of ordinary skill in the art are all within the spirit and scope of this invention.

### (b) Surface Composition of Encapsulating Surface Layer

In some embodiments, for enabling rapid percolation of dissolution fluid into the interior of the dosage form structure (e.g., into free space or interconnected free space of the drug-containing solid), the surface composition of at least an encapsulating surface layer is hydrophilic. Such embodiments include, but are not limited to embodiments where the surface composition of a coating of an encapsulating surface layer and/or the surface layer of a segment comprising an encapsulating surface layer, etc. is hydrophilic. In this disclosure, a surface or surface composition is hydrophilic, also referred to as “wettable by a physiological fluid”, if the contact angle of a droplet of physiological fluid on said surface in air is no more than 90 degrees. This includes, but is not limited to a contact angle of a droplet of said fluid on said solid surface in air no more than 80 degrees, or no more than 70 degrees, or no more than 60 degrees, or no more than 50 degrees, or no more than 40 degrees, or no more than 30 degrees. It may be noted that in some embodiments the contact angle may not be stationary. In this case, a solid surface may be understood “hydrophilic” if the contact angle of a droplet of physiological fluid on said solid surface in air is no more than 90 degrees (including but not limiting to no more than 80 degrees, or no more than 70 degrees, or no more than 60 degrees, or no more than 50 degrees, or no more than 40 degrees) at least 20-360 seconds after the droplet has been deposited on said surface. A non-limiting schematic of a droplet on a surface is presented in U.S. application Ser. No. 15/482,776 titled “Fibrous dosage form”.

Generally, the percolation rate of physiological fluid into free space or interconnected free space is increased if the contact angle between said fluid and the surface of the three dimensional structural framework of one or more elements is decreased. Rapid percolation of physiological fluid into free space or interconnected free space is desirable for fast expansion of a drug-containing solid or dosage form.

Any other surface compositions or coatings of an encapsulating surface layer, or of surface layer-encapsulated elements or three dimensional structural frameworks, that would be obvious to a person of ordinary skill in the art are all included in this invention.

### (c) Microstructure of Solid Core and Three Dimensional Structural Framework of Elements or Fibers

As shown later in the non-limiting experimental examples, a non-limiting method or way of manufacturing dosage forms as disclosed herein includes dip-coating a solid core with a surface-encapsulating coating. Thus, if the solid core comprises a framework of elements or fibers, to assure that the coating substantially encapsulates said framework, in some embodiments a dip-coating solution (e.g., a solution comprising at least a coating substance and a solvent) used for manufacture of dosage forms as disclosed herein should percolate into the interior of the outer volume of a solid core (e.g., into one or more element-free spaces or into one or more fiber-free spaces surrounding a three dimensional structural framework of elements or fibers). Therefore, after immersion of a solid core into a dip-coating solution, the outer volume of said solid core may comprise at least a continuous channel of element-free space or fiber-free space having at least one, and preferably at least two openings in contact with said solution. The more such channels exist with at least one, and preferably at least two ends in contact with said dip-coating solution, the more uniformly may the outer volume of said core structure be percolated. Also, the longer, more connected, and more uniformly distributed such channels may be in the outer volume of said solid core, the more uniformly may said outer volume be percolated. Uniform percolation generally is desirable in the invention herein.

Thus, in the invention herein a plurality of adjacent element-free or fiber-free spaces may combine to define one or more interconnected element-free or fiber-free spaces (e.g., element-free or fiber-free spaces that are “contiguous” or “in direct contact” or “merged” or “without any wall in between”) that may extend over a length at least half the thickness of the outer volume of a solid core or the outer volume of a three-dimensional structural framework of elements or fibers. This includes, but is not limited to a plurality of adjacent element-free or fiber-free spaces combining to define one or more interconnected element-free or fiber-free spaces that extend over a length at least two thirds the thickness of the outer volume of a solid core, or over a length at least equal to the thickness of the outer volume of a solid core, or over a length at least equal to the side length of the outer volume of a solid core, or over a length and width at least equal to half the thickness of the outer volume of a solid core, or over a length and width at least equal to the thickness of the outer volume of a solid core, or over a length, width, and thickness at least equal to half the thickness of the outer volume of a solid core, or over the entire length, width, and thickness of the outer volume of a solid core.

Also, in some embodiments an interconnected element-free or fiber-free space comprises or occupies at least 30 percent (e.g., at least 40 percent, or at least 50 percent, or at least 60 percent, or at least 70 percent, or at least 80 percent, or 100 percent) of the element-free or fiber-free space of the outer volume of a solid core (e.g., at least 30 percent, or at least 40 percent, or at least 50 percent, or at least 60 percent, or at least 70 percent, or at least 80 percent, or 100 percent of the element-free or fiber-free space of an outer volume of a solid core are part of the same interconnected element-free or fiber-free space).

In preferred embodiments, all element-free or fiber-free spaces are interconnected forming a continuous, single open space. In the invention herein, if all element-free or fiber-free spaces of an outer volume of a solid core or drug-containing solid are interconnected, the element-free or fiber-free space of said outer volume of said solid core or said drug-containing solid is also referred to as “contiguous”. In the outer volume of a solid core with contiguous element-free or fiber-free space, no walls (e.g., walls comprising the three dimensional structural framework of elements) must be ruptured to obtain an interconnected cluster of element-free or fiber-free space (e.g., an open channel of element-free or fiber-free space) from the outer surface of the solid core to a point (or to any point) in the free element-free or fiber-free space within the outer volume of the solid core. The entire element-free or fiber-free space or essentially all element-free or fiber-free spaces is/are accessible from (e.g., connected to) the outer surface of the solid core.

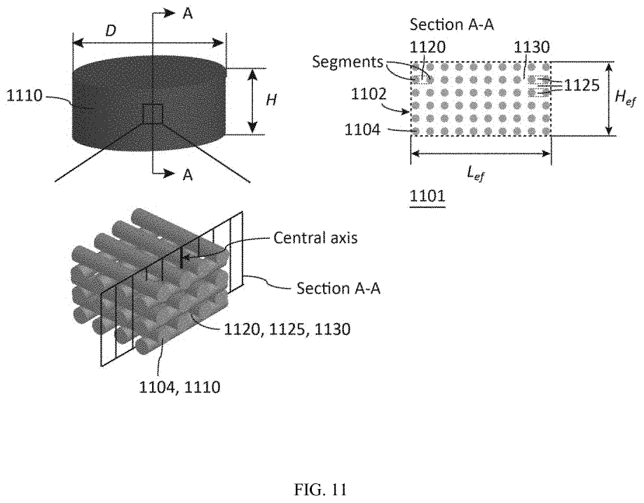

FIG. 11 schematically illustrates a non-limiting solid core comprising an outer surface and an internal, three dimensional structural framework 1104 of a plurality of criss-crossed stacked layers of fibrous structural elements 1110. Said framework 1104 is contiguous with and terminates at said outer surface 1102. The fibrous structural elements 1110 further have segments spaced apart from adjoining segments, thereby defining element-free or fiber-free spaces 1120. A plurality of adjacent element-free or fiber-free spaces 1125 combine to define one or more interconnected element-free or fiber-free spaces 1130.

As shown in the non-limiting schematic of section A-A, at least one interconnected element-free or fiber-free space 1130 extends over the entire length and thickness of the outer volume of the three dimensional structural framework 1104. In other words, the length, Lₑf, over which the interconnected element-free or fiber-free space 1130 extends is the same or about the same as the length of the outer volume of the three dimensional structural framework 1104; the thickness, Hₑf, over which the interconnected element-free or fiber-free space 1130 extends is the same or about the same as the thickness, H, of the outer volume of the three dimensional structural framework 1104. It may be noted that the term “section” is understood herein as “plane” or “surface”. Thus a “section” is not a “projection” or “projected view”.

Moreover, in the non-limiting example of FIG. 11 the microstructure is rotationally symmetric. If the plane or section A-A is rotated by 90 degrees about the central axis the microstructure (e.g., the microstructural details) is/are the same. Thus, the interconnected element-free or fiber-free space 1130 also extends over the entire width of the outer volume of the three dimensional structural framework 1104. In other words, the width over which the interconnected element-free or fiber-free space 1130 extends is the same or about the same as the width of the outer volume of the three dimensional structural framework 1104.

Furthermore, in the non-limiting microstructure of FIG. 11, as shown in section A-A the interconnected element-free or fiber-free space 1130 (or element-free or fiber-free space 1120 or element-free or fiber-free spaces 1125) is/are contiguous. No walls (e.g., walls comprising the three dimensional structural framework 1304 of elements) must be ruptured to obtain an interconnected cluster of element-free or fiber-free spaces (e.g., an open channel of element-free or fiber-free space) from the outer surface 1102 of the three dimensional structural framework 1104 to a point (or to any point or position) in the element-free or fiber-free space 1120, 1125, 1130. Also, no walls (e.g., walls comprising the three dimensional structural framework 1104 of elements) must be ruptured to obtain an interconnected cluster of element-free or fiber-free space (e.g., an open channel of element-free or fiber-free space) from any point or position within the element-free or fiber-free space 1120, 1125, 1130 to any other point or position in the element-free or fiber-free space 1120, 1125, 1130. The entire element-free or fiber-free space 1120, 1125, 1130 is accessible from the outer surface 1102 of the three dimensional structural framework of fibers 1104. In addition, the entire element-free or fiber-free space 1120, 1125, 1130 is accessible from any point, location, or position within the element-free or fiber-free space 1120, 1125, 1130.

Additionally, the structure shown in FIG. 11 comprises fibers in a layer that are aligned unidirectionally (e.g., parallel). The fibers in the layers above and below said layer are aligned unidirectionally, too, and are aligned orthogonally to said layer (e.g., the fibers in the the layers above and below said layer are aligned orthogonally to the fibers in said layer, and vice versa). The fibers in the layers above and below said layer further touch or “merge with” fibers in said layer at inter-fiber contacts.

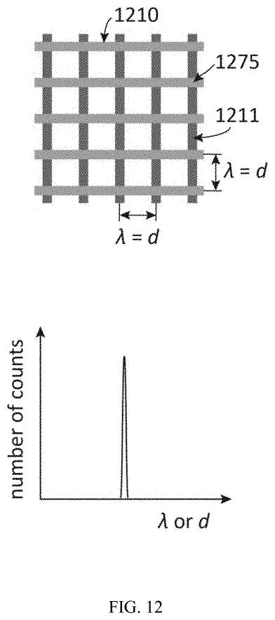

Several microstructural features can be defined to further characterize such structures. By way of example but not by way of limitation, as shown in FIG. 12, the structural framework 1210 may be considered a network comprising nodes or vertices at the inter-fiber contacts 1275 and edges 1211 defined by the fiber segments of length, λ, between adjacent nodes or vertices 1275.

FIG. 12 also shows a histogram of the length, λ, of fiber segments between adjacent point contacts 1275. The λ values in this non-limiting example are distributed in a very narrow window or zone around the average, λ_{avg}. Thus, the standard deviation of the values is very small; λ is precisely controlled; the structure is repeatable, regular, deterministic, and ordered. (Generally, structural elements are understood as “repeatably arranged” if such structural features as spacing between elements, orientation of elements, etc. is/are controlled. A structural feature is referred to as “controlled” if a standard deviation of said feature across a three dimensional structural framework (or across multiple three dimensional structural frameworks of multiple dosage forms, etc.) is smaller (or much smaller) than that in a random structure with randomly arranged elements.)

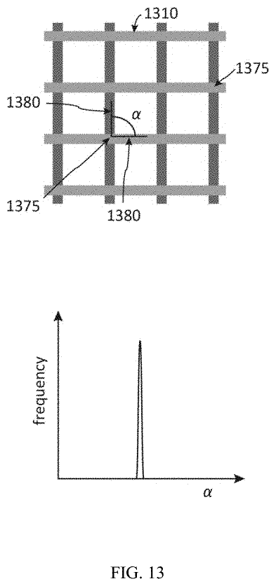

Similarly, as shown in FIG. 13, at the inter-fiber contacts 1375 the two tangents of two contacting fibers or fiber segments 1380, may form an angle, α. FIG. 13 also shows a histogram of the contact angle, α, across the three dimensional structural framework 1310. Also the α values in this non-limiting example are distributed in a very narrow window or zone around the average, α_{avg}. Thus, the standard deviation of the a values is very small; α is precisely controlled; the structure is repeatable, regular, deterministic, and ordered.

More examples of fibrous structures according to the invention herein would be obvious to a person of ordinary skill in the art. All of them are within the scope of this disclosure. Furthermore, many of the above features and characteristics may also apply to (e.g., the features or characteristics may be similar to the features or characteristics of) three-dimensional structural frameworks of stacked layers of sheets, or stacked layers of beads (or particles), as shown, for example, in the co-pending International Application No. PCT/US2019/052030 filed on Sep. 19, 2019, and titled “Dosage form comprising structured solid-solution framework of sparingly-soluble drug and method for manufacture thereof”. Such features or characteristics are obvious to a person of ordinary skill in the art who is given all information disclosed in this specification. Application of such features or characteristics to three-dimensional structural frameworks of stacked layers of beads (or particles), or stacked layers sheets (or two-dimensional elements), is included in the invention herein.

Any more microstructures of solid cores and three dimensional structural frameworks of elements or fibers would be obvious to a person of ordinary skill in the art. All of them are included in this invention.

### (d) Element-Free or Fiber-Free Spacing

Typically, moreover, for a coating solution to percolate into the interior of the structure the channel size or diameter (e.g., channel width, or pore size, or free spacing, or effective free spacing) between elements, fibers, or segments must be on the micro- or macro-scale. Thus, in some embodiments, the element-free spacing, λ_{fe}, between elements (e.g., fibers) or segments across one or more connected free element-free spaces (e.g., the channel size or channel diameter) is greater than 1 μm. This includes, but is not limited to λ_{fe} greater than 1.25 μm, or greater than 1.5 μm, or greater than 1.75 μm, or greater than 2 μm, or greater than 5 μm, or greater than 7 μm, or greater than 10 μm, or greater than 15 μm, or greater than 20 μm, or greater than 25 μm, or greater than 30 μm, or greater than 40 μm, or greater than 50 μm.

Because the dosage form volume is generally limited, however, the drug and excipient masses that can be loaded in the dosage form may be too small if the element-free spacing is too large. Thus, in some embodiments, the element-free spacing across an interconnected element-free space may be in the ranges 1 μm-5 mm, 1 μm-3 mm, 1.25 μm-5 mm, 1.5 μm-5 mm, 1.5 μm-3 mm, 5 μm-2.5 mm, 10 μm-2 mm, 10 μm-4 mm, 5 μm-4 mm, 10 μm-3 mm, 15 μm-3 mm, 20 μm-3 mm, 30 μm-4 mm, 40 μm-4 mm, or 50 μm-4 mm.

In some embodiments, moreover, the element-free spacing between segments or elements across the one or more element-free spaces (e.g., across all free spaces of the dosage form) is in the range 1 μm-3 mm. This includes, but is not limited to an element-free spacing between segments or elements across the one or more element-free spaces in the ranges 1 μm-2.5 mm, or 1 μm-2 mm, or 2 μm-3 mm, or 2 μm-2.5 mm, or 5 μm-3 mm, or 5 μm-2.5 mm, or 10 μm-3 mm, or 10 μm-2.5 mm, or 15 μm-3 mm, or 15 μm-2.5 mm, or 20 μm-3 mm, or 20 μm-2.5 mm. The element-free spacing may be determined experimentally from microstructural images (e.g., scanning electron micrographs, micro computed tomography scans, and so on) of the drug-containing solid. Non-limiting examples describing and illustrating how an element-free spacing may be determined from microstructural images are described and illustrated in the U.S. application Ser. No. 15/482,776 titled “Fibrous dosage form”.

It may be noted, moreover, that in some embodiments herein the element-free spacing between elements or segments across a three dimensional structural framework or across one or more interconnected element-free spaces is precisely controlled.

Any more details of element-free or fiber-free spacings would be obvious to a person of ordinary skill in the art. All of them are included in this invention.

### (e) Microstructure of Surface Layer-Encapsulated Solid Core and Surface Layer-Encapsulated Three Dimensional Structural Framework of Elements

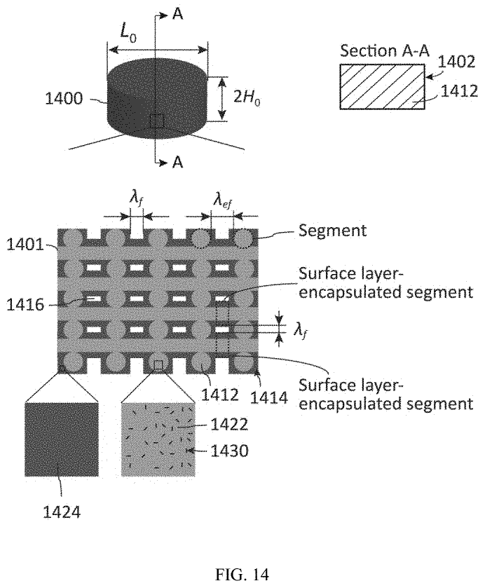

FIG. 14 presents a non-limiting dosage form comprising a drug-containing solid having a fluid-absorptive solid core 1412 and a mechanically strengthening, semi-permeable surface layer 1414. The fluid-absorptive solid core 1412 comprises a three-dimensional structural framework of structural elements 1412. The elements 1412 comprise segments separated and spaced apart from adjoining segments by element-free or spacings, λ_{ef}, defining one or more element-free spaces 1414, 1416 in the drug-containing solid 1401. The elements 1412 are further substantially encapsulated (e.g., substantially coated, substantially surrounded, etc.) by said mechanically strengthening, semi-permeable surface layer 1414. The semi-permeable surface layer 1414 comprises at least a mechanically strengthening second excipient 1424. The surface layer-encapsulated elements 1412, 1414 (e.g., the elements 1412 and the surface layer 1414 combined) further comprise surface layer-encapsulated segments spaced apart from adjoining surface layer-encapsulated segments by free spacings, λ_{f}, thereby defining one or more free spaces 1416 in the drug-containing solid 1401.

In some embodiments, upon exposure of the dosage form, drug-containing solid, or outer volume of the three dimensional structural framework to a physiological or dissolution fluid, said physiological or dissolution fluid may percolate into the interior of the structure (e.g., into one or more free spaces) if the drug-containing solid comprises at least a continuous channel of free space having at least one, and preferably at least two openings in contact with said fluid. The more such channels exist with at least one, and preferably at least two ends in contact with said fluid, the more uniformly may the structure be percolated. Uniform percolation of a dosage form, drug-containing solid, or outer volume of a structural framework by physiological or dissolution fluid is desirable, for example, to achieve rapid expansion of said dosage form, drug-containing solid, or outer volume of said structural framework after exposure to said physiological fluid.

Thus, in the invention herein a plurality of adjacent free spaces may combine to define one or more interconnected free spaces (e.g., free spaces that are “contiguous” or “in direct contact” or “merged” or “without any wall in between”). Said interconnected free spaces may extend over a length at least half the thickness of the outer volume of a solid core or the outer volume of a three-dimensional structural framework of elements. This includes, but is not limited to a plurality of adjacent free spaces combining to define one or more interconnected free spaces that extend over a length at least two thirds the thickness of the outer volume of a solid core, or over a length at least equal to the thickness of the outer volume of a solid core, and so on.

Any more microstructures of surface layer-encapsulated solid core and surface layer-encapsulated three dimensional structural framework of elements would be obvious to a person of ordinary skill in the art. All of them are included in this invention.

### (f) Free Spacing Between Surface Layer-Encapsulated Elements

Typically, moreover, for dissolution fluid to percolate into the interior of the structure the channel size or diameter (e.g., channel width, or pore size, or free spacing, or effective free spacing) between elements or segments must be on the micro- or macro-scale. Thus, in some embodiments, the free spacing, λ_{f}, between elements (e.g., fibers) or segments across one or more connected free spaces (e.g., the channel size or channel diameter) is greater than 1 μm. This includes, but is not limited to λ_{f} greater than 1.25 μm, or greater than 1.5 μm, or greater than 1.75 μm, or greater than 2 μm, or greater than 5 μm, or greater than 7 μm, or greater than 10 μm, or greater than 15 μm, or greater than 20 μm, or greater than 25 μm, or greater than 30 μm, or greater than 40 μm, or greater than 50 μm.

Because the dosage form volume is generally limited, however, the drug and excipient masses that can be loaded in the dosage form may be too small if the effective free spacing is too large. Thus, in some embodiments, the free spacing across an interconnected free space may be in the ranges 1 μm-5 mm, 1 μm-3 mm, 1.25 μm-5 mm, 1.5 μm-5 mm, 1.5 μm-3 mm, 5 μm-2.5 mm, 10 μm-2 mm, 10 μm-4 mm, 5 μm-4 mm, 10 μm-3 mm, 15 μm-3 mm, 20 μm-3 mm, 30 μm-4 mm, 40 μm-4 mm, or 50 μm-4 mm.

In some embodiments, moreover, the free spacing between segments or elements across the one or more free spaces (e.g., across all free spaces of the dosage form) is in the range 1 μm-3 mm. This includes, but is not limited to a free spacing between segments or elements across the one or more free spaces in the ranges 1 μm-2.5 mm, or 1 μm-2 mm, or 2 μm-3 mm, or 2 μm-2.5 mm, or 5 μm-3 mm, or 5 μm-2.5 mm, or 10 μm-3 mm, or 10 μm-2.5 mm, or 15 μm-3 mm, or 15 μm-2.5 mm, or 20 μm-3 mm, or 20 μm-2.5 mm. The free spacing may be determined experimentally from microstructural images (e.g., scanning electron micrographs, micro computed tomography scans, and so on) of the drug-containing solid. Non-limiting examples describing and illustrating how a free spacing may be determined from microstructural images are described and illustrated in the U.S. application Ser. No. 15/482,776 titled “Fibrous dosage form”.

It may be noted, that in some embodiments herein the free spacing between elements or segments across the three dimensional structural framework or across one or more free spaces is precisely controlled.

Furthermore, it may be noted that the free spacing between elements and the surface composition of elements are generally designed to enable percolation of physiological, body, or dissolution fluid into the dosage form structure upon immersion of the dosage form in said fluid.

Any more details of free spacings would be obvious to a person of ordinary skill in the art. All of them are included in this invention.

### (g) Composition of Free Space

Generally, one or more free spaces (e.g., one or more interconnected free spaces) are filled with a matter that is removable by a physiological fluid under physiological conditions. Such matter that is removable by a physiological fluid under physiological conditions can, for example, be a gas which escapes a free space upon percolation of said free space by said physiological fluid. Such matter that is removable by a physiological fluid under physiological conditions can, however, also be a solid that is highly soluble in said physiological fluid, and thus dissolves rapidly upon contact with or immersion in said physiological fluid.

In preferred embodiments, a biocompatible gas that may fill free space includes air. Other non-limiting examples of biocompatible gases that may fill free space include nitrogen, CO₂, argon, oxygen, and nitric oxide, among others.

Non-limiting examples of solids that are removed or dissolved after contact with physiological/body fluid include sugars or polyols, such as Sucrose, Fructose, Galactose, Lactose, Maltose, Glucose, Maltodextrin, Mannitol, Maltitol, Isomalt, Lactitol, Xylitol, Sorbitol, among others. Other examples of solids include polymers, such as polyethylene glycol, polyvinyl pyrrolidone, polyvinyl alcohol, among others. Typically, a solid material should have a solubility in physiological/body fluid (e.g., an aqueous physiological or body fluid) under physiological conditions greater than about 25 g/l to be removed or dissolved rapidly after contact with dissolution medium. This includes, but is not limited to a solubility greater than 50 g/l, or greater than 75 g/l, or greater than 100 g/l, or greater than 150 g/l. The diffusivity of the solid material (as dissolved molecule in physiological/body fluid under physiological conditions) should typically be greater than about 4×10⁻¹² m²/s if the solid material must be dissolved rapidly after contact with dissolution medium. This includes, but is not limited to a diffusivity in physiological fluid under physiological conditions greater than 6×10⁻¹² m²/s, or greater than 8×10⁻¹² m²/s, or greater than 1×10⁻¹ m²/s, or greater than 2×10⁻¹ m²/s, or greater than 5×10⁻¹ m²/s.

In some embodiments, moreover, a solid that may fill free space has a molecular weight (e.g., average molecular weight, such as number average molecular weight or weight average molecular weight) no greater than about 80 kg/mol. This includes, but is not limited to a molecular weight (e.g., average molecular weight, such as number average molecular weight or weight average molecular weight) no greater than 70 kg/mol, or no greater than 60 kg/mol, or no greater than 50 kg/mol, or no greater than 45 kg/mol, or no greater than 40 kg/mol, or no greater than 35 kg/mol.

Further compositions of free space obvious to a person of ordinary skill in the art who is given all information of this specification are all included in this invention.

### (h) Geometry of Elements

After percolation of free space or one or more interconnected free spaces, dissolution fluid or physiological fluid may surround one or more elements (e.g., fibers) or segments thereof. For achieving a large specific surface area (i.e., a large surface area-to-volume ratio) of elements in contact with dissolution fluid, in some embodiments the one or more elements (e.g., fibers) have an average thickness, h₀, no greater than 2.5 mm. This includes, but is not limited to h₀ no greater than 2 mm, or no greater than 1.75 mm, or no greater than 1.5 mm, or no greater than 1.25 mm, or no greater than 1 mm, or no greater than 750 μm.

It may be noted, however, that if the elements are very thin and tightly packed, the spacing and free spacing between the elements can be so small that the rate at which dissolution fluid percolates or flows into the free space is limited. Furthermore, dosage forms with very thin elements may be difficult to manufacture by, for example, 3D-micro-patterning. Thus, in some embodiments the one or more elements (e.g., fibers) have an average thickness, h₀, in the range of 5 μm-2.5 mm. This includes, but is not limited to average thickness, h₀, of one or more elements (e.g., fibers) in the ranges 10 μm-2 mm, 20 μm-2 mm, 25 μm-2 mm, 30 μm-2 mm, 20 μm-1.5 mm, 25 μm-1.5 mm, 25 μm-1.25 mm, 25 μm-1 mm, 30 μm-1.5 mm, 30 μm-1.25 mm, 30 μm-1 μm, 40 μm-1.5 mm, or 50 μm-1.25 mm.

In some embodiments, moreover, the average thickness of the one or more elements (e.g., fibers) comprising or composing (e.g., producing, making up, etc.) the three dimensional structural network (e.g., the average thickness of the elements in the three dimensional structural network) is precisely controlled.

The element or fiber thickness, h, may be considered the smallest dimension of an element (i.e., h≤w and h≤l, where h, w and l are the thickness, width and length of the element, respectively). The average element or fiber thickness, h₀, is the average of the element or fiber thickness along the length of the one or more elements or fibers. A non-limiting example illustrating how an average fiber thickness may be derived is presented in U.S. application Ser. No. 15/482,776 titled “Fibrous dosage form”.

In some embodiments, at least one outer surface of one or more elements (e.g., the outer surface or one or more fibers or the outer surface of a fiber segment) comprises a coating. Said coating may cover part of or the entire outer surface of one or more elements or a segment thereof. Said coating may further have a composition that is different from the composition of one or more elements or a segment thereof. The coating may be a solid, and may or may not comprise or contain a drug.

Any more element or fiber geometries would be obvious to a person of ordinary skill in the art. All of them are included in this invention.

### (i) Micro- and Nano-Structure of Solid Core and Encapsulating Surface Layer

Generally, in the invention herein, a solid core (e.g., a fluid-absorptive solid core, element, fiber, and so on) comprises at least a physiological fluid-absorptive excipient. In preferred embodiments, said solid core (e.g., said fluid-absorptive solid core, element, fiber, and so on) further comprises at least a drug. In preferred embodiments, moreover, said drug and said fluid-absorptive excipient may be mixed together forming a mixture of drug and fluid-absorptive excipient. Within said mixture, the drug may generally be molecularly distributed or molecularly dissolved in one or more absorptive excipients (e.g., drug molecules may be mixed with absorptive excipient), or it may be dispersed or distributed as drug particles in a fluid-absorptive excipient matrix, or it may be combined with one or more fluid-absorptive excipients by other means.

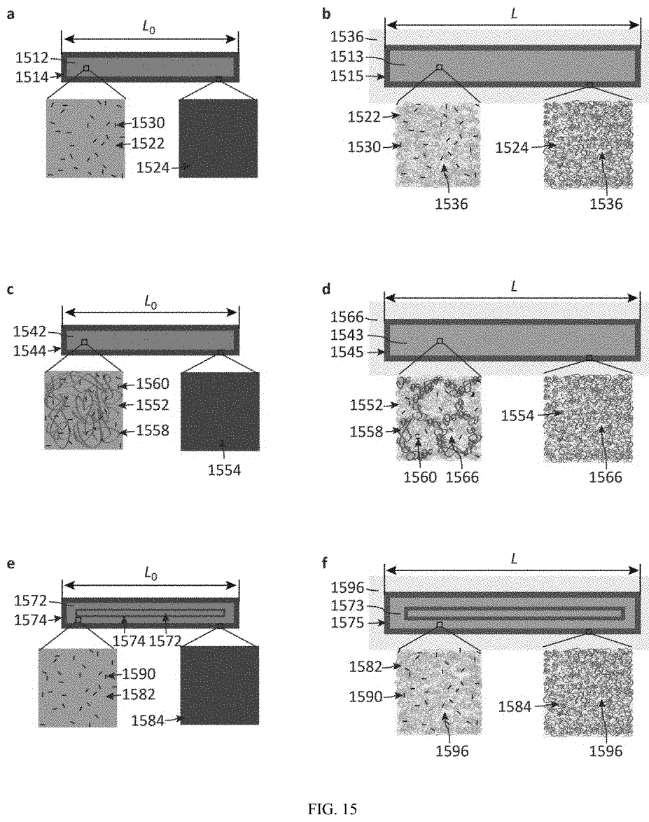

FIG. 15a presents a non-limiting example of a solid core 1512 (e.g., a fluid-absorptive solid core, structural element, fiber, etc.) substantially encapsulated by a mechanically strengthening, semi-permeable surface layer 1514 (e.g. a coating, etc.). The solid core 1512 comprises a mixture of at least a fluid-absorptive first excipient 1522 (e.g., one or more fluid-absorptive first excipients) and at least a drug 1530. Said fluid-absorptive first excipient 1522 may comprise at least a polymer. The mechanically strengthening, semi-permeable surface layer comprises at least a mechanically strengthening second excipient 1524 (e.g., one or more mechanically strengthening second excipinents). Said mechanically strengthening second excipient 1524 may comprise at least a polymeric constituent (e.g., at least a polymer).

As shown schematically in the non-limiting FIG. 15b, upon exposure to physiological fluid 1536, such as saliva, gastric fluid, a fluid that resembles a physiological fluid, and so on, the surface layer 1514 encapsulated solid core 1512 expands primarily with fluid 1530 absorption, thereby transitioning to a viscous or semi-solid mass 1513. Additionally, the mechanically strenghtening, semi-permeable surface layer 1514 forms a semi-permeable, viscoelastic membrane 1515. The semi-permeable, viscoelastic membrane expands 1514, 1515 due to an internal pressure in the core 1512, 1513 generated by osmotic flow of fluid 1536 into said core 1512, 1513. As a result, upon exposure to said physiological fluid 1536, the surface-encapsulated solid core 1512, 1514 (e.g., the solid core and surface layer combined) forms an expanded, viscoelastic composite mass 1510 having a length (e.g., L) greater than 1.2 times (e.g., greater than 1.3 times) its length prior to exposure to said physiological fluid 1536 (e.g., L₀).

In some embodiments, a fluid-absorptive solid core (e.g., an element, fiber, etc.) may further comprise a third excipient. By way of example but not by way of limitation, said third excipient can be a mechanically strengthening excipient, a filler, and so on.

FIG. 15c presents another non-limiting example of a solid core 1542 (e.g., a fluid-absorptive solid core, structural element, fiber, etc.) substantially encapsulated by a mechanically strengthening, semi-permeable surface layer 1544. The solid core 1542 (e.g., the fluid-absorptive solid core, structural element, fiber, etc.) comprises a mixture (e.g., a solid solution) of drug 1560, at least a physiological fluid-absorptive first excipient 1552, and at least a mechanically strengthening third excipient 1558. Said fluid-absorptive first excipient 1552 and said mechanically strengthening third excipient 1558 may comprise at least a polymer. The mechanically strengthening, semi-permeable surface layer 1544. comprises at least a mechanically strengthening second excipient 1554. Said mechanically strengthening second excipient 1554 may comprise at least a polymeric constituent (e.g., at least a polymer).

Upon exposure to physiological fluid 1566, such as saliva, gastric fluid, a fluid that resembles a physiological fluid, and so on, the one or more mechanically strenghtening third excipients 1558 may form a fluid-permeable, semi-solid network 1558 to mechanically support the core 1542, 1543 FIG. 15d. Also, the one or more fluid-absorptive excipients 1552 may transition to a viscous mass, or a viscous solution 1543, expanding said solid core, element, fiber, etc. 1542, 1543 along at least one dimension (or in all dimensions) with absorption of said physiological fluid 1556. As a result, the surface layer 1544 encapsulated solid core 1542 expands with fluid 1556 absorption, thereby transitioning to a viscous or semi-solid mass 1543. Additionally the mechanically strenghtening, semi-permeable surface layer 1544 may form a semi-permeable, viscoelastic membrane 1545. The semi-permeable, viscoelastic membrane 1544, 1545 may expand due to an internal pressure in the core 1542, 1543 generated by osmotic flow of fluid 1556 into said core 1542, 1543. As a result, upon exposure to said physiological fluid 1556 the surface-encapsulated solid core 1542, 1544 (e.g., the solid core and surface layer combined) may form an expanded, viscoelastic composite mass 1540 having a length (e.g., L) greater than 1.2 times (e.g., greater than 1.3 times) its length prior to exposure to said physiological fluid (e.g., L₀).

Many more microstructures of drug-containing solids according to this invention could be presented. By way of example but not by way of limitation, FIG. 15e presents a non-limiting example of a drug-containing solid comprising multiple solid cores 1572 and multiple mechanically strengthening, semi-permeable surface layer 1574. The solid cores 1572 comprise a mixture of at least a fluid-absorptive first excipient 1582 (e.g., one or more fluid-absorptive first excipients) and at least a drug 1590. Said fluid-absorptive first excipient 1582 may comprise at least a polymer. The mechanically strengthening, semi-permeable surface layers comprise at least a mechanically strengthening second excipient 1584 (e.g., one or more mechanically strengthening second excipinents). Said mechanically strengthening second excipient 1584 may comprise at least a polymeric constituent (e.g., at least a polymer).

As shown schematically in the non-limiting FIG. 15f, upon exposure to physiological fluid 1596, such as saliva, gastric fluid, a fluid that resembles a physiological fluid, and so on, the surface layer 1574 encapsulated solid cores 1572 expand primarily with fluid 1596 absorption, thereby transitioning to a viscous or semi-solid mass 1573. Additionally, the mechanically strenghtening, semi-permeable surface layers 1574 form semi-permeable, viscoelastic membranes 1575. The semi-permeable, viscoelastic membranes expand 1574, 1575 due to an internal pressure in the cores 1572, 1573 generated by osmotic flow of fluid 1596 into said cores 1572, 1573. As a result, upon exposure to said physiological fluid 1596, the surface-encapsulated solid cores 1572, 1574 (e.g., the solid cores and surface layers combined) form an expanded, viscoelastic composite mass 1570 having a length (e.g., L) greater than 1.2 times (e.g., greater than 1.3 times) its length prior to exposure to said physiological fluid 1596 (e.g., L₀).

In some embodiments, the concentration of at least an absorptive excipient is substantially uniform within or through or across a solid core (e.g., one or more elements, a three dimensional structural framework of elements, etc.).

In some embodiments, moreover, a solid core (e.g., one or more elements, etc.) comprise a plurality of (e.g., two or more) segments having substantially the same weight fraction of physiological fluid-absorptive excipient distributed within the segments (e.g., the standard deviation of the weight fraction of absorptive excipient within the segments is no greater than the average value).

In some embodiments the weight fraction of absorptive polymeric excipient in at least a solid core (e.g., one or more elements, etc.) with respect to the total weight of said solid core is greater than 0.1. This includes, but is not limited to a weight fraction of absorptive polymeric excipient in a solid core with respect to the total weight of said solid core greater than 0.15, or greater than 0.2, or greater than 0.25, or greater than 0.3, or greater than 0.35, or greater than 0.4.

Similarly, in some embodiments the weight fraction of absorptive polymeric excipient in a three dimensional structural framework of one or more elements (e.g., fibers) with respect to the total weight of said framework is greater than 0.1. This includes, but is not limited to a weight fraction of absorptive, polymeric excipient in the structural framework with respect to the total weight of said framework greater than 0.15, or greater than 0.2, or greater than 0.25, or greater than 0.3, or greater than 0.35, or greater than 0.4.

In some embodiments, the volume of mechanically strengthening semi-permeable surface layer per unit volume of the dosage form or of a drug-containing solid (e.g., the volume fraction of mechanically strengthening semi-permeable surface layer in the dosage form or in a drug-containing solid) is greater than 0.005. This includes, but is not limited to a volume of mechanically strengthening semi-permeable surface layer per unit volume of the dosage form or of a drug-containing solid (e.g., the volume fraction of strength-enhancing excipient in the dosage form or in a drug-containing solid with respect to the volume of said dosage form or of said drug-containing solid) greater than 0.01, or greater than 0.015, or greater than 0.02, or greater than 0.025.

In some embodiments, the weight of mechanically strengthening semi-permeable surface layer per unit volume of the dosage form or of a drug-containing solid (e.g., the density of mechanically strengthening semi-permeable surface layer in the dosage form or in a drug-containing solid) is greater than 5 kg/m³. This includes, but is not limited to a weight of mechanically strengthening semi-permeable surface layer per unit volume of the dosage form or of a drug-containing solid (e.g., the density of mechanically strengthening semi-permeable surface layer in the dosage form or in a drug-containing solid) greater than 10 kg/m³, or greater than 15 kg/m³, or greater than 20 kg/m³.

In some embodiments of any dosage form disclosed herein, the volume or weight fraction of a mechanically strengthening semi-permeable surface layer in a drug-containing solid or dosage form may be in the range between 0.005 and 0.6. This includes, but is not limited to a volume or weight fraction of a mechanically strengthening semi-permeable surface layer in the drug-containing solid or dosage form in the range between 0.01 and 0.6, or between 0.005 and 0.55, or between 0.01 and 0.55, or between 0.005 and 0.5, or between 0.01 and 0.5, or between 0.005 and 0.45, or between 0.01 and 0.45, or between 0.005 and 0.4, or between 0.01 and 0.4.

In some embodiments, moreover, a mechanically strengthening semi-permeable surface layer may comprise a thickness greater than 1 μm. This includes, but is not limited to a mechanically strengthening semi-permeable surface layer comprising a thickness greater than 2 μm, or greater than 5 μm, or greater than 10 μm.

Any further microstructures of drug containing solids or solid cores and encapsulating surface layers would be obvious to a person of ordinary skill in the art. All of them are within the spirit and scope of this invention.

### (j) Properties and Composition of Absorptive Excipient

The drug-containing elements herein comprise at least one ore more physiological fluid-absorptive excipients. In some specific embodiments embodiments, an absorptive excipient may be mutually soluble with a relevant physiological fluid under physiological conditions, and thus “absorb” or “mix with” said physiological fluid until its concentration is uniform across said fluid. Accordingly, absorptive excipient may promote expansion and dissolution and/or disintegration of a drug-containing solid or a viscoelastic mass.

In some embodiments, moreover the effective diffusivity of physiological/body fluid in an absorptive excipient (and/or an element or a segment) is greater than 0.05×10⁻¹¹ m²/s under physiological conditions. This includes, but is not limited to an effective diffusivity of physiological/body fluid in an absorptive excipient (and/or an element or a segment) greater than 0.1×10⁻¹¹ m²/s, or greater than 0.2×10⁻¹¹ m²/s, or greater than 0.5×10⁻¹¹ m²/s, or greater than 0.75×10⁻¹¹ m²/s, or greater than 1×10−1 m²/s, or greater than 2×10⁻¹¹ m²/s, or greater than 3×10⁻¹¹ m²/s, or greater than 4×10⁻¹¹ m²/s under physiological conditions.

Alternatively, for absorptive excipients where diffusion of physiological/body fluid to the interior may or may not be Fickian, a rate of penetration may be specified. In some embodiments, the rate of penetration of a physiological/body fluid into a solid, absorptive excipient (and/or an element or a segment) is greater than an average thickness of the one or more drug-containing elements divided by 3600 seconds (i.e., h₀/3600 μm/s). In other examples without limitation, rate of penetration may be greater than h₀/1800 μm/s, greater than h₀/1200 μm/s, greater than h₀/800 μm/s, greater than h₀/600 μm/s, or greater than h₀/500 μm/s, or greater than h₀/400 μm/s, or greater than h₀/300 μm/s.

For determining the effective diffusivity (and/or the rate of penetration) of dissolution medium in a solid, absorptive excipient (and/or an element or a segment) the following procedure may be applied. An element (e.g an element or segment of the dosage form structure, or preferably an element or segment that just consists of the absorptive excipient) may be placed in a still dissolution medium at 37° C. The time t₁ for the element to break apart or deform substantially may be recorded. (By way of example but not by way of limitation, a deformation of an element may generally be considered substantial if either the length, width, or thickness of the element differs by at least 20 to 80 percent (e.g., at least 20 percent, or at least 30 percent, or at least 40 percent, or at least 50 percent, or at least 60 percent, or at least 70 percent, or at least 80 percent, etc.) from its initial value.) The effective diffusivity, D_{eff}, may then be determined according to D_{eff}=hᵢₙᵢₜ²/4t₁ where hᵢₙᵢₜ is the initial element or segment thickness (e.g., the thickness of the dry element or segment). Similarly, the rate of penetration of a physiological/body fluid into the element or segment may be equal to hᵢₙᵢₜ/2t₁. Further non-limiting examples for deriving the effective diffusivity or rate of penetration are presented in U.S. application Ser. No. 15/482,776 titled “Fibrous dosage form”.

To ensure that the drug-containing solid expands substantially, and that the integrity of the expanded viscoelastic mass is preserved for prolonged time within a physiological fluid under physiological conditions, the molecular weight of the one or more physiological fluid-absorptive excipients may be quite large. In some embodiments, therefore, the molecular weight of at least one absorptive polymeric excipient is greater than 30 kg/mol. This includes, but is not limited to a molecular weight of an absorptive polymeric excipient greater than 40 kg/mol, or greater than 50 kg/mol, or greater than 60 kg/mol, or greater than 70 kg/mol, or greater than 80 kg/mol.

To ensure that the dosage form can be processed by patterning a viscous drug-excipient paste, and for other reasons, the molecular weight of at least one absorptive excipient (or the absorptive polymeric excipient in its totality) may, however, also be limited.

By way of example but not by way of limitation, the molecular weight of at least one absorptive excipient (or the average molecular weight of the absorptive excipient in its totality) may be in the ranges 30 kg/mol-10,000,000 kg/mol, 50 kg/mol-10,000,000 kg/mol, 70 kg/mol-10,000,000 kg/mol, 80 kg/mol-10,000,000 kg/mol, 70 kg/mol-5,000,000 kg/mol, 70 kg/mol-2,000,000 kg/mol. Preferably, a physiological fluid-absorptive excipient comprises hydroxypropyl methylcellulose with a molecular weight in the range between about 50 kg/mol and 500 kg/mol (e.g., 70 kg/mol-300,000 kg/mol).

Thus, in some embodiments, at least one absorptive excipient (or the absorptive excipient in its totality) may comprise a plurality of individual chains or molecules that dissolve or disentangle upon immersion in a physiological fluid.

In some embodiments, moreover, at least one absorptive excipient has a solubility greater than 20 g/l in a relevant physiological/body fluid under physiological conditions. This includes, but is not limited to at least one absorptive excipient (or the absorptive excipient in its totality) having a solubility in a relevant physiological/body fluid under physiological conditions greater than 50 g/l, or greater than 75 g/l, or greater than 100 g/l, or greater than 150 g/l, or greater than 175 g/l, or greater than 200 g/l, or greater than 250 g/l, or greater than 300 g/l, or greater than 350 g/l. In the extreme case, absorptive excipient (e.g., at least one absorptive excipient or the absorptive excipient in its totality) is mutually soluble with a relevant physiological fluid under physiological conditions. The solubility of a material is referred to herein as the maximum amount or mass of said material that can be dissolved at equilibrium in a given volume of physiological fluid under physiological conditions divided by the volume of said fluid or of the solution formed. By way of example but not by way of limitation, the solubility of a solute in a solvent may be determined by optical methods.

Preferably, moreover, at least one absorptive polymeric excipient (or the absorptive polymeric excipient in its totality) comprises an amorphous molecular structure (e.g., an amorphous arrangement of molecules, or an arrangement of molecules without long-range order) in the solid state. A non-limiting method for determining the molecular structure of a solid (e.g., distinguishing amorphous molecular structure from crystalline molecular structure, etc.) is Differential scanning calorimetry.

Non-limiting examples of excipients that satisfy some or all the requirements of an absorptive polymeric excipient include but are not limited to hydroxypropyl methylcellulose, hydroxyethyl cellulose, polyvinyl alcohol, polyvinylpyrrolidone, hydroxypropyl methylcellulose acetate succinate, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), vinylpyrrolidone-vinyl acetate copolymer, among others.

Any more examples or details of fluid-absorptive excipient as disclosed herein would be obvious to a person of ordinary skill in the art. All of them are included in this invention.

### (k) Properties and Composition of Mechanically Strengthening, Semi-Permeable Surface Layer and Strengthening Excipient

The solid core disclosed herein are generally substantially enclosed and supported by a mechanically strengthening surface layer. Generally, said mechanically strengthening surface layer may be somewhat permeable to a relevant physiological fluid under physiological conditions to promote rapid expansion of the solid core upon immersion. That is, the mechanically strengthening surface layer may be fluid-permeable.

In some embodiments, therefore, the diffusivity of a relevant physiological fluid under physiological conditions in at least a mechanically strengthening surface layer is greater than 1×10⁻¹³ m²/s. This includes, but is not limited to a diffusivity of a relevant physiological fluid under physiological conditions in at least one strength-enhancing excipient (or in the strength-enhancing excipient in its totality) greater than 2×10⁻¹³ m²/s, or greater than 5×10⁻¹³ m²/s, or greater than 7×10⁻¹³ m²/s, or greater than 1×10⁻² m²/s, or greater than 2×10⁻¹² m²/s, or greater than 3×10⁻¹² m²/s, or greater than 4×10⁻¹² m²/s, or greater than 5×10⁻¹² m²/s, or greater than 6×10⁻¹² m²/s, or greater than 1×10⁻¹ m²/s. A larger fluid diffusivity is generally preferable for promoting rapid expansion of the solid core.

In some embodiments, moreover, upon immersion of a surface layer-supported solid core (e.g., enclosing surface layer and solid core combined) in a relevant physiological fluid under physiological conditions, mechanically strengthening surface layer reduces or decreases or slows down the rate at which physiological fluid-absorptive excipient is removed, eroded, or dissolved from said solid core. That is, the mechanically strengthening surface layer may be semi-permeable.

In some embodiments, accordingly, upon immersion of a surface layer-supported solid core (e.g., enclosing surface layer and solid core combined) in a relevant physiological fluid under physiological conditions, the diffusivity of at least one physiological fluid-absorptive excipient in or through said mechanically strengthening, semi-permeable surface layer, is no greater than 1×10⁻¹ m²/s. This includes, but is not limited to a diffusivity of at least one physiological fluid-absorptive excipient in or through strength-enhancing excipient, such as a mechanically strengthening, semi-permeable surface layer, no greater than 5×10⁻¹² m²/s, or no greater than 2×10⁻¹² m²/s, or no greater than 1×10⁻¹² m²/s, or no greater than 5×10⁻¹³ m²/s, or no greater than 2×10⁻¹³ m²/s, or no greater than 1×10⁻¹³ m²/s, or no greater than 5×10⁻¹⁴ m²/s, or no greater than 2×10⁻¹⁴ m²/s. Generally, a smaller diffusivity of absorptive excipient through a mechanically strengthening surface layer may be preferable for preserving the integrity of an expanded dosage form.

In some embodiments, furthermore, upon immersion of an element in a relevant physiological fluid under physiological conditions, the diffusivity of at least one physiological fluid-absorptive excipient through mechanically strengthening, semi-permeable surface layer is no greater than 0.3 times the self-diffusivity of said at least one absorptive excipient in a relevant physiological fluid under physiological conditions. This includes, but is not limited to the diffusivity of at least one absorptive excipient through mechanically strengthening, semi-permeable surface layer no greater than 0.2 times, or no greater than 0.1 times, or no greater than 0.05 times, or no greater than 0.02 times, or no greater than 0.01 times, or no greater than 0.005 times, or no greater than 0.002 times, or no greater than 0.001 times the self-diffusivity of said at least one absorptive excipient in a relevant physiological fluid under physiological conditions.

Generally, to assure that a mechanically strengthening, semi-permeable surface layer (e.g., mechanically strengthening excipient) remains a semi-solid or viscoelastic material and stabilizes, or mechanically supports or enforces a core (e.g., one or more elements) after exposure to a physiological fluid (e.g., gastric fluid, etc.), the solubility of said mechanically strengthening, semi-permeable surface layer (e.g., said strengthening excipient) in said physiological fluid may be limited. In some embodiments, therefore, at least one mechanically strengthening second excipient has a solubility no greater than 1 g/l in a relevant physiological/body fluid under physiological conditions. This includes, but is not limited to at least one mechanically strenghtening second excipient (or one or more strengthening excipients, or the strengthening excipient in its totality) having a solubility in a relevant physiological/body fluid under physiological conditions no greater than 1 g/l, or no greater than 0.5 g/l, or no greater than 0.2 g/l, or no greater than 0.1 g/l, or no greater than 0.05 g/l, or no greater than 0.02 g/l, or no greater than 0.01 g/l, or no greater than 0.005 g/l, or no greater than 0.002 g/l, or no greater than 0.001 g/l. In the extreme case, strengthening excipient (e.g., at least one strengthening excipient or the strengthening excipient in its totality) may be insoluble or at least practically insoluble in a relevant physiological fluid under physiological conditions. A smaller solubility of mechanically strengthening, semi-permeable surface layer in physiological fluid is generally preferable for preserving the integrity of an expanded dosage form.

It may be noted that even if the solubility of a relevant physiological fluid is low in a mechanically strengthening, semi-permeable surface layer, said mechanically strengthening, semi-permeable surface layer may soften or plasticize somewhat upon contact with or immersion in said physiological fluid under physiological conditions. As a result, a mechanically strengthening, semi-permeable surface layer (e.g., at least a strength-enhancing excipient) can be a solid in the dry state, but upon immersion in or exposure to a relevant physiological fluid (e.g., gastric fluid, etc.) under physiological conditions, it may transition to a semi-solid or viscoelastic material.

Generally, the mechanical properties (such as stiffness, yield strength, tensile strength, elongational viscosity, etc.) of physiological fluid-soaked mechanically strengthening, semi-permeable surface layer (e.g. physiological fluid-soaked strength-enhancing excipient, physiological fluid-soaked strength-enhancing excipient in its totality, etc.), should be large enough to stabilize or mechanically support the dosage form or drug-containing solid or framework. In the invention herein, the term “physiological fluid-soaked mechanically strengthening, semi-permeable surface layer” is generally referred to as a film mechanically strengthening, semi-permeable surface layer that is/has been immersed in a relevant physiological fluid (e.g., acidic water) for so long that the water concentration in the film is roughly at equilibrium.

However, the stiffness, yield strength, tensile strength, elongational viscosity, etc. of physiological fluid-soaked mechanically strengthening, semi-permeable surface layer should not be too large, so that the expansion of the dosage form or drug-containing solid or framework after exposure to said physiological fluid is not excessively impaired or constrained. Thus, mechanically strengthening, semi-permeable surface layers (e.g., strength-enhancing excipients) that comprise or form a viscoelastic or semi-solid material upon exposure to a relevant physiological fluid are typically preferred herein.

In some embodiments, physiological fluid-soaked mechanically strengthening, semi-permeable surface layer (e.g., physiological fluid-soaked strength-enhancing excipient, physiological fluid-soaked strength-enhancing excipient in its totality, etc.) comprises an elastic modulus, or an elastic-plastic modulus, or a plastic modulus greater than 0.02 MPa. This includes, but is not limited to physiological fluid-soaked mechanically strengthening, semi-permeable surface layer (e.g., physiological fluid-soaked strength-enhancing excipient, physiological fluid-soaked strength-enhancing excipient in its totality, etc.) comprising an elastic modulus, or an elastic-plastic modulus, or a plastic modulus greater than 0.05 MPa, or greater than 0.1 MPa, or greater than 0.2 MPa, or greater than 0.3 MPa, or greater than 0.4 MPa, or greater than 0.5 MPa, or greater than 0.6 MPa, or greater than 0.7 MPa, or greater than 0.8 MPa, or greater than 0.9 MPa, or greater than 1 MPa.

In some embodiments, moreover, physiological fluid-soaked mechanically strengthening, semi-permeable surface layer (e.g., physiological fluid-soaked strength-enhancing excipient, physiological fluid-soaked strength-enhancing excipient in its totality, etc.) comprises an elastic modulus, or an elastic-plastic modulus, or a plastic modulus no greater than about 1000 MPa (e.g., no greater than 500 MPa, or no greater than 200 MPa, or no greater than 100 MPa, or no greater than 50 MPa, or no greater than 20 MPa, or no greater than 10 MPa). Preferably, an elastic modulus of a physiological fluid-soaked strength-enhancing excipient should be greater than about 0.1 MPa and no greater than about 100 MPa.

In some embodiments, moreover, physiological fluid-soaked mechanically strengthening, semi-permeable surface layer (e.g., physiological fluid-soaked strength-enhancing excipient, physiological fluid-soaked strength-enhancing excipient in its totality, etc.) comprises a yield strength greater than 0.005 MPa. This includes, but is not limited to physiological fluid-soaked mechanically strengthening, semi-permeable surface layer (e.g., physiological fluid-soaked strength-enhancing excipient, physiological fluid-soaked strength-enhancing excipient in its totality, etc.) comprising a yield strength greater than 0.0075 MPa, or greater than 0.01 MPa, or greater than 0.02 MPa, or greater than 0.05 MPa, or greater than 0.1 MPa, or greater than 0.2 MPa. In some embodiments, moreover, physiological fluid-soaked mechanically strengthening, semi-permeable surface layer (e.g., physiological fluid-soaked strength-enhancing excipient, physiological fluid-soaked strength-enhancing excipient in its totality, etc.) comprises a yield strength no greater than 500 MPa (e.g., no greater than 200 MPa, or no greater than 100 MPa, or no greater than 75 MPa, or no greater than 50 MPa, or no greater than 20 MPa, or no greater than 10 MPa, or no greater than 5 MPa).

In some embodiments, moreover, physiological fluid-soaked mechanically strengthening, semi-permeable surface layer (e.g., physiological fluid-soaked strength-enhancing excipient, physiological fluid-soaked strength-enhancing excipient in its totality, etc.) comprises a tensile strength greater than 0.02 MPa. This includes, but is not limited to physiological fluid-soaked mechanically strengthening, semi-permeable surface layer (e.g., physiological fluid-soaked strength-enhancing excipient, physiological fluid-soaked strength-enhancing excipient in its totality, etc.) comprising a tensile strength greater than 0.05 MPa, or greater than 0.08 MPa, or greater than 0.1 MPa, or greater than 0.2 MPa, or greater than 0.3 MPa, or greater than 0.4 MPa, or greater than 0.5 MPa, or greater than 0.6 MPa.

In some embodiments, moreover, physiological fluid-soaked mechanically strengthening, semi-permeable surface layer (e.g., physiological fluid-soaked strength-enhancing excipient, physiological fluid-soaked strength-enhancing excipient in its totality, etc.) comprises a tensile strength no greater than 500 MPa (e.g., no greater than 200 MPa, or no greater than 100 MPa, or no greater than 75 MPa, or no greater than 50 MPa, or no greater than 20 MPa, or no greater than 10 MPa).

In some embodiments, moreover, physiological fluid-soaked mechanically strengthening, semi-permeable surface layer (e.g., physiological fluid-soaked strength-enhancing excipient, physiological fluid-soaked strength-enhancing excipient in its totality, etc.) comprises a strain at fracture greater than 0.2. This includes, but is not limited to physiological fluid-soaked mechanically strengthening, semi-permeable surface layer (e.g., physiological fluid-soaked strength-enhancing excipient, physiological fluid-soaked strength-enhancing excipient in its totality, etc.) comprising a strain at fracture greater than 0.5, or greater than 0.75, or greater than 1, or greater than 1.25, or greater than 1.5, or greater than 1.75, or greater than 2, or greater than 2.25, or greater than 2.5. Preferably, the strain at fracture of a physiological fluid-soaked mechanically strengthening semi-permeable surface layer should be greater than about 1.

In some preferred embodiments, physiological fluid-soaked mechanically strengthening, semi-permeable surface layer (e.g., physiological fluid-soaked strength-enhancing excipient, physiological fluid-soaked strength-enhancing excipient in its totality, etc.) is a viscoelastic material. If exposed to a (small) stress for a short time (e.g., for a time smaller than about the relaxation time), it may deform elastically and spring back. If exposed to a (small) stress for a long time (e.g., for a time longer or much longer than about the relaxation time), it may deform plastically.

In some embodiments, upon exposure to a stress for a long time (e.g., for a time longer or much longer than the relaxation time), physiological fluid-soaked mechanically strengthening, semi-permeable surface layer (e.g., physiological fluid-soaked strength-enhancing excipient, physiological fluid-soaked strength-enhancing excipient in its totality, etc.) may deform plastically and essentially behave like a viscous material having an elongational viscosity. In some embodiments, elongational viscosity of physiological fluid-soaked mechanically strengthening, semi-permeable surface layer (e.g., physiological fluid-soaked strength-enhancing excipient, physiological fluid-soaked strength-enhancing excipient in its totality, etc.) is no greater than 1×10¹¹ Pa·s. This includes, but is not limited to elongational viscosity of physiological fluid-soaked mechanically strengthening, semi-permeable surface layer (e.g., physiological fluid-soaked strength-enhancing excipient, physiological fluid-soaked strength-enhancing excipient in its totality, etc.) no greater than 5×10¹⁰ Pa·s, or no greater than 2×10¹⁰ Pa·s, or no greater than 1×10¹⁰ Pa·s, or no greater than 5×10⁹ Pa·s, or no greater than 2×10⁹ Pa·s, or no greater than 1×10⁹ Pa·s.

In some embodiments, moreover, elongational viscosity of physiological fluid-soaked mechanically strengthening, semi-permeable surface layer (e.g., physiological fluid-soaked strength-enhancing excipient, physiological fluid-soaked strength-enhancing excipient in its totality, etc.) is greater than 1×10⁵ Pa·s. This includes, but is not limited to elongational viscosity of physiological fluid-soaked mechanically strengthening, semi-permeable surface layer (e.g., physiological fluid-soaked strength-enhancing excipient, physiological fluid-soaked strength-enhancing excipient in its totality, etc.) greater than 2×10⁵ Pa·s, or greater than 5×10⁵ Pa·s, or greater than 1×10⁶ Pa·s, or greater than 2×10⁶ Pa·s, or greater than 5×10⁶ Pa·s, or greater than 1×10⁷ Pa·s.

In some embodiments, moreover, elongational viscosity of physiological fluid-soaked mechanically strengthening, semi-permeable surface layer (e.g., physiological fluid-soaked strength-enhancing excipient, physiological fluid-soaked strength-enhancing excipient in its totality, etc.) is in the range 1×10⁵ Pa·s-1×10¹¹ Pa·s, and more preferably 5×10⁵ Pa·s-5×10¹⁰ Pa·s, and even more preferably 1×10⁶ Pa·s-2×10¹⁰ Pa·s, and even more preferably 2×10⁶ Pa·s-1×10¹⁰ Pa·s, which includes, but is not limited to elongational viscosity of physiological fluid-soaked mechanically strengthening, semi-permeable surface layer in the range 5×10⁶ Pa·s-5×10⁹ Pa·s.

Furthermore, in some embodiments, the solubility of at least a mechanically strengthening second excipient (or the solubility of mechanically strengthening second excipient in its totality) can differ in different physiological fluids under physiological conditions. By way of example but not by way of limitation, in some embodiments the solubility of at least one mechanically strengthening second excipient in aqueous physiological fluid may depend on the pH value of said physiological fluid. More specifically, in some embodiments at least one mechanically strengthening second excipient can be sparingly-soluble or insoluble or practically insoluble in an aqeuous physiological fluid that is acidic (e.g., in gastric fluid, or in fluid with a pH value smaller than about 4, or in fluid with a pH value smaller than about 5, etc.), but it can be soluble in an aqueous physiological fluid having a greater pH value (e.g., in a fluid with a pH value greater than about 6, or greater than about 6.5, or greater than about 7, or greater than about 7.5, etc.), such as intestinal fluid. A mechanically strengthening second excipient comprising a solubility that is smaller in acidic solutions than in basic solutions is also referred to herein as “enteric excipient”.

In some embodiments, therefore, at least one mechanically strengthening second excipient comprises a solubility in aqueous fluid with a pH value no greater than 4 at least 10 (e.g., at least 20, or at least 50, or at least 100, or at least 200, or at least 500) times smaller than the solubility of said mechanically strengthening second excipient in an aqueous fluid with a pH value greater than 7 (e.g., the latter includes, but is not limited to an aqueous fluid with a pH value greater than 8).

A non-limiting example of such a mechanically strengthening second excipient that is sparingly-soluble in gastric or acidic fluid, but dissolves in intestinal fluid (e.g., aqueous fluid with a pH value greater than about 5.5), is methacrylic acid-ethyl acrylate copolymer.

Another non-limiting example of a mechanically strengthening second excipient is polyvinyl acetate.

Other non-limiting examples of strength-enhancing excipients herein may include methacrylic acid-ethyl acrylate copolymer, methacrylic acic-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose., and so on.

Any more examples or details of strengthening excipient as disclosed herein would be obvious to a person of ordinary skill in the art. All of them are included in this invention.

### (l) Expansion of Drug-Containing Solid and Formation of a Viscoelastic Mass

Generally, a drug-containing solid, a solid core, and so on, may expand with fluid absorption upon ingestion to prevent premature passage through the pylorus and/or to assure that drug is released at the desired rate and/or in the desired time.

In some embodiments of the invention herein, accordingly, at least one dimension (e.g., a side length or the thickness) of a drug-containing solid (e.g., a solid core) expands to at least 1.2 times the initial value (e.g., the initial length prior to exposure to said physiological fluid) within no more than 500 minutes of immersion in a physiological or body fluid under physiological conditions. This includes, but is not limited to at least one dimension of a drug-containing solid (e.g., a solid core) reaching a length at least 1.2 times the initial length within no more than 300 minutes, or within no more than 200 minutes, or within no more than 150 minutes, or within no more than 100 minutes, or within no more than 50 minutes, or within no more than 40 minutes, or within no more than 30 minutes of immersion in said physiological or body fluid under physiological conditions. This may also include, but is not limited to at least one dimension of a drug-containing solid or framework (e.g., a solid core) expanding to a length at least 1.3 times the initial length, or at least 1.4 times the initial length, or at least 1.45 times the initial length, or at least 1.5 times the initial length, or at least 1.55 times the initial length, or at least 1.6 times the initial length within no more than 300 minutes of immersing in or exposing to a physiological or body fluid under physiological conditions.

Furthermore, in some embodiments a drug-containing solid (e.g., a solid core) expands to at least 2 times its initial volume within no more than about 500 minutes of immersing in a physiological or body fluid under physiological conditions. This includes, but is not limited to a drug-containing solid (e.g., a solid core) that expands to at least 2 times, or at least 3 times, or at least 4 times, or at least 4.5 times, or at least 5 times, or at least 6 times, or at least 6.5 times its initial volume within no more than about 300 minutes of immersing in a physiological or body fluid under physiological conditions.

In some embodiments, the drug-containing solid (or the three dimensional structural framework) expands isotropically (e.g., uniformly in all directions) while transitioning to a semi-solid or viscoelastic mass. In the invention herein, a solid mass is generally understood to expand isotropically if the normalized expansion (e.g., the ratio of a length difference and the initial length, such as (L(t)−L₀)/L₀, (H(t)−H₀)/H₀, etc.) deviates by less than about 25-75 percent of its maximum value by changing direction or orientation. Thus, in an isotropically expanding solid, semi-solid mass, or framework, the normalized expansion is roughly the same in all directions. For further information related to isotropic expansion of a drug-containing solid, see, e.g., the International Application No. PCT/US19/19004 filed on Feb. 21, 2019 and titled “Expanding structured dosage form”.

In some embodiments, moreover, upon prolonged exposure to a physiological fluid (e.g., longer than 2, 4, 6, 8, or 10 hours in a lightly stirred dissolution fluid such as acidic water), an expanded framework or semi-solid or viscoelastic mass maintains its length between 1.3 and 4 times the initial length for prolonged time.

In some embodiments, a viscoelastic or semi-solid mass comprises a substantially continuous or connected network of one or more strength-enhancing excipients.

### (m) Mechanical Properties of Expanded Viscoelastic Mass

In some embodiments, moreover a viscoelastic or semi-solid mass (e.g., a viscoelastic composite mass, an expanded drug-containing solid or dosage form, etc.) formed after immersing a drug-containing solid in a physiological fluid under physiological conditions comprises an elastic modulus greater than 0.005 MPa. This includes, but is not limited to a viscoelastic or semi-solid mass (e.g., a viscoelastic composite mass, an expanded drug-containing solid or dosage form, etc.) formed after immersion of a drug-containing solid in a dissolution fluid comprising an elastic modulus greater than 0.007 MPa, or greater than 0.01 MPa, or greater than 0.015 MPa, or greater than 0.02 MPa, or greater than 0.025 MPa, or greater than 0.03 MPa, or greater than 0.035 MPa, or greater than 0.04 MPa, or greater than 0.045 MPa, or greater than 0.05 MPa, or greater than 0.055 MPa, or greater than 0.06 MPa, or greater than 0.065 MPa, or greater than 0.07 MPa, or greater than 0.075 MPa. In some embodiments, therefore, a viscoelastic or semi-solid mass (e.g., a viscoelastic composite mass, an expanded drug-containing solid or dosage form, etc.) formed after immersion of a drug-containing solid in a dissolution fluid is a highly elastic or viscoelastic mass that may not break or permanently deform for prolonged time in a stomach (e.g., under the compressive forces of stomach walls, etc.).

In some embodiments, moreover a viscoelastic or semi-solid mass (e.g., a viscoelastic composite mass, an expanded drug-containing solid or dosage form, etc.) formed after immersing a drug-containing solid in a dissolution fluid comprises an elastic modulus no greater than 50 MPa. This includes, but is not limited to a viscoelastic or semi-solid mass (e.g., a viscoelastic composite mass, an expanded drug-containing solid or dosage form, etc.) comprising an elastic modulus no greater than 40 MPa, or no greater than 30 MPa, or no greater than 20 MPa, or no greater than 10 MPa, or no greater than 5 MPa. The elastic modulus of the viscoelastic or semi-solid may, for example, be limited to prevent injury of the gastrointestinal mucosa.

In some embodiments, moreover a viscoelastic or semi-solid mass formed after immersing a drug-containing solid in a dissolution fluid comprises a yield strength or a fracture strength (or a tensile strength) greater than 0.002 MPa. This includes, but is not limited to a viscoelastic or semi-solid mass formed after immersion of a drug-containing solid in a dissolution fluid comprising a yield strength or a fracture strength greater than 0.005 MPa, or greater than 0.007 MPa, or greater than 0.01 MPa, or greater than 0.02 MPa, or greater than 0.025 MPa, or greater than 0.03 MPa, or greater than 0.035 MPa, or greater than 0.04 MPa, or greater than 0.045 MPa, or greater than 0.05 MPa, or greater than 0.055 MPa, or greater than 0.06 MPa, or greater than 0.065 MPa, or greater than 0.07 MPa, or greater than 0.075 MPa, or greater than 0.8 MPa.

In some embodiments, moreover a viscoelastic or semi-solid mass (e.g., an expanded drug-containing solid or dosage form) formed after immersing a drug-containing solid in a dissolution fluid comprises a yield strength or a fracture strength (or a tensile strength) no greater than 50 MPa. This includes, but is not limited to a viscoelastic or semi-solid mass (e.g., a viscoelastic composite mass, an expanded drug-containing solid or dosage form, etc.) comprising a yield or fracture (or tensile) strength no greater than 20 MPa, or no greater than 10 MPa, or no greater than 5 MPa, or no greater than 2 MPa, or no greater than 1 MPa.

In some embodiments, upon exposure to a physiological fluid a viscoelastic composite mass maintains a tensile strength greater than 0.005 MPa for prolonged time (e.g., for a time longer than 15 hours of exposure to said physiological fluid).

### (n) Drug Release Properties of Dosage Form, Drug-Containing Solid, and Viscoelastic Mass

In some embodiments, moreover, eighty percent of the drug content in a drug-containing solid is released in more than 30 minutes after immersion in a physiological or body fluid under physiological conditions. This includes, but is not limited to a drug-containing solid that releases eighty percent of the drug content in more than than 40 minutes, or in more than 50 minutes, or in more than 60 minutes, or in more than 100 minutes, or in 30 minutes-150 hours, 30 minutes-48 hours, 30 minutes-36 hours, or 45 minutes-24 hours after immersion in a physiological fluid under physiological conditions.

In some embodiments, therefore, upon ingestion of a dosage form, said dosage form is retained in the stomach for a prolonged time to deliver drug into the blood stream over a prolonged time (e.g., 80 percent of the drug is released in 30 mins-200 hours, 1 hour to 200 hours; 1 hour-150 hours; 3 hours-200 hours; 5 hours-200 hours; 3 hours-60 hours; 5 hours-60 hours; 2 hours-30 hours; 5 hours-24 hours; 30 mins-96 hours, 30 mins-72 hours, 30 mins-48 hours, 30 mins-36 hours, 30 mins-24 hours, 1-10 hours, 45 min-10 hours, 30 min-10 hours, 45 min-8 hours, 45 min-6 hours, 30 min-8 hours, 30 min-6 hours, 30 min-5 hours, 30 min-4 hours, etc.) and at a controlled rate. This enables improved control of drug concentration in the blood stream, and improved efficacy or reduced side effects of numerous drug therapies.

### EXPERIMENTAL EXAMPLES

### Part I

In this part, two non-limiting types of dosage form with the same core but different coatings are fabricated and analyzed in vitro and in vivo on dogs. In the first type, the dosage form core is coated with sugar. Because sugar dissolves rapidly in water, such a core may also be considered uncoated. In the second type, the dosage form core is coated with a mechanically strengthening, enteric excipient.

The examples are presented by way of illustration aiming to enable one of skill in the art to more readily understand the invention herein. They are not meant to be limiting in any way.

### Example 1.1: Materials Used for Preparing Dosage Forms

Details of the non-limiting drug, core excipients, gastrointestinal contrast agent (also referred to herein as “contrast agent”), coating excipients, and solvents used for preparing the non-limiting, illustrative dosage forms are as follows.

Drug: Ibuprofen, received as solid particles from BASF, Ludwigshafen, Germany.

Core excipients: (a) Hydroxypropyl methylcellulose (HPMC) with a molecular weight of 120 kg/mol, purchased as solid particles from Sigma, Darmstadt, Germany; (b) Methacrylic acid-ethyl acrylate copolymer (1:1) with a molecular weight of about 250 kg/mol, received as solid particles from Evonik, Essen, Germany (trade name: Eudragit L100-55).

Contrast agent: Barium sulfate (BaSO₄), purchased as solid particles of size ˜1 μm from Humco, Texarkana, Tex.

Excipient of hydrophilic, water-soluble sugar coating: Sucrose (C₁₂H₂₂O₁₁), purchased as solid particles from Sigma, Darmstadt, Germany.

Excipients of mechanically strengthening, enteric coating: (a) Eudragit L100-55 as above; (b) A mixture of 80 wt % polyvinyl acetate and 20 wt % polyvinylpyrrolidone, received as aqueous dispersion from BASF, Ludwigshafen, Germany (trade name: Kollicoat SR).

Solvent used for preparing the core: Dimethylsulfoxide (DMSO) [(CH₃)₂SO], purchased from Alfa Aesar, Ward Hill, Mass.

Solvents used for coating the core: Acetone, ethanol, and deionized water.

### Example 1.2: Preparation of Solid Dosage Form Core

First, particles of ibuprofen (a non-limiting model drug), Eudragit L100-55 (a mechanically strengthening, enteric excipient), and barium sulfate were mixed with liquid DMSO to form a uniform suspension. Then HPMC (a physiological fluid-absorptive excipient) was mixed with the suspension. The respective masses of ibuprofen, Eudragit L100-55, barium sulfate, and HPMC per ml of DMSO in the mixture were 64, 64, 137, and 192 mg/ml DMSO.

The mixture was extruded through a laboratory extruder to form a uniform viscous paste. The viscous paste was put in a syringe equipped with a hypodermic needle of inner radius, Rₙ=84 μm. The paste was extruded through the needle to form a wet fiber that was patterned layer-by-layer as a fibrous dosage form core with cross-ply structure (for further details, see, e.g., the U.S. application Ser. No. 15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, or the International Application No. PCT/US19/52030 filed on Sep. 19, 2019 and titled “Dosage form comprising structured solid-solution framework of sparingly-soluble drug and method for manufacture thereof”). The nominal fiber radius, Rₙ=84 μm, and the nominal inter-fiber spacing, λₙ=450 μm.

After patterning, the solvent was evaporated to solidify the dosage form core. The dosage form core was first put in a vacuum chamber maintained at a pressure of 100 Pa and a temperature of 20° C. for a day. Then it was exposed to an airstream of 60° C. and velocity 1 m/s for 60 min at ambient pressure.

After solvent evaporation, the solid dosage form core consisted of 42 wt % HPMC, 30 wt % barium sulfate, 14 wt % ibuprofen, and 14 wt % Eudragit L100-55. The core was trimmed to a 5 mm thick circular disk with nominal diameter 13-14 mm.

### Example 1.3: Coating the Solid Dosage Form Core

Two types of dosage form were produced. In the first, the core was coated with a hydrophilic sugar coating. The coating solution consisted of ethanol saturated with sucrose; it was held at −20° C. The dosage form was dipped into the coating solution and exposed to a pressure of 200 Pa right after for about an hour to evaporate the ethanol. The dipping-evaporation process was repeated three times.

In the second, the core was coated with a mechanically strengthening, enteric coating. Two coating solutions were used: (a) 1.33 g Eudragit L100-55 in 40 ml acetone, and (b) 2 ml Kollicoat SR dispersion in 20 ml deionized water. Both coating solutions were held at room temperature. The dosage form was dipped into the coating solution and exposed to a pressure of 200 Pa right after for about an hour to evaporate the solvent. The dipping-evaporation process was repeated six times for solution (a) and three times for solution (b).

### Example 1.4 Microstructures of Dosage Forms

The microstructures of the dosage forms with enteric-excipient-coated fibers were imaged by a Zeiss Merlin High Resolution SEM with a GEMINI column. The top surfaces were imaged after coating the sample with a 10-nm thick layer of gold. The cross-sections were imaged after the sample was cut with a thin blade (MX35 Ultra, Thermo Scientific, Waltham, Mass.) and coated with gold as above. The specimens were imaged with either an in-lens secondary electron or a backscattered electron detector, at an accelerating voltage of 5 kV, and a probe current of 95 pA.

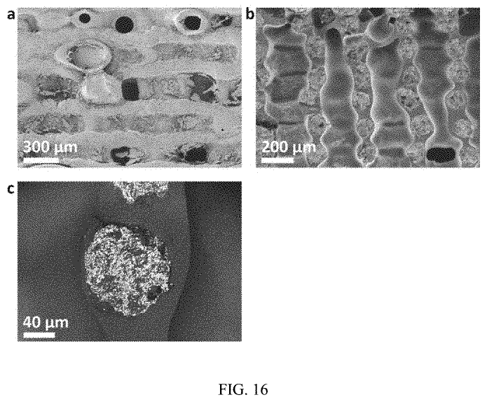

The microstructures of the dosage forms dip-coated with enteric excipient are shown in FIGS. 16a-16c. FIG. 16a illustrates the top view of the dosage forms. The top layer was mostly covered by the coating, but voids of about 100-300 μm in diameter were also present.

FIGS. 16b and 16c show the cross-sectional images. The fibers in the interior were coated; the coating bridged the neighboring fibers vertically, but not horizontally. Thus, the microstructure of the enteric-excipient-coated dosage forms may be approximated as having vertical walls of thickness, 2R₀, and vertical square channels of width, λ₀−2R₀. From FIGS. 16b and 16c, the fiber radius, R₀, was about 65 μm, and the inter-fiber spacing, λ₀, was 280 μm, Table 1.

TABLE 1
Microstructural parameters of the fibrous dosage forms.

| | R₀ (μm) | λ₀ (μm) | φₛ | φ_{f} | φ_{ec} |
|---|---|---|---|---|---|
| Enteric coated | 66 | 280 | 0.72 | 0.59 | 0.13 |

R₀: fiber radius; λ₀: inter-fiber distance; φₛ: volume fraction of solid; φ_{f}: volume fraction of fibers; φ_{ec}: volume fraction of enteric coating.
R₀ and λ₀ were obtained from FIG. 16.
The volume fractions were obtained from Eqs. (22)-(25).
The nominal process parameters were: Rₙ = 84 μm, λₙ = 450 μm.
Moreover, H₀ = 2.5 mm and n₁ = 60.

Several microstructural parameters can be derived for this microstructure. The volume fraction of voids may be expressed as:

$${φ}_{v} = \frac{{( {λ}_{0} - 2 {R}_{0} )}^{2}}{{λ}_{0}^{2}} \qquad ( 22 )$$

The volume fraction of the solid walls (fiber and coating) may be written as:

$${φ}_{s} = 1 - {φ}_{v} = 1 - \frac{{( {λ}_{0} - 2 {R}_{0} )}^{2}}{{λ}_{0}^{2}} = \frac{4 {R}_{0}}{{λ}_{0}} - \frac{4 {R}_{0}^{2}}{{λ}_{0}^{2}} \qquad ( 23 )$$

The volume fraction of fibers (without the coating) may be expressed as:

$${φ}_{f} = ξ \frac{π {R}_{0}}{2 {λ}_{0}} \qquad ( 24 a )$$

where ξ is the ratio of the “nominal” thickness of the dosage form (point contacts between fibers) and the “real” thickness of the dosage form (flattened fiber-to-fiber contacts):

$$ξ = \frac{2 {R}_{0} {n}_{l}}{2 {H}_{0}} = \frac{{R}_{0} {n}_{l}}{{H}_{0}} \qquad ( 24 b )$$

Here n₁ is the number of stacked layers of fibers in the dosage form, and H₀ the half-thickness of the solid dosage form.

Similarly, the volume fraction of the enteric coating may be written as:

$${φ}_{ec} = {φ}_{s} - {φ}_{f} = \frac{4 {R}_{0}}{{λ}_{0}} - \frac{4 {R}_{0}^{2}}{{λ}_{0}^{2}} - ξ \frac{π {R}_{0}}{2 {λ}_{0}} \qquad ( 25 )$$

As listed in Table 1, for the relevant parameters of the dosage forms with enteric-excipient-coated fibers, φₛ=0.72, φ_{f}=0.59, and φ_{ec}=0.13.

The microstructures of the dosage forms dip-coated with sugar were similar to those with the enteric-excipient coating. Because the sugar coating rapidly dissolves upon contact with water or gastric fluid, its volume fraction is not further characterized.

### Example 1.5 Expansion of Dosage Forms

The dosage forms were immersed in a beaker filled with 800 ml dissolution fluid (0.1 M HCl in deionized water at 37° C.). The fluid was stirred with a paddle rotating at 50 rpm. Expansion was monitored by imaging the samples at regular time intervals with a Nikon DX digital camera.

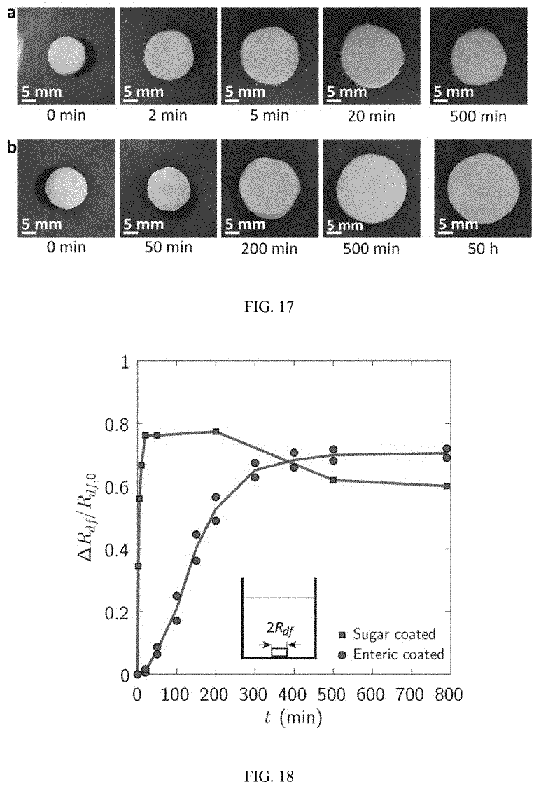

Images of the dosage forms after immersion in the dissolution fluid are shown in FIG. 17. The normalized radial expansion of the dosage forms, ΔR_{df}/R_{df,0}, is plotted versus time in FIG. 18.

As shown in FIG. 17a, the dosage forms with sugar-coated fibers rapidly expanded and transformed into a semi-solid mass. The normalized expansion was 0.56 by 5 min and 0.76 by 20 min. The semi-solid mass was stabilized for over 10 hours, albeit the normalized expansion slightly decreased at longer times, from 0.77 at 200 minutes to 0.6 at 800 minutes, FIGS. 17a and 18.

The dosage forms with enteric-excipient-coated fibers expanded slower; ΔR_{df}/R_{df,0} was about 0.08 at 50 minutes. Then the normalized expansion increased gradually to 0.53 by 200 minutes, and plateaued out to 0.7 by 500 min, FIGS. 17b and 18. Thereafter the dimensions of the expanded dosage forms were unchanged for more than two days.

### Example 1.6 Diametral Compression of Expanded Dosage Forms

All the dosage forms were first soaked in the dissolution fluid (0.1 M HCl in deionized water at 37° C.) until they did not expand any further. The sugar-coated dosage forms were soaked for 30 mins, and the enteric-excipient-coated forms for 6 hours.

Diametral compression tests were then conducted using a Zwick Roell mechanical testing machine equipped with a 10 kN load cell and compression platens. The relative velocity of the platens was 2 mm/s. The test was stopped as soon as the specimen fractured visibly.

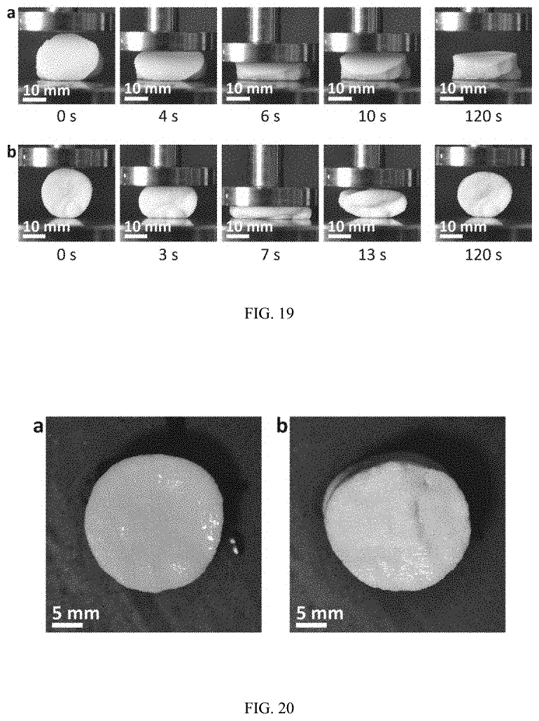

FIG. 19 is a series of images of diametral compression of the expanded dosage forms. The dosage form with sugar-coated fibers could barely support its own weight, FIG. 19a. Upon compression the dosage form deformed further and fractured. As the load was released, the dosage form did not regain its original shape.

The dosage form with enteric-excipient-coated fibers, by contrast, was much stiffer, FIG. 19b. Upon compression, the dosage form deformed, and as the load was released it sprang back and regained a shape and size similar to that of the original form. Nonetheless, the dosage form exhibited a crack along the axis of symmetry after compression, as shown in FIG. 20.

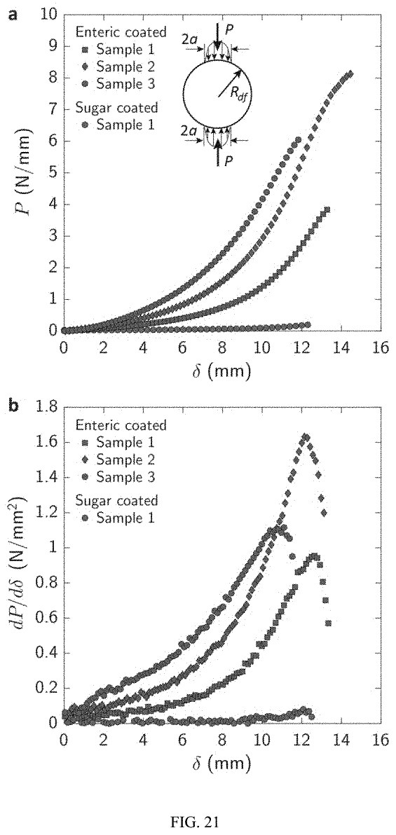

FIG. 21a presents the results of the load per unit length, P, versus displacement, δ, during diametral compression of the two types of dosage form. The slopes, dP/dδ, are plotted in FIG. 21b. For all dosage forms, up to a displacement of about 10-13 mm the load and its slope increased with displacement. But after that the P−δ curve exhibited an inflection point and dP/dδ decreased. At a given displacement, the loads of the enteric-excipient-coated dosage forms were about 20-30 times those of the sugar-coated forms.

### Example 1.7 Elastic Modulus, Load Intensity at Fracture, and Tensile Strength of Expanded Dosage Forms

For data analysis, the expanded dosage form may be considered a linear elastic cylinder of radius, R_{df}, subjected to diametral compression by two hard, flat platens as shown in the inset of FIG. 21a. From the equations of elasticity, for small displacements the relative displacement of the platens may be approximated by (for further details, see, e.g., K. L. Johnson, Contact mechanics, Cambridge University Press, 1985; A. H. Blaesi, N. Saka, Determination of the mechanical properties of solid and cellular polymeric dosage forms, Int. J. Pharm 509, 2016, pp. 444-453):

$$δ = 2 P \frac{1 - {v}^{2}}{π {E}_{df}} ( 2 ln \sqrt{\frac{4 π {R}_{df} {E}_{df}}{P}} - 1 ) \qquad ( 26 )$$

where P is the force per unit length along the cylinder axis, v the Poisson's ratio, and E_{d} the elastic modulus of the expanded dosage form.

By inserting the experimental P and δ values from FIG. 21a in Eq. (26), and using v≈0.5, the elastic modulus of the expanded dosage form can be calculated.

As listed in Table 2, the elastic modulus of the sugar-coated form, E_{df}=0.0075 MPa (7.5 kPa or ˜7.5×10⁻⁶ GPa). The elastic modulus of the expanded enteric-excipient-coated dosage forms, E_{df}=0.184 MPa (˜1.84×10−4 GPa). This modulus is comparable to that of the foams of low-stiffness, highly flexible polymers, such as natural rubber and silicone (for further details about the elastic modulus of a myriad of materials, see, e.g., M. F. Ashby, Materials selection in mechanical design, Third ed., Butterworth-Heinemann, Oxford, 2005).

Excessive plastic deformation, or fracture, of the dosage form is observed if Eq. (26) is severely violated, i.e., if P is at an inflection point or dP/dδ is at a maximum. From FIG. 21, the inflection point, or load at fracture, of the sugar-coated dosage form, P_{f,df}=0.18 N/mm, and that of the enteric-excipient-coated forms, P_{f,df}=4.66 N/mm, Table 2.

TABLE 2
Mechanical properties of expanded fibrous dosage forms.

| | | |
|---|---|---|
| E_{df} (MPa) | P_{f, df} (N/mm) | σ_{f, df} (MPa) |

Sugar coated

| | | | |
|---|---|---|---|
| Sample 1 | 0.0075 | 0.18 | 0.005 |

Enteric coated

| | | | |
|---|---|---|---|
| Sample 1 | 0.111 | 3.31 | 0.096 |
| Sample 2 | 0.201 | 5.61 | 0.162 |
| Sample 3 | 0.240 | 5.05 | 0.146 |
| Average | 0.184 | 4.66 | 0.135 |
| Std | 0.066 | 0.98 | 0.028 |

E_{df}: elastic modulus of expanded dosage form;
P_{f, df}: load per unit length at fracture;
σ_{f, df}: stress at fracture
The properties were obtained from the diametral compression tests reported in FIG. 21, and Eqs. (26) and (27).
The properties of the acidic water-soaked coating film, E = 5.7 MPa and σ_{f} = 1.8 MPa (Table 4 later).

From the load at fracture the tensile strength of the expanded dosage form may be estimated. Under the highly approximate assumption that the displacements are small, the fracture strength is (for further details, see, e.g., K. L. Johnson, Contact mechanics, Cambridge University Press, 1985; A. H. Blaesi, N. Saka, Determination of the mechanical properties of solid and cellular polymeric dosage forms, Int. J. Pharm 509, 2016, pp. 444-453):

$${σ}_{f , df} = \frac{{P}_{f , df}}{π {R}_{df}} \qquad ( 27 )$$

As listed in Table 2, the fracture strength of the sugar-coated dosage form, σ_{f,df}=0.005 MPa, and that of the enteric-excipient-coated form, σ_{f,df}=0.135 MPa. Similar to the elastic modulus, the fracture strength of the enteric-excipient-coated form was more than an order of magnitude greater than that of the sugar-coated form.

Thus, the stiffness and strength of the expanded dosage forms were substantially increased by the enteric coating. However, because even the expanded, enteric-excipient-coated dosage forms are soft materials, they are unlikely to injure the gastric mucosa.

### Example 1.8 Gastric Residence of Dosage Forms in Dogs

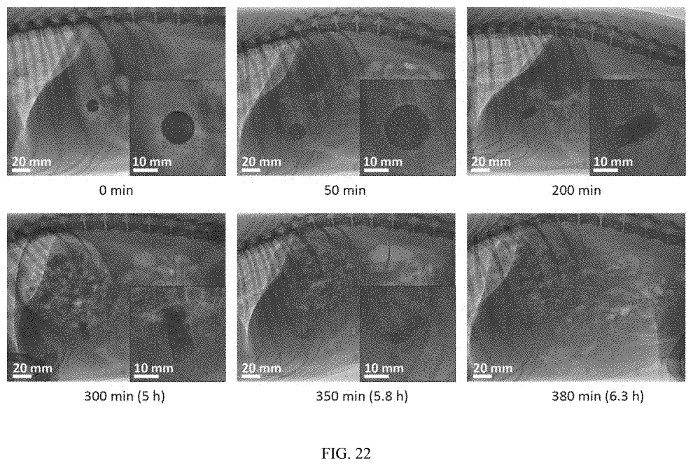

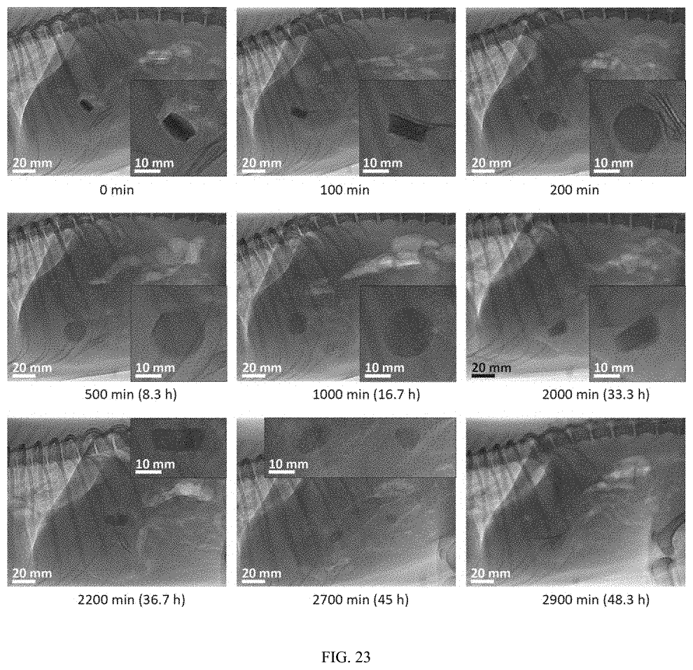

FIGS. 22 and 23 present fluoroscopic images of the dosage forms at various times after administration to a dog.

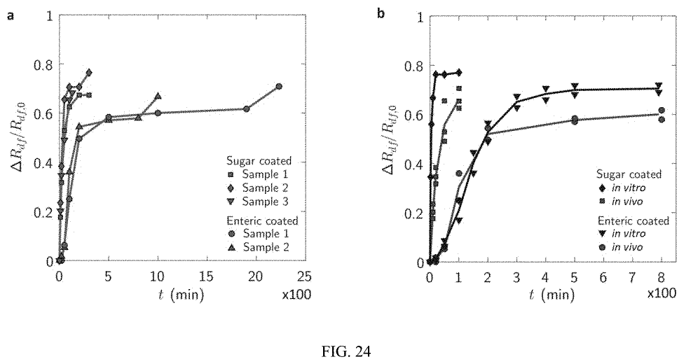

As shown in FIG. 22, the dosage form with sugar-coated fibers passed from the mouth into the stomach in less than a minute. In the stomach it expanded to a normalized radial expansion, ΔR_{df}/R_{fd,0}=0.63 by 100 minutes, and then plateaued to ΔR_{df}/R_{df,0}=0.67, FIG. 24a and Table 3. Thus, the in vivo expansion rate was about a tenth of that measured in vitro, FIG. 24b. After about 300 minutes, as food was given to the dog, the dosage form showed visible cracks. The cracks grew rapidly and resulted in fracture at about 350 minutes. The fragments then passed into the intestines where they dissolved. By about 380 minutes (6.3 hours) the entire dosage form was essentially dissolved.

As shown in FIG. 22, the dosage form with enteric-excipient-coated fibers, too, passed from the mouth into the stomach in less than a minute. Similar to the in vitro results, it then expanded at a moderate rate to a normalized radial expansion ΔR_{df}/R_{df,0}=0.5 by 200 minutes and 0.6 by 500 minutes, FIG. 24b and Table 3. The integrity of the dosage form was mostly preserved until 37-45 hours after ingestion. At 45 hours, fragments were seen in the intestine. The fragments dissolved rapidly; by 48 hours they were essentially invisible, FIG. 22.

Thus, unlike in vitro, in vivo the dosage forms fragmented and dissolved eventually. Fragmentation was due to contraction pulses by the stomach walls that occurred about every 10-30 seconds.

TABLE 3
Properties of fibrous dosage forms in vivo.

| | | | |
|---|---|---|---|
| tₑₓₚ (min) | ΔR_{df}/R_{df, 0} | δₘₐₓ (mm) | tᵣ (h) |

Sugar coated

| | | | | |
|---|---|---|---|---|
| Sample 1 | 100 | 0.67 | 10 | 6.3 |
| Sample 2 | 50 | 0.77 | 10 | 5.5 |
| Sample 3 | 100 | 0.68 | 11 | 2.5 |
| Average | 83 | 0.71 | 10.3 | 4.8 |

Enteric coated

| | | | | |
|---|---|---|---|---|
| Sample 1 | 200 | 0.60 | 6.5 | 41 |
| Sample 2 | 200 | 0.58 | 6.5 | 20 |
| Average | 200 | 0.59 | 6.5 | 30.5 |

tₑₓₚ: time to expand dosage form to greater than 90% of the terminal value;
ΔR_{df}/R_{df, 0}: terminal nominal expansion;
δₘₐₓ: maximum deformation due to contracting stomach walls;
tᵣ: gastric residence time
The data were derived from FIGS. 22-25.

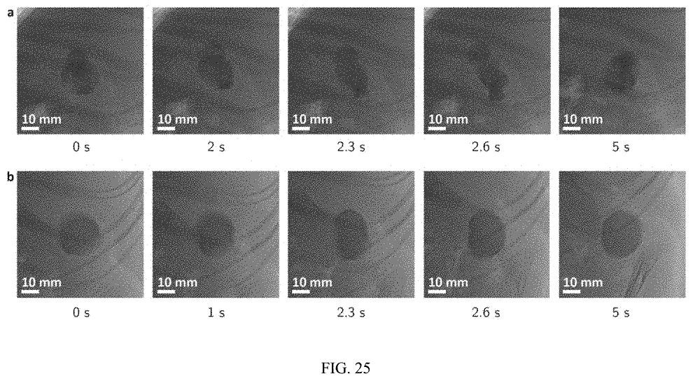

FIG. 25a shows a fluoroscopic image sequence of a dosage form with sugar-coated fibers during a contraction pulse by the stomach walls at about 2 hours after ingestion. The dosage form was circular and of diameter 23 mm initially. At 2.6 s, the dosage form was squeezed by about 11 mm to a width of roughly 12 mm. At 5 s the dosage form regained a round shape of roughly the initial diameter. Soon after the images were taken, however, the dosage form fractured.

FIG. 25b shows a fluoroscopic image sequence of a dosage form with enteric-excipient-coated fibers during a contraction pulse at about 7 hours after ingestion. Initially, the dosage form was circular and of diameter 23 mm. At 1 s, the dosage form was diametrically pinched, and at 2.3 s it was diametrically compressed by about 6.5 mm to a width of about 16.5 mm. The dosage form regained its original shape after about 5 s. The compression-spring back cycles were repeated for several more hours as the dosage form was retained in the stomach.

### Appendix 1A: Solubility and Sorption of Deionized Water with 0.1 M HCl in Eudragit L100-55

Solid films of Eudragit L100-55 were prepared by first dissolving 3 g Eudragit powder in 40 ml acetone. The solution was then poured in a polyethylene box with a flat bottom surface of dimensions 117.6 mm×81.8 mm, and dried at room temperature for about a day. Subsequently, the solid, frozen film was manually detached from the box, and cut into square disks of dimension 30 mm×30 mm using a microtome blade (MX35 Ultra, Thermo Scientific, Waltham, Mass.). The film thickness, 2h₀, was about 250 μm.

For determining the properties of the solid films, they were immersed in the dissolution fluid (water with 0.1 M HCl at 37° C.). The weight of the film, w(t), was measured at different times using a Mettler Toledo analytical balance, and the weight fraction of water, f_{w}(t), in the film determined by:

$${f}_{w} ( t ) = \frac{w ( t ) - {w}_{0}}{w ( t )} \qquad ( 28 )$$

where w₀ is the initial weight of the solid film.

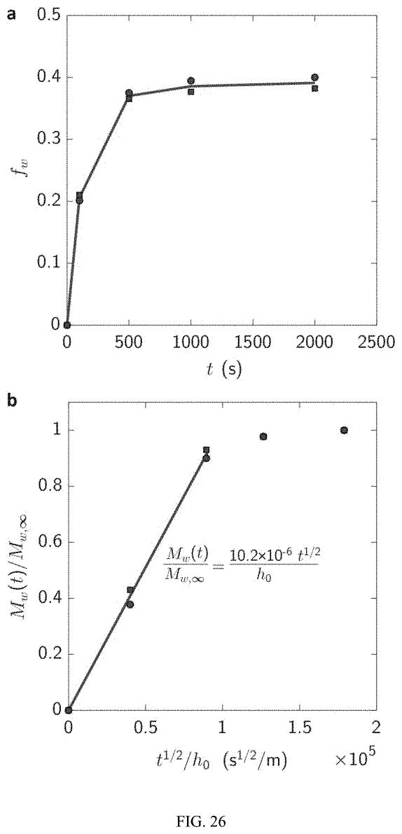

FIG. 26a is a plot of the weight fraction of water in the films versus time after immersion in the dissolution fluid. The weight fraction of water increased with time at a decreasing rate, and plateaued out at about 2000 s to a value of about 0.39. Thus the “solubility” of water in the film was about 390 mg/ml.

FIG. 26b is a plot of the mass of dissolution fluid absorbed by the film at time t, M_{w}(t)=w(t)−w₀, divided by the mass absorbed at “infinite” time (e.g., at 2000 seconds), M_{w,∞}=w(2000)−w₀, versus t^{1/2}/h₀. For small times, the fit of the data was linear; thus the data followed a curve of the form

$$\frac{{M}_{w} ( t )}{{M}_{w , ∞}} = {{k}_{s} ( \frac{t}{{h}_{0}^{2}} )}^{1 / 2} \qquad ( 29 )$$

where the constant, kₛ=5.1×10⁻⁶ m/s^{1/2} (FIG. 26b).

An in-depth analysis of the sorption behaviour of the film is beyond the scope of this paper. However, under the rough assumptions that the diffusivity of water, D_{w}, in the film is constant and the boundary concentration of water is constant and small compared with its density, according to Crank (1975):

$$\frac{{M}_{w} ( t )}{{M}_{w , ∞}} = 1 - {\sum }_{n = 0}^{∞} \frac{8}{{( 2 n + 1 )}^{2} {π}^{2}} exp ( \frac{- {{D}_{w} ( 2 n + 1 )}^{2} {π}^{2} t}{4 {h}_{0}^{2}} ) \qquad ( 30 )$$

For small times, Eq. (30) reduces, roughly, to:

$$\frac{{M}_{w} ( t )}{{M}_{w , ∞}} \approx \frac{2}{\sqrt{π}} {( \frac{{D}_{w} t}{{h}_{0}^{2}} )}^{1 / 2} \qquad ( 31 )$$

Combining Eq. (31) with Eq. (29) the diffusivity of water in the film may be expressed as:

$${D}_{w} = \frac{4 {k}_{s}^{2}}{4} \qquad ( 32 )$$

For the above kₛ value, D_{w}=2×10⁻¹¹ m²/s. This is about the same as the diffusivity in the HPMC-based fiber core (for further details, see, e.g., A. H. Blaesi, N. Saka, Expandable fibrous dosage forms for prolonged drug delivery, Mater. Sci. Eng. C 120, 2021, 110144).

### Appendix 1B: Mechanical Properties of Acidic Water-Soaked Eudragit L100-55 Films

Solid films of Eudragit L100-55 were again prepared by dissolving 3 g Eudragit powder in 40 ml Acetone, pouring the solution in a polyethylene box with dimensions 117.6 mm×81.8 mm, and drying at room temperature for about a day. The solid, frozen films were then punched into tensile specimen according to DIN 53504, type S 3A. The specimen thickness was 150-250 μm.

The tensile specimens were soaked in a dissolution fluid (water with 0.1 M HCl at 37° C.) for about an hour. Subsequently, the water-soaked specimen were loaded in a Zwick Roell Mechanical Testing machine equipped with a 20-N load cell. The initial distance between grips was 28 mm. During testing the grips receded at a relative velocity of 2 mm/s, and the force and distance between grips were recorded. The test was stopped when the sample ruptured, and the load decreased to less than 80% of the maximum load.

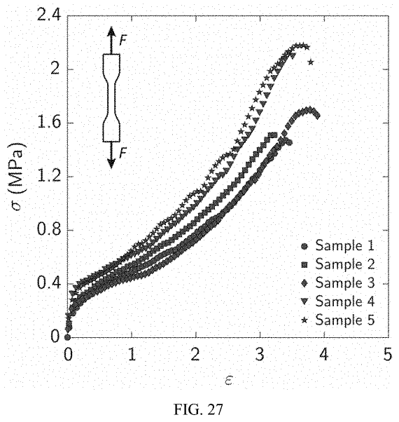

FIG. 27 plots the nominal stress, σ, versus engineering strain, ε, of acidic water-soaked tensile specimen films of the enteric excipient. Initially, the stress increased steeply and roughly linearly with strain. At a strain of about 0.06-0.12, the slope decreased substantially. The stress then increased with strain at a non-linear, progressive rate. Eventually, when the sample ruptured, the stress dropped abruptly.

From the stress-strain curves several properties of the acidic water-soaked films can be derived. The elastic modulus,

$$E = \frac{Δσ}{Δε} {\vert}_{σ << {σ}_{y}} \qquad ( 33 )$$

where the yield strength, σ_{y}, is defined here as the first stress on the curve at which an increase in strain occurs without an increase in stress. The fracture strength, σ_{f}, is the maximum stress on the curve.

As listed in Table 4, the average values of the measured properties, E=5.7 MPa (5.7×10⁻³ GPa), σ_{y}=0.26 MPa, and σ_{f}=1.8 MPa. These values are comparable to the properties of typical low-strength elastomers or rubbers (for further details about the elastic modulus of a myriad of materials, see, e.g., M. F. Ashby, Materials selection in mechanical design, Third ed., Butterworth-Heinemann, Oxford, 2005).

TABLE 4
Properties of acidic water-soaked Eudragit
L100-55 films derived from tension tests.

| | E (MPa) | σ_{y} (MPa) | ε_{y} | σ_{f} (MPa) | ε_{f} |
|---|---|---|---|---|---|
| Sample 1 | 3.6 | 0.19 | 0.10 | 1.48 | 3.43 |
| Sample 2 | 4.9 | 0.26 | 0.12 | 1.52 | 3.21 |
| Sample 3 | 5.7 | 0.21 | 0.06 | 1.70 | 3.76 |
| Sample 4 | 7.3 | 0.34 | 0.08 | 2.14 | 3.47 |
| Sample 5 | 6.9 | 0.30 | 0.09 | 2.18 | 3.64 |
| Average | 5.7 | 0.26 | 0.09 | 1.80 | 3.50 |
| Std | 1.35 | 0.06 | 0.02 | 0.3 | 0.19 |

E: elastic modulus; σ_{y}: yield strength; ε_{y}: strain at yield; σ_{f}: stress at fracture; ε_{f}: strain at fracture.
The properties were obtained from tensile tests reported in FIG. 27.
The elastic modulus, E, was derived from Eq. (33)
σ_{y} was defined as the first stress at which an increase in strain occurred without an increase in stress.
σ_{f} was the maximum stress.

### EXPERIMENTAL EXAMPLES

### Part 2

In this part, three non-limiting fibrous dosage forms as disclosed herein are fabricated and analyzed in vitro and in vivo on pigs. The dosage forms have the same core and coating compositions, but different coating volume fractions, or coating thicknesses. The examples are presented by way of illustration, and aim to enable one of skill in the art to more readily understand the invention. They are not meant to be limiting in any way.

### Example 2.1: Materials Used for Preparing Fibrous Dosage Forms

Details of the non-limiting drug, core excipients, coating excipient, gastrointestinal contrast agent (also referred to herein as “contrast agent”), and solvents used for preparing the non-limiting, illustrative dosage forms are as follows.

Drug: Ibuprofen, received as solid particles from BASF, Ludwigshafen, Germany.

Core excipients: (a) Hydroxypropyl methylcellulose (HPMC) with a molecular weight of 120 kg/mol, purchased as solid particles from Sigma, Darmstadt, Germany; (b) Methacrylic acid-ethyl acrylate copolymer (1:1) with a molecular weight of about 250 kg/mol, received as solid particles from Evonik, Essen, Germany (trade name: Eudragit L100-55).

Coating excipient: Eudragit L100-55 as above.

Contrast agent: Barium sulfate (BaSO₄), purchased as solid particles of size ˜1 μm from Humco, Texarkana, Tex.

Solvent used for preparing the core: Dimethylsulfoxide (DMSO) [(CH₃)₂SO], purchased from Alfa Aesar, Ward Hill, Mass.

Solvent used for coating the core: Acetone.

### Example 2.2: Preparation of Fibrous Dosage Form Core

First, solid particles of ibuprofen, HPMC, Eudragit L100-55, and barium sulfate were mixed with liquid DMSO to form a uniform suspension. The concentrations of ibuprofen, HPMC, Eudragit L100-55, and barium sulfate were 64, 192, 64, and 137 mg/ml DMSO.

Then the suspension was extruded through a laboratory extruder to form a uniform viscous paste. The viscous paste was put in a syringe equipped with a hypodermic needle of inner radius, Rₙ=200 μm. The paste was extruded through the needle to form a wet fiber that was patterned layer-by-layer in a cross-ply structure (for further details, see, e.g., the U.S. application Ser. No. 15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, or the International Application No. PCT/US19/52030 filed on Sep. 19, 2019 and titled “Dosage form comprising structured solid-solution framework of sparingly-soluble drug and method for manufacture thereof”). The nominal fiber radius in the wet, patterned structure, Rₙ, was 200 μm, and the nominal inter-fiber spacing, λₙ, was 820 μm.

After patterning, the solvent was evaporated to solidify the wet, patterned structures. The wet structures were first put in a vacuum chamber maintained at a pressure of 200 Pa and a temperature of 20° C. for a day. Then they were exposed to an airstream of 60° C. and velocity 1 m/s for 60 min at ambient pressure.

After solvent evaporation, the solid structures consisted of 42 wt % HPMC, 30 wt % barium sulfate, 14 wt % ibuprofen, and 14 wt % Eudragit L100-55. They were trimmed to 6 mm thick circular disks, also referred to as “circular fibrous dosage form cores”, “fibrous dosage form cores”, or “fibrous cores”, with nominal diameter 14 mm. The mass of the circular fibrous dosage form cores was about 850 mg, and that of ibuprofen in the cores was about 120 mg.

### Example 2.3: Coating the Fibers of the Fibrous Core

The fibrous dosage form cores produced as above were dip-coated with an enteric coating solution. Three different types of coated dosage form (A, B, and C) were prepared by using three coating solutions of different concentrations. The coating solutions consisted of Eudragit L100-55 and acetone; the concentrations of Eudragit in the solutions were 60 (dosage form A), 100 (B), and 166 mg/ml (C). The coating was applied by dipping the fibrous dosage form cores into the coating solution for about 10-60 seconds. Right after the dip-coated dosage forms were withdrawn from the solution and put in a vacuum chamber to evaporate the solvent. The pressure was slowly reduced to 200 Pa, and maintained at this value for about an hour.

### Example 2.4 Microstructures of Dosage Forms

The microstructures of the both the uncoated and coated fibrous dosage forms were imaged by a Zeiss Merlin High Resolution SEM with a GEMINI column. The top surfaces were imaged after coating the sample with a 10-nm thick layer of gold. The cross-sections were imaged after the sample was cut with a thin blade (MX35 Ultra, Thermo Scientific, Waltham, Mass.) and coated with gold as above. The specimens were imaged with either an in-lens secondary electron, at an accelerating voltage of 5 kV, and a probe current of 95 pA.

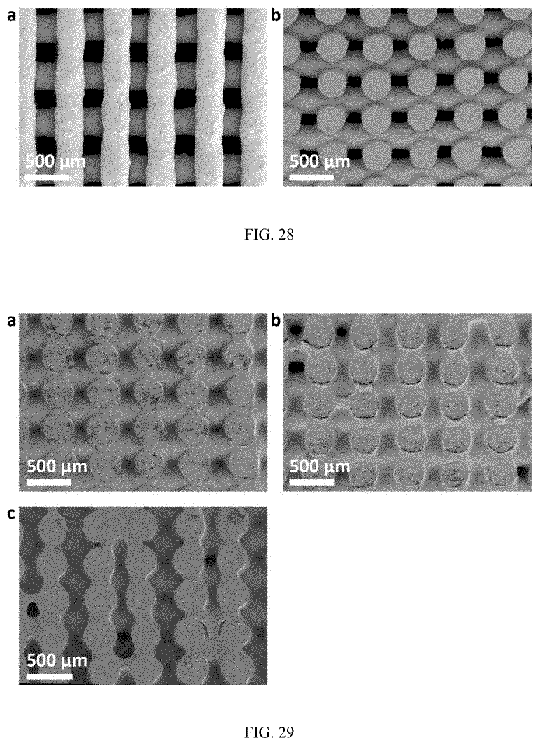

FIGS. 28a and 28b show the top view and the cross section, respectively, of the microstructure of a fibrous dosage form core (e.g., a fibrous dosage form with uncoated fibers). The fiber radius, R₀=305 μm, and the inter-fiber spacing, λ₀=525 μm, Table 5.

As shown in prior work, the volume fraction of fibers (e.g., the volume fraction of solid core) in the dosage form may be expressed as:

$${φ}_{f} = ξ \frac{π {R}_{0}}{2 {λ}_{0}} \qquad ( 34 )$$

where ξ is the ratio of the “nominal” thickness of the dosage form (point contacts between fibers) and the “real” thickness of the dosage form (flattened fiber-to-fiber contacts due to self weight):

$$ξ = \frac{2 {R}_{0} {n}_{l}}{2 {H}_{0}} = \frac{{R}_{0} {n}_{l}}{{H}_{0}} \qquad ( 35 )$$

Here n₁ is the number of stacked layers of fibers in the dosage form, and H₀ the half-thickness of the solid dosage form. For the relevant parameters of the non-limiting experimental dosage forms, φ_{f}=0.67, Table 5.

TABLE 5
Microstructural parameters of the fibrous dosage forms.

| | R₀ (μm) | λ₀ (μm) | c_{c} (mg/ml) | φ_{f} | φᵥ | φ_{c,n} |
|---|---|---|---|---|---|---|
| Uncoated fibers | 153 ± 13 | 528 ± 17 | — | 0.67 | 0.33 | — |
| Coated dosage forms | | | | | | |
| A | 153 ± 13 | 528 ± 17 | 60 | 0.67 | 0.33 | 0.025 |
| B | 153 ± 13 | 528 ± 17 | 100 | 0.67 | 0.33 | 0.041 |
| C | 153 ± 13 | 528 ± 17 | 166 | 0.67 | 0.33 | 0.068 |

R₀: fiber radius
λ₀: inter-fiber distance
c_{coat}: concentration of coating polymer in dip-coating solution
φ_{f}: volume fraction of solid fiber core in dosage form;
φᵥ: volume fraction of voids in uncoated dosage form;
φ_{c,n}: nominal volume fraction of coating in coated dosage form
R₀ and λ₀ were obtained from FIG. 28.
The volume fractions were obtained from Eqs. (34)-(37) using H₀ = 3 mm and nₗ = 29. The nominal fiber radius and inter-fiber spacing respectively were: Rₙ = 400 μm, λₙ = 820 μm.

The volume fraction of voids in the uncoated dosage form core may be written as:

φᵥ=1−φ_{f} (36)

Substituting φ_{f}=0.67 in Eq. (36), φᵥ=0.33.

Micrographs of the cross-sections of the dosage forms dip-coated with the Eudragit polymer-acetone solutions are presented in FIGS. 29a-29c. The coating bridged the neighboring fibers vertically, but generally not horizontally. The amount of coating in the solid dosage forms increased with polymer concentration in the dip-coating solution.

Under the assumption that the mass of Eudragit in the coating of the solid dosage form is the same as the mass of Eudragit in the dip-coating solution that filled the void space, the volume fraction of coating in the solid dosage form may be written as:

$${φ}_{c , n} = \frac{{c}_{c}}{{ρ}_{c}} {φ}_{v} \qquad ( 37 )$$

where c_{c} is the concentration of the coating polymer in the coating solution, ρ_{c} the density of the solid coating polymer. For the relevant parameters of the non-limiting experimental dosage forms, φ_{c,n}=0.025, 0.041, and 0.068, Table 5.

### Example 2.5 Expansion of Dosage Forms

The dosage forms were immersed in a beaker filled with 400 ml dissolution fluid (0.1 M HCl in DI water at 37° C.). The immersed samples were then imaged at regular times by a Nikon DX camera.

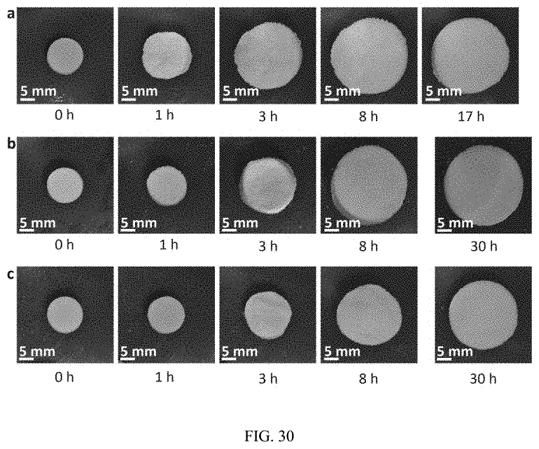

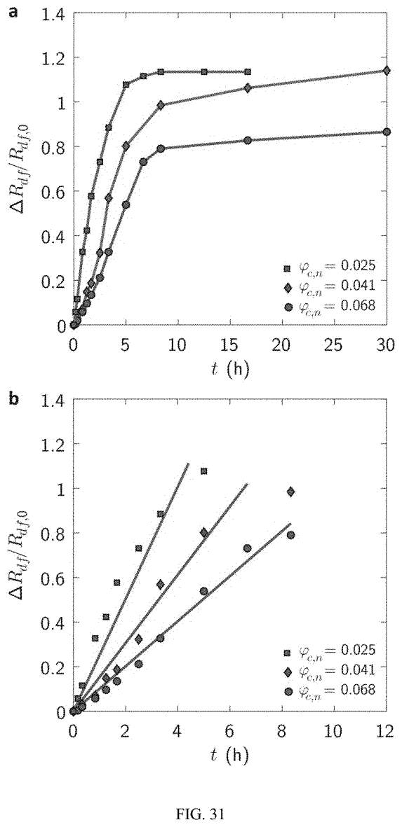

Upon immersion in a dissolution fluid, all the dosage forms expanded and formed a viscoelastic mass, as shown in FIG. 30. The normalized radial expansion of the dosage forms, ΔR_{df}/R_{df,0}, initially increased linearly with time, FIGS. 31a and 31b, and thus could be fitted to an equation of the form ΔR_{df}/R_{df,0}=αt. The normalized expansion rate, α, decreased with volume fraction of the coating, from 0.25/h for dosage form A to 0.1/h for dosage form C, FIG. 31b and Table below. Thus, in agreement with the model equations (2)-(8) and (10), α was roughly proportional to φ_{c,n}/φ_{f}. Eventually, for all dosage forms the expansion stopped, and ΔR_{df}/R_{df,0} remained roughly constant for over a day, FIG. 31a. The times to reach the “terminal expansion”, tₑₓₚ, and the corresponding normalized expansions, ΔR_{df}/R_{df,0|t-texp}, are tabulated below.

| | | α | tₑₓₚ | |
|---|---|---|---|---|
| Dosage form | φ_{c, n} | (1/h) | (h) | ΔR_{df}/R_{df, 0\|t=texp} |
| A | 0.025 | 0.25 | 4.5 | 1.14 |
| B | 0.041 | 0.15 | 6.0 | 0.96 |
| C | 0.068 | 0.1 | 7.5 | 0.78 |

φ_{c, n}: nominal volume fraction of coating in coated dosage form
α: normalized expansion rate
tₑₓₚ time to reach “terminal” expansion
ΔR_{df}/R_{df, 0|t=texp}: “terminal” normalized expansion
The data are obtained from FIGS. 30 and 31.

### Example 2.6 Diametral Compression of Expanded Dosage Forms

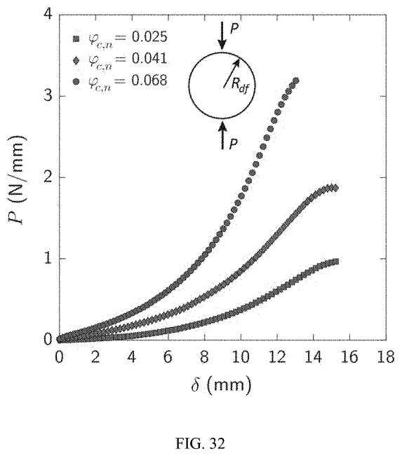

FIG. 32 presents representative results of the load intensity (load per unit length), P, versus displacement, δ, during diametral compression of the expanded dosage forms (at time t=tₑₓₚ). For all dosage forms, up to a displacement of about 10-13 mm the load and ever increased with displacement. But after that the P−δ curve exhibited an inflection point. At a given displacement, P was greater for a greater volume fraction of the coating.

### Example 2.6 Elastic Modulus, Load Intensity at Fracture, and Tensile Strength of Expanded Dosage Forms

A highly approximate estimate of the elastic modulus of the expanded dosage forms, E_{df}, may be obtained by substituting the experimental P and 8 values into the equation:

$$δ = 2 P \frac{1 - {v}^{2}}{π {E}_{df}} ( 2 ln \sqrt{\frac{4 π {R}_{df} {E}_{df}}{P}} - 1 ) \qquad ( 38 )$$

where v the Poisson's ratio of the expanded dosage form.

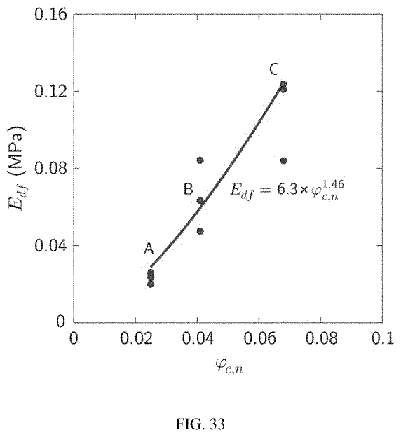

FIG. 33 plots the so-derived elastic modulus of the expanded dosage forms (at time t=tₑₓₚ) versus nominal volume fraction of the coating, φ_{c,n}. The elastic modulus of the expanded dosage forms increased with φ_{c_n}, from 0.023 MPa for dosage form A to 0.11 MPa for dosage form C. The results could be fitted to the curve E_{df}=6.3×φ_{c,n}^{1.46}.

An estimate of the load intensity at the onset of fracture of the expanded dosage forms may be considered when P is at the inflection point. FIG. 34a plots the load intensity at fracture, P_{f,df}, obtained from the experimental P−δ curves at t=tₑₓₚ versus nominal volume fraction of the coating, φ_{c,n}. P_{f,df} increased with φ_{c,n}, from 0.81 N/mm for dosage form A to 2.56 N/mm for dosage form C, Table 6. The results could be fitted to P_{f,df}=63×φ_{c,n}^{1.19}, FIG. 34a.

From the load at fracture the tensile strength of the expanded dosage form may be estimated as:

$${σ}_{f , df} = \frac{{P}_{f , df}}{π {R}_{df}} \qquad ( 39 )$$

As listed in Table 6, using the load intensities calculated above and R_{df}=12 mm, σ_{f,df} increased from 0.022 MPa for dosage form A (φ_{c,n}=0.025) to 0.068 MPa for dosage form C (φ_{c,n}=0.068). The results could be fitted to the curve of σ_{f,df}=1.67×φ_{c,n}^{1.19}, FIG. 34b.

### Example 2.7 Static Fatigue Strength of Expanded Dosage Forms

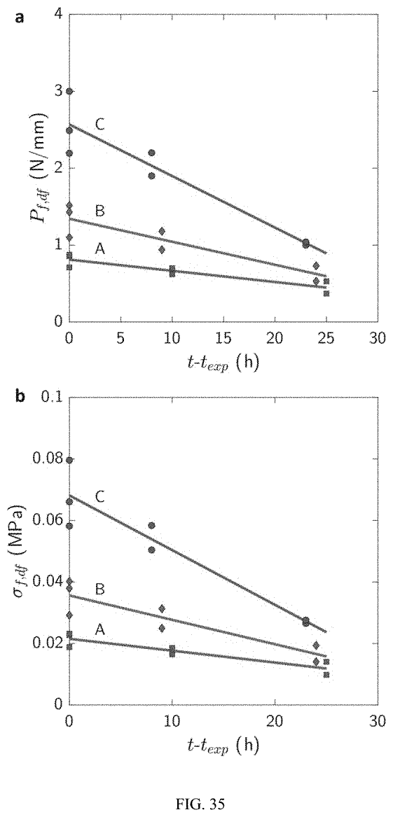

FIG. 35 plots the load intensity at fracture, P_{f,df}, and the corresponding tensile strength, σ_{f,df}, versus time after expansion, t−tₑₓₚ. Both P_{f,df} and σ_{f,df} decreased linearly with time. The rate of decrease increased with volume fraction of the coating (i.e., with “initial” strength), FIG. 35 and Table 6. For all dosage forms, P_{f,df}, and σ_{f,df} decreased to about half the “initial” value in 20-30 hours after expansion. Moreover, as shown in FIG. 36, the results could be fitted to an equation of the form of the model Eq. (14).

### Example 2.8 Gastric Residence of the Dosage Forms in Pigs

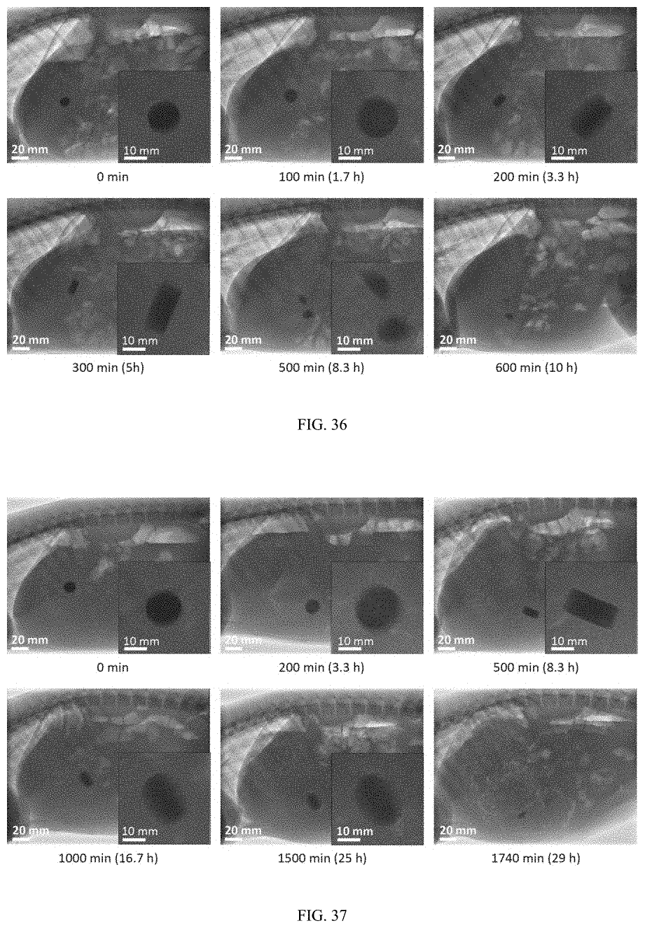

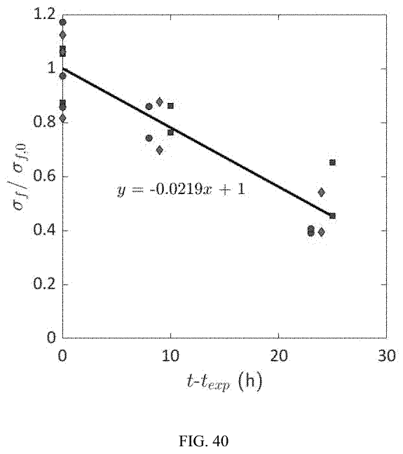

The position, size and shape of dosage forms A-C after administration to pigs is presented in FIGS. 37-39. The normalized expansion of the dosage forms is plotted versus time in FIG. 40.

FIG. 37 shows dosage form A at various times after administration. Upon entering the stomach the dosage form expanded linearly with time to a normalized radial expansion, ΔR_{df}/R_{df,0}=0.57, by 300 minutes, FIG. 40. At 500 minutes, the dosage form fragmented in the stomach. The fragments disintegrated and dissolved rapidly; by 600 minutes they were barely visible.

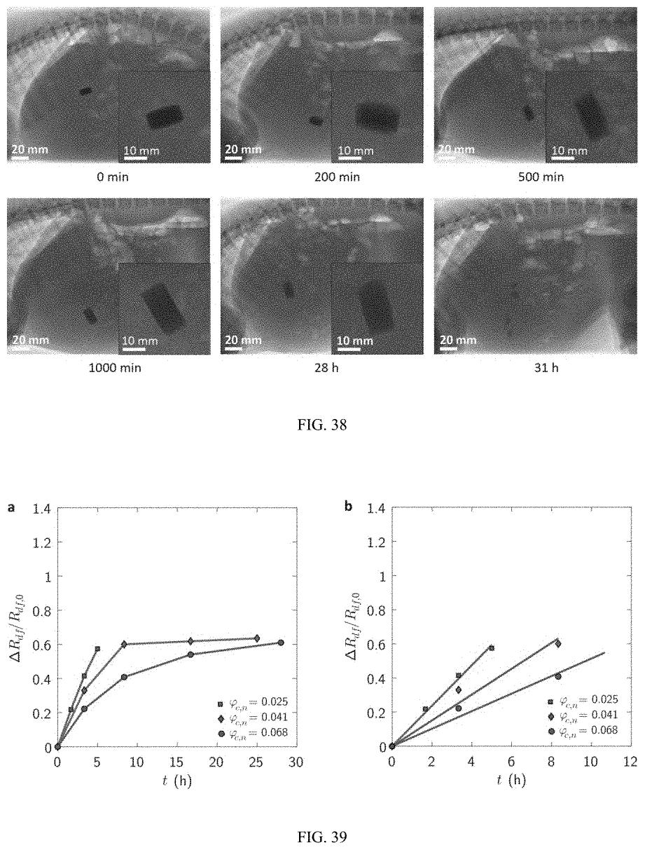

FIGS. 38 and 39 present fluoroscopic images of dosage forms B and C at various times after administration. Upon entering the stomach the dosage forms expanded to a normalized expansion, ΔR_{df}/R_{df,0}, greater than 0.5 in about 400 (dosage form B) and 600 minutes (dosage form C), respectively, FIG. 40. Eventually, after 27 and 31 hours in the stomach the dosage forms fragmented and dissolved.

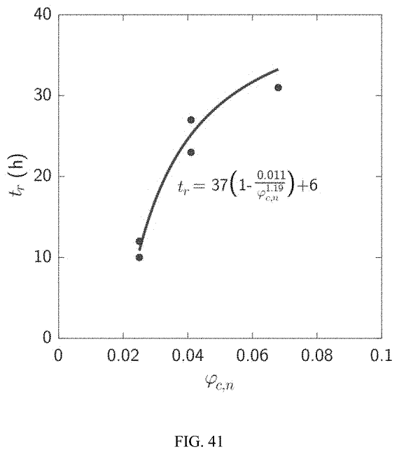

Table 6 summarizes the expansion and gastric residence time of the dosage forms in vivo, and compares them with the properties in vitro. Moreover, as shown in FIG. 41, the gastric residence time of the dosage forms could be fitted to an equation of the form of the model Eq. (16).

TABLE 6
In vitro and in vivo properties of gastroretentive fibrous dosage forms.

| | in vitro properties | | | | | | | | in vivo properties | | | |
|---|---|---|---|---|---|---|---|---|---|---|---|---|
| | | | ΔR_{df}/ | | | | | | | | ΔR_{df}/ | |
| | α | tₑₓₚ | R_{df,0}\| | E_{df} | P_{f,df}\|ₜ₌ₜₑₓₚ | σ_{f,df}\|ₜ₌ₜₑₓₚ | dP_{f,df}/dt | dσ_{f,df}/dt | α | tₑₓₚ | R_{df,0}\| | tᵣ |
| | (1/h) | (h) | ₜ₌ₜₑₓₚ | (MPa) | (N/mm) | (MPa) | (N/mmh) | (MPa/h) | (1/h) | (h) | ₜ₌ₜₑₓₚ | (h) |
| Dosage | | | | | | | | | | | | |
| form | | | | | | | | | | | | |
| A | 0.25 | 4.5 | 1.14 | 0.023 | 0.81 | 0.022 | −1.46 × 10⁻² | −3.86 × 10⁻⁴ | 0.119 | 4.8 | 0.57 | 11^{a} |
| B | 0.15 | 6.0 | 0.96 | 0.065 | 1.35 | 0.036 | −2.99 × 10⁻² | −7.93 × 10⁻⁴ | 0.076 | 7.9 | 0.60 | 25^{a} |
| C | 0.10 | 7.5 | 0.78 | 0.110 | 2.56 | 0.068 | −6.48 × 10⁻² | −1.72 × 10⁻³ | 0.052 | 10.5 | 0.54 | 31 |

α: normalized expansion rate;
tₑₓₚ: time to reach “terminal” expansion;
ΔR/R₀|ₜ₌ₜₑₓₚ: “terminal” normalized expansion;
E_{df}: elastic modulus of expanded dosage form at time t = tₑₓₚ;
P_{f,df}|ₜ₌ₜₑₓₚ: load intensity at fracture of expanded dosage form at t = tₑₓₚ;
σ_{f,df}|ₜ₌ₜₑₓₚ: fracture strength of dosage form at t = tₑₓₚ;
dP_{f,df}/dt: rate of decrease of load intensity at fracture of expanded dosage form
dσ_{f,df}/dt: rate of decrease of fracture strength of expanded dosage form;
tᵣ: gastric residence time.
α, tₑₓₚ, and ΔR_{df}/R_{df,0}|ₜ₌ₜₑₓₚ were obtained from FIG. 31 (in vitro) and FIG. 39 (in vivo). E_{df} was obtained from FIG. 33, P_{f,df}|ₜ₌ₜₑₓₚ and σ_{f,df}|ₜ₌ₜₑₓₚ were obtained from FIG. 34, and dP_{f,df}/dt and dσ_{f,df}/dt were obtained from FIG. 35. tᵣ was obtained from FIGS. 36-38.
^{a}Average of two samples. The gastric residence times of the individual samples were 10 h and 12 h (dosage form A), and 27 h and 23 h (dosage form B).

### Appendix 2A Viscosity of Acidic Water-Soaked Eudragit L100-55 Films (e.g., a Mechanically Strengthening, Semi-Permeable Surface Layer)

Solid films of Eudragit L100-55 were prepared by dissolving 3 g Eudragit powder in 40 ml Acetone, pouring the solution in a polyethylene box with dimensions 117.6 mm×81.8 mm, and drying at room temperature for about a day. The solid, frozen films were then punched into tensile specimen according to DIN 53504, type S 3A. The specimen thickness was 150-250 μm.

The tensile specimens were soaked in a dissolution fluid (water with 0.1 M HCl at 37° C.) for about an hour. Subsequently, one end of the specimen was attached to a sample holder that was held in the dissolution fluid. A weight of either 0.2, 0.5, 1, or 2 g was attached to the other end of the specimen to induce a stress of 1.9, 6.9, 13.8, and 42.9 kPa. The length of the stressed specimen then was monitored over time.

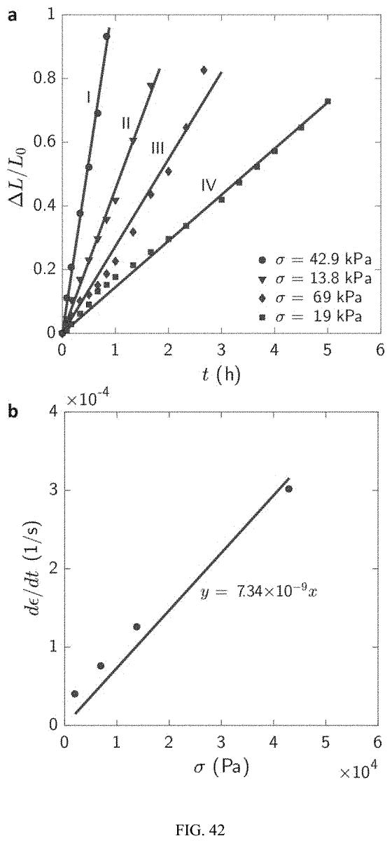

FIG. 42a presents the engineering strain, ΔL/L₀, of the specimens versus time. Up to a strain of about 1, ΔL/L₀ increased linearly with time (i.e., at constant strain rate).

FIG. 42b plots the strain rate, dε/dt, versus the applied stress, σ. The strain rate increased roughly linearly with stress as dε/dt=7.34×10⁻⁹σ.

Thus, the strain rate can be approximated by an adapted form of Hooke's law as:

$$\frac{d ε}{dt} = \frac{σ}{η} \qquad ( 40 )$$

where η is the elongational viscosity of the specimen. From FIG. 42b, the elongational viscosity of the non-limiting experimental specimen, η=1.36×10⁸ Pa·s.

### APPLICATION EXAMPLES

In some embodiments, the amount of active ingredient contained in a dosage form disclosed in this invention is appropriate for administration in a therapeutic regimen that shows a statistically significant probability of achieving a predetermined therapeutic effect when administered to a relevant population. By way of example but not by way of limitation, active ingredients may be selected from the group consisting of acetaminophen, aspirin, caffeine, ibuprofen, an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antihyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, or a vasodilator.

Moreover, while useful for improving almost any drug therapy, the disclosed dosage forms can be particularly beneficial for therapies that require tight control of the concentration in blood of drugs that are soluble or fairly soluble in acidic but sparingly soluble or practically insoluble in basic solution.

In some embodiments, therefore, the dosage form herein comprises at least one active pharmaceutical ingredient having a solubility that is at least five times greater in acidic solution than in basic solution. This includes, but is not limited to at least one active ingredient having a solubility that is at least 10 times, or at least 15 times, or at least 20 times, or at least 30 times, or at least 50 times greater in acidic solution than in basic solution. In the invention herein, a solution is understood “acidic” if the pH value of said solution is no greater than about 5.5. A solution is understood “basic” if the pH value of said solution is greater than about 5.5.

Moreover, in some embodiments, the dosage form herein comprises at least one active pharmaceutical ingredient that is a basic compound. In the invention herein, a compound is understood “basic” if the acid dissociation constant (e.g., the pKa value) of said compound is greater than about 5.5.

More generally, furthermore, the disclosed dosage forms can be beneficial for therapies that require tight or fairly tight control of the concentration in blood of drugs that are sparingly-soluble (e.g., poorly soluble) in an aqueous physiological fluid or gastro-intestinal fluid.

Thus, in some embodiments, the dosage form herein comprises at least one active pharmaceutical ingredient having a solubility no greater than 5 g/l in an aqueous physiological/body fluid under physiological conditions. This includes, but is not limited to at least one active ingredient having a solubility no greater than 2 g/l, or no greater than 1 g/l, or no greater than 0.5 g/l, or no greater than 0.2 g/l, or no greater than 0.1 g/l in an aqueous physiological or body fluid under physiological conditions.

It may be noted, moreover, that due to the greater bioavailability, with the disclosed dosage form the mass of drug a patient is recommended to or supposed to ingest to achieve a therapeutic effect may be lower than with the traditional dosage form.

Additionally, due to the capability of releasing drug into the upper gastrointestinal tract over prolonged time, the disclosed dosage form may enable to reduce the dosing frequency for treatment of a specific disease or medical condition.

The disclosed dosage form, therefore, can be beneficial for therapies comprising a drug with short half-life in blood or a human or animal body. The “half-life” is understood herein as the period of time required for a “maximum” concentration or “maximum” amount of drug in blood or in the body to be reduced by one-half, under the condition that no drug is delivered into the blood or body during said time period. The concentration of drug in blood may generally be estimated from measurements of the concentration of drug in blood plasma.

In some embodiments, accordingly, the dosage form herein comprises at least one active pharmaceutical ingredient having a half-life in a human or animal body (e.g., a physiological system) no greater than one day or 24 hours. This includes, but is not limited to a half-life in a human or animal body no greater than 22 hours, or no greater than 20 hours, or no greater than 18 hours, or no greater than 16 hours, or no greater than 14 hours, or no greater than 12 hours, or no greater than 10 hours, or no greater than 8 hours, or no greater than 6 hours, or no greater than 4 hours, or in the ranges 0.5-24 hours, 0.5-20 hours, 0.5-16 hours, 0.5-12 hours, 0.5-10 hours, 0.5-8 hours, or 0.5-6 hours.

Additionally, the disclosed dosage forms can be manufactured by an economical process enabling more personalized medicine.

It would be obvious to a person of ordinary skill in the art that the above-listed application examples are just a list of non-limiting examples, and that many more applications can be found for the dosage forms disclosed herein. All such applications not mentioned here but obvious to a person of ordinary skill in the art are included in this invention.

We claim:

1. A pharmaceutical dosage form comprising:
a drug-containing solid having a fluid-absorptive solid core and a mechanically strengthening, semi-permeable surface layer;
said fluid-absorptive solid core comprising at least a fluid-absorptive first excipient;
said fluid-absorptive solid core further substantially encapsulated by said mechanically strengthening, semi-permeable surface layer, said semi-permeable surface layer comprising at least a mechanically strengthening second excipient;
whereby
upon exposure of the dosage form to a physiological fluid, the surface layer-encapsulated solid core expands primarily with fluid absorption, thereby transitioning to a viscous or semi-solid mass; and
the mechanically strengthening, semi-permeable surface layer forms a semi-permeable, viscoelastic membrane; and
the drug-containing solid forms an expanded, viscoelastic composite mass.

2. The dosage form of claim 1, wherein the fluid-absorptive solid core has at least one dimension greater than 6 mm.

3. The dosage form of claim 1, wherein the solubility of a physiological fluid in one or more fluid-absorptive excipients is greater than 600 mg/ml.

4. The dosage form of claim 1, wherein upon exposure to a physiological fluid under physiological conditions, the diffusivity of said physiological fluid through a fluid-absorptive core is greater than 0.2×10⁻² m²/s (e.g., greater than 0.5×10⁻² m²/s, or greater than 10-12 m²/s).

5. The dosage form of claim 1, wherein at least one fluid-absorptive excipient comprises hydroxypropyl methylcellulose.

6. The dosage form of claim 1, wherein at least one fluid-absorptive excipient is selected from the group comprising hydroxypropyl methylcellulose, hydroxyethyl cellulose, polyvinyl alcohol, polyvinylpyrrolidone, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), polyacrylic acid, polyethylene oxide, or
vinylpyrrolidone-vinyl acetate copolymer.

7. The dosage form of claim 1, wherein volume or weight fraction of one or more fluid-absorptive excipients in the fluid-absorptive solid core is greater than 0.1.

8. The dosage form of claim 1, wherein the solubility of a mechanically strengthening second excipient is no greater than 0.1 mg/ml in a relevant physiological fluid (e.g., gastric fluid) under physiological conditions.

9. The dosage form of claim 1, wherein the solubility of a relevant physiological fluid in at least one mechanically strengthening second excipient is no greater than 750 mg/ml under physiological conditions.

10. The dosage form of claim 1, wherein at least a mechanically strengthening second excipient (or the strength-enhancing excipient in its totality, or a mechanically strengthening, semi-permeable surface layer) comprises a strain at fracture greater than 0.5 after soaking with a physiological fluid under physiological conditions.

11. The dosage form of claim 1, wherein at least one mechanically strengthening second excipient (or the strength-enhancing excipient in its totality, or a mechanically strengthening, semi-permeable surface layer) comprises a tensile strength in the range of 0.05 MPa-100 MPa after soaking with a physiological fluid under physiological conditions.

12. The dosage form of claim 1, wherein elongational viscosity of mechanically strengthening, semi-permeable surface layer is in the range of 5×10⁵ Pa·s-1×10¹¹ Pa·s after soaking with a physiological fluid under physiological conditions.

13. The dosage form of claim 1, wherein at least one mechanically strengthening second excipient comprises an enteric polymer.

14. The dosage form of claim 1, wherein at least one mechanically strengthening second excipient comprises methacrylic acid-ethyl acrylate copolymer.

15. The dosage form of claim 1, wherein at least one mechanically strengthening second excipient comprises polyvinyl acetate.

16. The dosage form of claim 1, wherein at least one mechanically strengthening second excipient is selected from the group comprising methacrylic acid-ethyl acrylate copolymer, methacrylic acic-methyl methacrylate copolymer, ethyl acrylate-methylmethacrylate copolymer, hydroxypropyl methylcellulose acetate succinate, polyvinyl acetate, polymers including methacrylic acid, polymers including ethyl acrylate, polymers including methyl methacrylate, polymers including methacrylate, Poly[Ethyl acrylate, methyl methacrylate, trimethylammonioethyl methacrylate chloride], and ethylcellulose.

17. The dosage form of claim 1, wherein said fluid-absorptive solid core comprises at least a drug.

18. The dosage form of claim 1, wherein upon exposure to a physiological fluid, the drug-containing solid or semi-solid releases drug over time.

19. The dosage form of claim 1, wherein said fluid-absorptive solid core comprises a three-dimensional structural framework of structural elements.

20. The dosage form of claim 19, wherein average thickness of one or more structural elements is in the range between 10 μm and 2.5 mm.

21. The dosage form of claim 19, wherein one or more structural elements are repeatably arranged.

22. The dosage form of claim 19, wherein one or more elements comprise segments spaced apart from adjoining segments by element-free spacings, thereby defining one or more element-free spaces in the drug-containing solid.

23. The dosage form of claim 22, wherein at least one free space is filled with matter removable by a physiological fluid under physiological conditions.

24. The dosage form of claim 22, wherein at least one free space is filled with a matter comprising a gas.

25. The dosage form of claim 19, wherein one or more structural elements comprise one or more fibers.

26. The dosage form of claim 1, wherein said fluid-absorptive solid core comprises a three-dimensional structural network of criss-crossed stacked layers of fibers.

27. A pharmaceutical dosage form comprising:
a drug-containing solid having a fluid-absorptive solid core and a semi-permeable surface layer;
said fluid-absorptive solid core comprising a three-dimensional structural framework of one or more structural elements;
said elements comprising at least a first excipient, wherein said first excipient includes at least a fluid-absorptive polymeric constituent;
said semi-permeable surface layer substantially encapsulating said elements;
said semi-permeable surface layer further comprising at least a second excipient, said second excipient including at least a mechanically strengthening polymeric constituent;
whereby
upon exposure of the dosage form to physiological fluid, the surface layer-supported solid core expands with fluid absorption, thereby forming a viscoelastic composite mass.

28. A pharmaceutical dosage form comprising:
a drug-containing solid having a fluid-absorptive solid core and a semi-permeable surface layer;
said fluid-absorptive solid core comprising a three-dimensional structural network of criss-crossed stacked layers of fibers;
said fibers comprising at least a first excipient, wherein said first excipient includes at least a fluid-absorptive polymeric constituent;
said semi-permeable surface layer substantially encapsulating said fibers;
said semi-permeable surface layer further comprising at least a second excipient, said second excipient including at least a mechanically strengthening polymeric constituent;
whereby
upon exposure of the dosage form to physiological fluid, the surface layer-supported solid core expands with fluid absorption, thereby forming a viscoelastic composite mass.
